(19) United States
(12) Patent Application Publication
(10) Pub. No.: US 2024/0117115 A1
(43) Pub. Date: Apr. 11, 2024

(54) POLY(CYCLIC ACETAL)S, METHODS OF MAKING SAME, AND USES THEREOF

(71) Applicant: Cornell University, Ithaca, NY (US)

(72) Inventors: Geoffrey W. Coates, Lansing, NY (US); Brooks Abel, Ithaca, NY (US); Rachel Snyder, Ithaca, NY (US); Holley Grace Hester, Ithaca, NY (US)

(21) Appl. No.: 18/262,509

(22) PCT Filed: Jan. 21, 2022
(86) PCT No.: PCT/US22/13424

§ 371 Date: Jul. 21, 2023

Related U.S. Application Data
(60) Provisional application No. 63/140,076, filed on Jan. 21, 2021.

(51) Int. Cl. C08G 65/16; C07D 317/12; C08G 65/26

(52) U.S. Cl. CPC C08G 65/16; C07D 317/12; C08G 65/266; C08G 2650/22; C08G 2650/44

(57) ABSTRACT

Poly(cyclic acetal)s, methods of making same, and uses of same. The poly(cyclic acetal)s may have a number average molecular weight (Mn) of 10 to 3000 kiloDaltons (kDa) and over 50% of the chain ends may exclude hydroxyl groups. The poly(cyclic acetal) may be a homopolymer or copolymer(s) of poly(1,3-dioxolane) (PDXL). The poly(cyclic acetal)s may have one or more or all of: a thermal stability (Td,5%) of 337° C. to 392° C.; a thermal stability of (Td.50%) of 377° C. to 462° C.; or an Arrhenius activation energy (Ea) of 85.0 kJ/mol with 2 mol % of strong acid (e.g., pKa less than or equal to 4). Methods of polymerizing poly(cyclic acetal)s may comprise reacting cyclic acetal monomers with either Lewis acid catalysts and haloalkyl ether initiators or organic cation salt catalyst(s) and proton traps. Methods of chemically recycling poly(cyclic acetal)s into cyclic acetals may react poly(cyclic acetal)s with strong acids.

### CROSS-REFERENCE TO RELATED APPLICATIONS

This application claims the benefit of U.S. Provisional Patent Application No. 63/140,076, filed Jan. 21, 2021, the contents of the above-identified application are hereby fully incorporated herein by reference in their entirety.

### STATEMENT REGARDING FEDERALLY SPONSORED RESEARCH

This invention was made with government support awarded by the Joint Center for Energy Storage Research (JCESR), an Energy Innovation Hub funded by the U.S. Department of Energy, Office of Science, Office of Basic Energy Science, under contract no. DE-AC02-06CH11357 and awarded by the National Science Foundation under contract nos. 1719875 and 1531632. The government has certain rights in the invention.

### BACKGROUND OF THE DISCLOSURE

Plastics represent the most high-performance, versatile, and cost-effective materials available today and are essential for applications including healthcare supplies, packaging, and technology. Unfortunately, the mass manufacture and uncontrolled disposal of plastics has come at both economic and environmental costs. Currently, most collected waste materials are processed via downcycling approaches such as mechanical recycling—a process that affords lower value materials with diminished mechanical properties. Chemical recycling and upcycling are more promising strategies to combat the plastic problem, enabling plastic waste to be used as feedstocks for value-added materials via either pyrolysis for energy recovery; conversion to superfine chemicals; or chemical recycling to monomer (CRM). In particular, CRM allows plastic waste to be converted directly back to monomer feedstocks, enabling a circular economy that both mitigates the need for continuous petrochemical sourcing and could potentially eliminate the accumulation of plastic waste. In order to reduce our dependence on fossil fuel, prevent the accumulation of billions of tons of plastic each year, and mitigate massive economic losses, the development of a circular polymer economy via CRM must be at the forefront of sustainability efforts.

CRM was first envisioned by Höcker, who proposed that moderately—strained heterocyclic monomers with ceiling temperatures between −78 to 250° C. could be used to reform plastic sustainability. To date, the polymerization and subsequent depolymerization of polymers has been demonstrated in polyesters, polycarbonates, heterocyclic monomers, and polyolefins. Chen and coworkers reported on the synthesis of poly(trans-hexahydro-2(3H)-benzofuranone), which displayed high stress at break (σ=55 MPa), high Tm values (≥126° C.), and good thermal stability (T_{d}=340° C.). Helms and coworkers synthesized cross-linked polymers comprising diketoenamine linkages, which were depolymerized in the presence of mixed plastic waste. Moore and coworkers developed a polymerization strategy for cyclic disulfides that yielded high molar mass polydisulfides (Mn up to 630 kDa), which could be depolymerized to monomer with 95% purity. Despite great progress in chemical recycling, persistent challenges include expensive and difficult monomer syntheses, inefficient polymer syntheses with costly catalysts often requiring low temperatures, impractical thermomechanical properties, and energy-intensive depolymerization to monomer. Together, these factors have limited the wide-spread use of chemically recyclable polymers.

Cyclic methylene acetals and their corresponding polyacetals have long been studied for their rapid polymerization and acid-catalyzed degradation. Although mono- and disubstituted acetals are well-known to undergo facile degradation under mildly acidic conditions (and are therefore commonly used as diol protecting group, methylene acetals are quite stable except under strongly acidic conditions. For this reason, polyoxymethylene (POM), is widely used for its desirable polyolefin-like properties (T_{g}<0° C., Tₘ>100° C.), chemical resistance, and excellent wear properties. To address these challenges, we identified polyacetals as promising candidates for materials capable of CRM given their moderate ceiling temperatures, good thermal and chemical stability, and selective acid-catalyzed depolymerization to cyclic acetal monomer. Furthermore, a wide range of cyclic acetal monomers are either commercially available or can be readily synthesized from diols and formaldehyde on large scales.

A key challenge to commercial application of polyacetals capable of CRM is obtaining materials of suitably high molecular weights to possess useful mechanical properties. Polyacetals are commonly synthesized by uncontrolled cationic ring-opening polymerization (CROP) of cyclic acetal monomers using Bronsted or Lewis acid catalysts (FIG. 1A). The uncontrolled nature of these polymerization methods affords low molecular weight polyacetals with poor mechanical properties. Improving molecular weight control during CROP of cyclic acetals is therefore imperative to achieve high molecular weight polyacetals with viable thermomechanical properties.

### SUMMARY OF THE DISCLOSURE

In an aspect, the present disclosure provides poly(cyclic acetal)s. In various examples, the poly(cyclic acetal)s are homopolymers and copolymers. In various examples, a poly(cyclic acetal) comprises: a number-average molecular weight (Mₙ) of from about 10 kiloDaltons (kDa) to about 3000 kDa, including all integer kDa values and ranges therebetween. In various examples, a poly(cyclic acetal) comprises polymer chain ends that are not hydroxyl groups. In various examples, the polymer chain ends are independently chosen at each occurrence from alkyl ether groups, aryl ether groups, alkylaryl ether groups, and the like. In various examples, the poly(cyclic acetal) comprises one or more homopolymer(s) or copolymer(s) of poly(1,3-dioxepane)(PDXP), poly(1,3-dioxocane) (PDXC), poly(1,3,6-trioxocane) (PTXC), poly(1,3-hexahydrobenzodioxole) (PHBD), poly(1,3-dioxolane) (PDXL), or the like, or any combination thereof.

In an aspect, the present disclosure provides articles of manufacture comprising one or more poly(cyclic acetal(s)) of the present disclosure. In various examples, the article is a spun article, a molded article, an extruded article, a pultruded article, a cast article, a blown article, a woven article, a 3D printed article, or the like.

In an aspect, the present disclosure provides methods for polymerizing one or more of the poly(cyclic acetal)s. In various examples, a polymerization method comprises: combining: one or more cyclic acetal monomer(s); one or more haloalkyl ether initiator(s); and one or more Lewis acid catalyst(s), to form a polymerization mixture, wherein a poly(cyclic acetal) is formed. In various examples, the cyclic acetal monomer(s) is/are cyclic methylene acetal monomer(s) chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and the like, and any combination thereof. A polymerization mixture can comprise various types and amounts of initiator(s). In various examples, the initiator(s) is/are haloalkyl ether initiator(s). In various examples, the Lewis acid catalyst(s) is/are Lewis acid(s). In various examples, the Lewis acid catalyst(s) is/are of the form MXₙ, wherein M=Ga, In, Zn, Sb^{V}, Sn^{IV}, or Fe, and wherein X=Cl, Br, I or OTf. In various examples, the polymerization mixture further comprises one or more proton trap(s). In various examples, the proton trap(s) is/are sterically hindered base(s).

In various examples, a method comprises: combining: one or more cyclic acetal monomer(s); one or more organic cation salt catalyst(s); and one or more proton trap(s), to form a polymerization mixture, wherein a poly(cyclic acetal) is formed. In various examples, the cyclic acetal monomer(s) is/are cyclic methylene acetal monomer(s) chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and the like, and any combination thereof. In various examples, the organic cation salt catalyst(s) is/are of the form C⁺A⁻, wherein C⁺ is chosen from electrophilic alkylating agents, and the like, and A⁻ is chosen from non-nucleophilic anions, complex anions, non-complex anions, and the like, and any combination thereof. In various examples, the polymerization mixture further comprises one or more chain transfer agent(s) (CTA(s)).

In an aspect, the present disclosure provides methods of depolymerizing one or more of the poly(cyclic acetal)(s) of the present disclosure. In various examples, a depolymerization method comprises: combining: one or more poly(cyclic acetal(s)); and one or more acid catalyst(s) (e.g., acid catalyst(s) having a pKₐ of less than or equal to 4), to form a depolymerization mixture; and heating the depolymerization mixture, to form the cyclic acetal(s). In various examples, the method further comprises removing or isolating the cyclic acetal(s) from the depolymerization mixture.

### BRIEF DESCRIPTION OF THE FIGURES

For a fuller understanding of the nature and objects of the disclosure, reference should be made to the following detailed description taken in conjunction with the accompanying figures herein.

FIGS. 1A-1B show: (FIG. 1A) uncontrolled cationic ring-opening polymerization (CROP) methods of synthesizing polyacetals using Bronsted or Lewis acid initiators (H⁺-catalyzed CROP), leading to low molecular weight polymers with poor thermomechanical properties. (FIG. 1B) Controlled reversible-deactivation CROP of cyclic acetals and general monomer synthesis. Reversible-deactivation imparts control.

FIGS. 2A-2E show: (FIG. 2A) InBr₃/MOMBr-catalyzed RD-CROP catalytic cycle of cyclic acetals. (FIG. 2B) ¹H NMR spectra acquired for DXL variable temperature NMR measurement of [DXL]_{eq} vs. temperature. (FIG. 2C) Effect of InX₃/MOMX halide composition on polymerization rate of DXL. (FIG. 2D) Linear M_{n,GPC} versus [M]₀: [MOMBr]₀ plots for each cyclic acetal derivative are characteristic of living polymerization behavior, [DXL]₀=8 M, [DXP]₀=5 M, [DXC]₀=5 M, [TXC]₀=3 M, [HBD]₀=5 M. (FIG. 2E) Sequential DXL monomer addition during polymerization confirms living chain end retention.

FIGS. 3A-3C show tensile testing data and images of PDXL. (FIG. 3A) PDXL exhibits comparable mechanical properties to commercially available commodity polymers. (FIG. 3B) Image of colorless, semi-crystalline PDXL. (FIG. 3C) Stability of PDXL towards various acids and bases, PDXL samples were doped with a variety of acid or base additives possessing a range of pKₐ and pK_{b} values at 5 mol % (8-15 wt. %) loading.

FIGS. 4A-4D show: (FIG. 4A) Reaction scheme showing controlled polymerization of DXL to PDXL and triggered depolymerization of PDXL enabling chemical recycling back to monomer. (FIG. 4B) Neat PDXL is thermally stable to T_{d,5%}=353° C., but can be readily depolymerized in the presence of 5 mol % strong acids including camphorsulfonic acid (CSA) and diphenylphosphoric acid (DPP). (FIG. 4C) PDXL was doped with 2 mol % camphorsulfonic acid and depolymerized via distillation at 140° C. over 80 minutes. Monomer feedstocks with only minor CSA impurities were recovered in 97 wt % yield. (FIG. 4D) Distillate containing pure DXL monomer can be recovered from mixed polymer feedstocks in 77 wt. % yield in the presence of an acidic resin (DOWEX-50). The commodity materials were obtained from recycling facilities and low molecular weight PEG was added as a viscosity modifier to aid in stirring.

FIG. 5 shows a MALDI-TOF spectrum and end-group analysis of PDXL (M_{n,GPC}=4.1 kDA, Ð=1.42) synthesized using the InBr₃/MOMBr catalyst system and quenched with NaOBn. *Exp m/z could not be determined due to overlap with MOM-PDXL-OBn peak.

FIG. 6 shows a MALDI-TOF spectrum and end-group analysis of PDXP (M_{n,GPC}=3.6 kDa, Ð=1.45) synthesized using the InBr₃/MOMBr catalyst system and quenched with NaOBn.

FIG. 7 shows a MALDI-TOF spectrum and end group analysis of PDXC (M_{n,GPC}=3.8 kDa, Ð=1.43) synthesized using the InBr₃/MOMBr catalyst system and quenched with NaOBn.

FIG. 8 shows a MALDI-TOF spectrum and end-group analysis of PTXC (M_{n,GPC}=4.8 kDa, Ð=1.51) synthesized using the InBr₃/MOMBr catalyst system and quenched with NaOBn.

FIG. 9 shows a VT-NMR temperature calibration using the MeOH thermometer method.

FIG. 10 shows a ¹H NMR spectra of InBr₃/MOMBr-catalyzed RD-CROP of DXL in CD₂Cl₂ obtained using VT-NMR spectroscopy from −30 to 40° C. p-Xylene was used as an internal standard. [DXL]₀=4.0 M.

FIG. 11 shows a plot of R·ln([M]_{eq}) vs. 1/T for DXL obtained by VT-NMR.

FIG. 12 shows an image of high molecular weight PDXL sample directly after precipitation into cold diethyl ether.

FIG. 13 shows GPC traces of 111 kDa PDXL before (black: M_{n,GPC}=99.3 kDa, Ð=1.75) and after being placed in a vial in a dry (left/dashed: M_{n,GPC}=90.9 kDa, Ð=1.89) or humid (right/dashed: M_{n,GPC}=94.1 kDa, Ð=1.84) environment at 57° C. for 7 days. In both cases, no changes were observed.

FIG. 14 shows images of high molecular weight PDXL dogbones after molding in a hot press at 90° C. for 20 min and naturally cooling to room temperature.

FIG. 15 shows images of low molecular weight 37.9 kDa and 59.9 kDa PDXL demonstrating the brittle or poor mechanical properties observed for low molar mass materials below the entanglement threshold.

FIG. 16 shows images of high molecular weight PDXL just before and just after reaching the stress at break fracture during tensile testing. Due to strain-induced crystallization, the sample stretches with nonuniform widths. After fracture, the samples display frayed ends and crimps down the gauge length due to crystallization.

FIG. 17 shows an image of high molecular weight PDXL sample after tensile testing demonstrating the frayed edges, crimping along the gauge length, and difference in opacity from the crystallized mid-section to noncrystallized edges.

FIG. 18 shows an image of high molecular weight PDXL sample during tensile testing at the onset of strain induced crystallization. The edge of the semi-crystalline area is denoted with white arrows.

FIG. 19 shows DSC traces of PDXL from the first heat from a sample (111 kDa) before any applied strain (bottom) and after undergoing strain-induced crystallization in a tensile tester (middle) as compared to the second heating ramp (top). All samples were measured at a heating rate of 10° C./min.

FIG. 20 shows isothermal TGA plots of PDXL (127 kDa) with 2 mol % CSA measured at 100, 110, 120, 130, and 140° C. Heat was ramped to the given isothermal temperature at 50° C./min, then maintained over 10-100 minutes until remaining mass reached a plateau at 7%. Linear degradation data was taken starting at 90% to account for initial temperature equilibration time and stopped at 70% to ensure rate was extracted from a linear degradation regime.

FIG. 21 shows Arrhenius activation energy (E_{A}) calculated from the linear isothermal degradation rate of PDXL with 2 mol % CSA at 140, 130, 120, 110, and 100° C. from 90 to 70% mass remaining. The rate was calculated as the % mass loss per second.

FIGS. 22A-22D show images of PDXL before, during, and after depolymerization with 2 mol % added camphorsulfonic acid via distillation at 140° C. (FIG. 22A) Pristine PDXL. (FIG. 22B) PDXL with CSA. (FIG. 22C) Distillation at 140° C. (FIG. 22D) Recovered DXL monomer.

FIG. 23 shows images of 84.4 kDa PDXL tensile specimen synthesized from after repolymerization from recovered DXL feedstock.

FIG. 24 shows images of PDXL/HDPE/LDPE/PS samples before depolymerization and pure DXL recovered after depolymerization with Dowex-50 for 2 hours at 140° C.

FIGS. 25A-25D show images of PDXL before, during, and after depolymerization with 20 wt. % added Dowex-50 via distillation at 140° C. (FIG. 25A) Commodity mixture. (FIG. 25B) Mixture with acid resin. (FIG. 25C) Distillation at 140° C. (FIG. 25D) Recovered DXL monomer.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise stated, “about,” “approximately,” “substantially,” or the like, when used in connection with a measurable variable such as, for example, a parameter, an amount, a temporal duration, or the like, are meant to encompass variations of, for example, a specified value including, for example, those within experimental error (which can be determined by for example, a given data set, an art accepted standard, and/or with a given confidence interval (e.g. 90%, 95%, or more confidence interval from the mean), such as, for example, variations of +/−10% or less, +/−5% or less, +/−1% or less, and +/−0.1% or less of and from the specified value), insofar such variations are appropriate to perform in the context of the disclosure. As used herein, unless otherwise stated, the terms “about,” “approximate,” “at or about,” and “substantially” can mean that the amount or value in question can be the exact value or a value that provides equivalent results or effects as recited in the sample claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error, and the like, and other factors known to those of skill in the art such that, for example, equivalent results, effects, or the like are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also, unless otherwise stated, include individual values (e.g., about 1%, about 2%, about 3%, and about 4%) and the sub-ranges (e.g., about 0.5% to about 1.1%; about 0.5% to about 2.4%; about 0.5% to about 3.2%, and about 0.5% to about 4.4%, and other possible sub-ranges) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, a strong-acid is an acid with a pKₐ less than or equal to 4.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

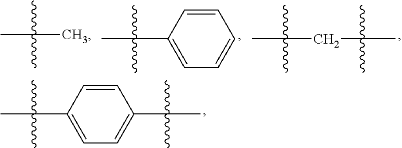

and the like.

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is C₁ to C₂₅, including all integer numbers of carbons and ranges of numbers of carbons therebetween (e.g., C₁, C₂, C₃, C₄, C₅, C₆, 20 C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, and C₂₅). The alkyl group may be unsubstituted or substituted with one or more substituent(s). Examples of substituents include, but are not limited to, various substituents such as, for example, halogens (-F, -Cl, -Br, and -I), aliphatic groups (e.g., alkyl groups, alkenyl groups, alkynyl groups, and the like), aryl groups, alkoxide groups, carboxylate groups, carboxylic acids, ether groups, amine groups, and the like, and any combination thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused ring, biaryl groups, or a combination thereof. The aryl group may be unsubstituted or substituted with one or more substituent.

Examples of substituents include, but are not limited to, substituents such as, for example, halogens (-F, -Cl, -Br, and -I), aliphatic groups (e.g., alkyl groups, alkenyl groups, alkynyl groups, and the like), aryl groups, alkoxides, 5 carboxylates, carboxylic acids, ether groups, and the like, and combinations thereof. Aryl groups may contain hetero atoms, such as, for example, nitrogen (e.g., pyridinyl groups and the like). Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

In an aspect, the present disclosure provides poly(cyclic acetal)s. In various examples, a poly(cyclic acetal) is a homopolymers or a copolymer (e.g., a poly(cyclic acetal) copolymer or the like). Non-limiting examples of poly(cyclic acetal)s are provided herein (e.g., in the Statements and the Examples). In various examples, a poly(cyclic acetal) is made by a method of the present disclosure.

A poly(cyclic acetal) can comprise various chemical structures. Methods of determining chemical structure are known in the art. Non-limiting examples of methods of determining chemical structure include ultraviolet-visible (UV) spectroscopy, infrared (IR) spectroscopy, Raman spectroscopy, nuclear magnetic resonance (NMR) spectroscopy, electron spin resonance (ESR) spectroscopy, X-ray diffraction (XRD), mass spectrometry (MS), and the like. In various examples, a poly(cyclic acetal) comprises a polymer backbone having one or more oxyalkylene repeat group(s), or the like. In various examples, a poly(cyclic acetal) is a poly(cyclic methylene acetal), or the like. In various examples, a poly(cyclic methylene acetal) comprises a polymer backbone having one or more oxymethylene-a/t-oxyalkylene repeat group(s), or the like. In various examples, a poly(cyclic acetal) comprises a polymer backbone excluding one or more cyclic acetal repeat group(s) within and/or pendant from the polymer backbone, or the like.

A poly(cyclic acetal) can comprise (or be) various poly(cyclic acetal) homopolymers and copolymers. A poly(cyclic acetal) can comprise (or be) various poly(cyclic acetal) copolymers which can be compositionally different, structurally different, different in molecular weight, or the like, or a combination thereof). In various examples, a poly(cyclic acetal) comprises (or is) various random copolymers, alternating copolymers, diblock copolymers, multiblock copolymers, or the like, or combinations thereof. In various examples, a poly(cyclic acetal) comprises one or more homopolymer(s) or copolymer(s) of poly(1,3-dioxepane)(PDXP), poly(1,3-dioxocane) (PDXC), poly(1,3,6-trioxocane) (PTXC), poly(1,3-hexahydrobenzodioxole) (PHBD), poly(1,3-dioxolane) (PDXL), or the like, or any combination thereof.

A poly(cyclic acetal) can comprise various molecular mass values. Methods of determining molecular mass are known in the art. Non-limiting examples of determining molecular mass include gel permeation chromatography (GPC), GPC with multi-angle light scattering (MALS), GPC/MALS combined with UV absorption and differential refractometry, low-angle laser light scattering (LALLS), viscometry, Analytical Temperature Rising Elution Fractionation (ATREF) techniques, and the like). In various examples, a poly(cyclic acetal) comprises a number-average molecular weight (Mₙ) value of about 10 kiloDaltons (kDa) to about 3000 kDa (e.g., from about 50 kDA to about 2000 kDa, from about 50 kDA to about 3000 kDa, from about 75 kDa to about 200 kDa, from about 75 kDa to about 1000 kDa, from about 100 kDa to about 1000 kDa, from about 100 kDa to about 500 kDa, or from about 200 kDa to about 1000 kDa), including all integer kDa values and ranges therebetween. In various examples, a poly(cyclic acetal) comprises a Mn value of about 10 kDa or greater, 20 kDa or greater, about 30 kDa or greater, about 40 kDa or greater, about 50 kDa or greater, about 60 kDa or greater, about 70 kDa or greater, about 80 kDa or greater, about 90 kDa or greater, about 100 kDa or greater, about 110 kDa or greater, about 120 kDa or greater, about 130 kDa or greater, about 140 kDa or greater, about 150 kDa or greater, about 160 kDa or greater, about 170 kDa or greater, about 180 kDa or greater, about 190 kDa or greater, or about 200 kDa. In various examples, a poly(cyclic acetal) comprises a Mn value of about 200 kDa or greater, about 300 kDa or greater, about 400 kDa or greater, about 500 kDa or greater, about 600 kDa or greater, about 700 kDa or greater, about 800 kDa or greater, about 900 kDa or greater, about 1000 kDa, about 1100 kDa, about 1200 kDa or greater, about 1300 kDa or greater, about 1400 kDa or greater, about 1500 kDa or greater, about 1600 kDa or greater, about 1700 kDa or greater, about 1800 kDa or greater, about 1900 kDa or greater, about 2000 kDa, about 2100 kDa, about 2200 kDa or greater, about 2300 kDa or greater, about 2400 kDa or greater, about 2500 kDa or greater, about 2600 kDa or greater, about 2700 kDa or greater, about 2800 kDa or greater, about 2900 kDa or greater, or about 3000 kDa.

A poly(cyclic acetal) can comprise various dispersity (Ð) values. In various examples, a poly(cyclic acetal) comprises a dispersity (Ð) value of from about 1.3 to about 5 (e.g., from about 1.5 to about 1.8), including all 0.01 values and ranges therebetween.

A poly(cyclic acetal) can comprise various chain end groups. Methods of chain end analysis are known in the art. Non-limiting examples of chain end analysis include NMR, mass spectrometry, IR spectroscopy, Raman spectrometry, and the like). In various examples, a poly(acetal) comprises chain ends which are not hydroxyl groups. In various examples, all of the chain ends of a poly(cyclic acetal) are not hydroxyl groups. In various examples, over about 50% of the chain ends of a poly(cyclic acetal) are not hydroxyl groups (e.g., over about 60%, over about 70%, over about 80%, over about 90%, over about 99%, or substantially all). In various examples, over about 50% of the chain ends of a poly(cyclic acetal) are not hydroxyl groups in an as-formed poly(cyclic acetal). In various examples, over about 50% of the chain ends of a poly(cyclic acetal) are not hydroxyl groups after quenching of an as-formed poly(cyclic acetal). In various examples, over 50% of the chain ends of a poly(cyclic acetal) are not hydroxyl groups after chemical modification of the chain ends of an as-formed and/or quenched poly(cyclic acetal).

In various examples, a poly(cyclic acetal) comprises chain ends independently chosen at each occurrence from alkyl ether groups, aryl ether groups, alkylaryl ether groups, heterocyclic groups, halide groups, triflate groups, nucleophilic groups such as, for example, amine groups, thiol groups, phosphine groups, or the like), and the like. In various examples, a poly(cyclic acetal) does not comprise chain ends independently chosen at each occurrence from initiator-derived end groups, catalyst-derived end groups (e.g., heteropolyacid catalyst-derived end groups, or the like), or the like. In various examples, .

A poly(cyclic acetal) can exhibit various thermochemical stabilities. Methods of measuring thermal degradation are known in the art. Non-limiting examples of methods of measuring thermal degradation include Dynamic or Isothermal Thermogravimetric (TGA) Analysis, or the like. In various examples, a poly(cyclic acetal) has a thermal stability (T_{d,5%}) in the absence of additives of from about 337° C. to about 392° C., including all 0.1° C. values and ranges therebetween. In various examples, a poly(cyclic acetal) comprises a thermal stability (T_{d,50%}) of from about 377° C. to about 462° C., including all 0.1° C. values and ranges therebetween, with or without 2 mol % of one or more additive(s) having a pKₐ greater than 4. In various examples, additive(s) having a pKₐ greater than 4 are chosen from acids, alcohols, amines, and any combination thereof. In various examples, a poly(cyclic acetal) comprises an Arrhenius activation energy (Eₐ) of about 85.0 kJ/mol, including all 0.01 kJ/mol values and ranges therebetween, with 2 mol % of one or more acid(s) having a pKₐ less than or equal to 4.

A poly(cyclic acetal) can exhibit various thermal properties. Methods of measuring thermal transitions are known in the art. Non-limiting examples of methods of measuring thermal transitions include Differential Scanning calorimetry (DSC), and the like). In various examples, a poly(cyclic acetal) is semicrystalline. In various examples, a poly(cyclic acetal) has a melting temperature (Tₘ) of from about 0° C. to about 120° C., including all 0.1° C. values and ranges therebetween. In various examples, a poly(cyclic acetal) has a Tₘ of about 0° C. or greater, about 10° C. or greater, about 20° C. or greater, about 30° C. or greater, about 40° C. or greater, about 50° C. or greater, about 60° C. or greater, about 70° C. or greater, about 80° C. or greater about 90° C. or greater, about 100° C. or greater, about 110° C. or greater, or about 120° C. In various examples, a poly(cyclic acetal) comprises a glass transition temperature (T_{g}) of from about −70° C. to about 50° C., including all 0.1° C. values and ranges therebetween. In various examples, a poly(cyclic acetal) has a T_{g} of about −70° C. or greater, about −60° C. or greater, about −50° C. or greater, about −40° C. or greater, about −30° C. or greater, about −20° C. or greater, about −10° C. or greater, about 0° C. or greater, about 10° C. or greater about 20° C. or greater, about 30° C. or greater, about 40° C. or greater, or about 50° C.

A poly(cyclic acetal) can comprise various tensile properties. Methods of measuring thermomechanical properties are known in the art. Non-limiting examples of methods of measuring thermomechanical properties include Tensile Testing, or the like). In various examples, a poly(cyclic acetal) is a thermoplastic. In various examples, a poly(cyclic acetal) comprises a tensile stress at break of about 10 MPa to about at least 50 MPa, including all integer MPa values and ranges therebetween. In various examples, a poly(cyclic acetal) comprises a tensile stress at break of about 10 MPa or greater, about 20 MPa or greater, about 30 MPa or greater, about 40 mPa or greater, or about 50 MPa. In various examples, a poly(cyclic acetal) comprises a tensile strain at break of from about 1% to about 800%, including all integer % values and ranges therebetween. In various examples, a poly(cyclic acetal) comprises a tensile strain at break of about 5% or greater, about 50% or greater, about 100% or greater, about 200% or greater, about 300% or greater, about 400% or greater, about 500% or greater, about 600% or greater, about 700% or greater, or about 800%.

A poly(cyclic acetal) can comprise various depolymerization susceptibilities. In various examples, a poly(cyclic acetal) is chemically depolymerized into said cyclic acetal by one or more acid catalyst(s) having a pKₐ of less than or equal to 4, or the like.

In various examples, a poly(cyclic acetal) comprises one or more or all of the following: a thermal stability (T_{d,5%}) of from about 337° C. to about 392° C.; a thermal stability (T_{d,50%}) of from about 377° C. to about 462° C., with or without 2 mol % of one or more additive(s) having a pKₐ greater than 4; an Arrhenius activation energy (Eₐ) of about 85.0 kJ/mol, with 2 mol % of one or more acid(s) having a pKₐ of less than or equal to than 4; a melting temperature (Tₘ) of from about 0° C. to about 120° C.; a glass transition temperature (T_{g}) of from about −100° C. to about 100° C.; a tensile stress at break about 10 MPa to about 50 MPa; or a tensile strain at break of from about 1% to about 800.

In various examples, a poly(cyclic acetal) is a poly(1,3-dioxolane) (PDXL). In various examples, the PDXL comprises a Mₙ value of from about 10 kiloDaltons (kDa) to about 3000 kDa, including all integer kDa values and ranges therebetween. In various examples, the PDXL comprises a dispersity (Ð) value of from about 1.3 to about 5 (e.g., from about 1.5 to about 1.8), including all 0.01 values and ranges therebetween. In varous examples, the PDXL comprises chain ends which are not hydroxyl groups. In various examples, all of the chain ends are not hydroxyl groups. In various examples, over about 50% of the chain ends of the PDXL are not hydroxyl groups (e.g., over about 60%, over about 70%, over about 80%, over about 90%, over about 99%, or substantially all). In various examples, the PDXL comprises a Mₙ value of from about 10 kiloDaltons (kDa) to about 3000 kDa, and over about 50% of the chain ends of the poly(cyclic acetal) are not hydroxyl groups. In various examples, the PDXL comprises chain ends independently chosen at each occurrence from alkyl ether groups, aryl ether groups, alkylaryl ether groups, heterocyclic groups, halide groups, triflate groups, nucleophilic groups such as, for example, amine groups, thiol groups, phosphine groups, or the like), and the like. In various examples, the PDXL does not comprise chain ends independently chosen at each occurrence from initiator-derived end groups, catalyst-derived end groups (e.g., heteropolyacid catalyst-derived end groups, or the like), or the like. In various examples, the molecular weight (Mₙ) of the PDXL remains unchanged by soaking in neutral water for about 1 day, about 2 days, about 3 days, about 4 days, about 5 days, about 6 days, about 1 week, about 2 weeks, about 3 weeks, about 1 month, about 2 months, about 3 months, about 100 days, or greater. In various examples, the molecular weight (Mₙ) of the PDXL remains unchanged after exposure to either dry or humid conditions at 57° C. for about 1 day, about 2 days, about 3 days, about 4 days, about 5 days, about 6 days, about 1 week, or greater. In various examples, the PDXL comprises one or more or all of the following: a thermal stability (T_{d,5%}) of from about 337° C. to about 392° C.; a thermal stability (T_{d,50%}) of from about 377° C. to about 462° C., with or without 2 mol % of one or more additive(s) having a pKₐ greater than 4; an Arrhenius activation energy (Eₐ) of about 85.0 kJ/mol, with 2 mol % of one or more acid(s) having a pKₐ less than or equal to 4; a melting temperature (Tₘ) of from about 0° C. to about 120° C.; a glass transition temperature (T_{g}) of from about −100° C. to about 100° C.; a tensile stress at break about 10 MPa to about 50 MPa; or a tensile strain at break of from about 1% to about 800.

A poly(cyclic acetal) can have various forms. In various examples, a poly(cyclic acetal) is in the form of a solution, an emulsion, a slurry, a dispersion, a particle, a flake, a pellet, a powder, a granule, a tube, a sphere, a fiber, a foam, a film, a textile, a mesh, a sheet, a bar, a monolith, or the like. In various examples, the poly(cyclic acetal) is in the form of a packaging film.

In an aspect, the present disclosure provide articles of manufacture comprising one or more poly(cyclic acetal(s)) of the present disclosure. Non-limiting examples of articles of manufacture are provided herein (e.g., in the Statements and the Examples).

In various examples, an article of manufacture is a spun article, a molded article, an extruded article, a coated article, a blown article, a woven article, a drawn article, a laminated article, or a 3D printed article, or the like. Articles of manufacture can be used in various applications. In various examples, an article of manufacture is used in packaging, a consumer products, biomedical devices, industrial products, or pharmaceutical compositions. In various examples, an article is a packaging film. In various examples, an article is an adhesive.

In an aspect, the present disclosure provides methods of polymerizing one or more poly(cyclic acetal)s. Non-limiting examples of methods of preparing poly(cyclic acetal)s are provided herein (e.g., in the Statements and the Examples). In various examples, a poly(cyclic acetal) is prepared by a method of the present disclosure and, optionally, comprises one or more feature(s) of a poly(cyclic acetal) of the present disclosure.

In various examples, a polymerization method for polymerizing one or more poly(cyclic acetal)s comprises combining: one or more cyclic acetal monomer(s), one or more polymerization initiator(s), and one or more polymerization catalyst(s), to form a polymerization mixture, wherein a poly(cyclic acetal) is formed. A method can use various polymerization reactions to form a poly(cyclic acetal). In various examples, a method uses a ring opening polymerization reaction, or the like.

A method can use various cyclic acetal monomer(s). In various examples, cyclic acetal monomer(s) is/are cyclic methylene acetal monomer(s), or the like. In various examples, cyclic methylene acetal monomer(s) is/are chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and the like, and any combination thereof. In various examples, cyclic acetal monomer(s) is/are present in a polymerization mixture at from about 1.5M to about 15M, including all integer M values and ranges therebetween. In various examples, cyclic acetal monomer(s) is/are present in a polymerization mixture at about 1.5 M or greater, about 2 M or greater, about 3 M or greater, about 4 M or greater, about 5 M or greater, about 6 M or greater, at about 7 M or greater, at about 8 M or greater, at about 9 M or greater, about 10 M or greater, at about 11 M or greater, at about 12 M or greater, at about 13 M or greater, at about 14 M or greater, or at about 15 M. In various examples, cyclic acetal monomer(s) is/are present neat (e.g., in the absence of one or more solvent(s)).

A method can use various polymerization initiator(s). In various examples, polymerization initiator(s) is/are halogen-terminated compounds, triflate-terminated compound(s), or the like, or any combination thereof. In various examples, the halogen-terminated compound(s) is/are haloalkyl ether initiator(s), or the like. In various examples, haloalkyl ether initiator(s) comprise(s) a C₁-C₂₅ alkyl group chosen from linear, cyclic, branched, substituted alkyl (e.g., arylalkyl or the like), unsaturated, and saturated C₁-C₂₅ alkyl group(s), and any combination thereof. In various examples, haloalkyl ether initiator(s) is/are chosen from benzyl chloromethyl ether, bromomethyl methyl ether, 1-chloromethyl adamantane, chloromethyl cyclohexyl ether, chloromethyl dococosyl ether, chloromethyl ethyl ether, chloromethyl methyl ether, tert-butyl chloromethyl ether, methoxyethyl chloromethyl ether, and the like, and any combination thereof.

In various examples, haloalkyl ether initiator(s) is/are present in a polymerization mixture at 1×10⁻⁶ mM to 1 mM, including all integer mM values and ranges therebetween. In various examples, an initial molar ratio of cyclic acetal monomer(s) to haloalkyl ether initiator(s) is from about 200:1 to about 1600:1, including all integer molar ratio values and ranges therebetween. In various examples, an initial molar ratio of cyclic acetal monomer(s) to haloalkyl ether initiator(s) is about 200:1 or greater, about 300:1 or greater, about 400:1 or greater, about 500:1 or greater, about 600:1 or greater, about 700:1 or greater, about 800:1 or greater, about 900:1 or greater, about 1000:1 or greater, about 1100:1 or greater, about 1200:1 or greater, about 1300:1 or greater, about 1400:1 or greater, about 1500:1 or greater, or about 1600:1.

In various examples, polymerization initiator(s) is/are not chosen from: a non-metal halide capable of affording sulfonium cations or phosphonium cations, such as, for example, thionyl chloride, sulfuryl chloride, sulfonic acid halides, benzene sulfuryl chloride, phosphorous oxychloride, phosphorus trichloride, phosphorus pentachloride, (CH₃)₄PCl, C₆H₅POCl₂, or the like, or any combination thereof; an oxygen containing heterocyclic compound such as, for example, alkylene oxides (e.g., epichlorohydrin, epibromohydrin, epifluorohydrin, epiiodohydrin, ethylene oxide, propylene oxide, styrene oxide, butadiene monoxide, 2,3-epoxybutane, or the like, or any combination thereof), styrene oxide, ethylene carbonate, lactones, or the like, or any combination thereof; radical initiators, such as, for example, organic peroxides (e.g., benzoyl peroxide, aromatic-substituted benzoyl peroxide, lauroyl peroxide, acetyl peroxide, ketone peroxide, cumene hydroperoxide, t-butyl hydroperoxide, di-t-butyl peroxide, or the like, or any combination thereof), sulfinic acid, aromatic sulfinic acid, or the like, or any combination thereof; 1-alkanoic acid, or the like; a,a′-azobisisobutyronitrile, or the like; acid anhydrides, acid chlorides, acid esters, or the like (e.g., acetic anhydride, acetyl chloride, benzoyl chloride, or the like, or any combination thereof), or any combination thereof; strong acid esters, such as, for example, dialkylsulfates, aryl sulphonates, or the like, or any combination thereof; alkyl halides and aryl halides, such as, for example, t-butyl chloride, benzyl chloride, benzotrichloride, or the like; ketene, diketene, alkylketene, dialkylketene, arylketene, diarylketene, or the like, or any combination thereof; azo and diazo compounds, such as, for example, diazomethane, or the like, or any combination thereof; superstrong acids, such as, for example, heteropolyacids (e.g., acids having a Keggin structure in which a central atom of a framework acid is an atom chosen from molybdenum, tungsten, vanadium, and the like and in which a heteroatom is an atom chosen from silicon, phosphorus, germanium, titanium, zirconium, boron, arsenic, cobalt, and the like, such as, for example: phosphotungstic acid, silicotungstic add, phosphomolybdic acid, silicomolybdic acid, borotungstic acid, boromolybdic acid, cobaltomolybdic acid, cobaltotungstic acid, arsenotungstic acid, germanotungstic acid, phosphomolybdotungstic acid, and boromolybdotungstic acid, or the like, or any combination thereof), isopolyacids, perfluoroalkylsulfonic acids, or the like, or any combination thereof; oxygen-containing organic compounds, such as, for example, ethers (e.g., diethyl ether, ethylene glycol dimethyl ether, diethylene glycol, dimethyl ether, 1,4-dioxane, or the like, or any combination thereof), carbonyl compounds (e.g., acetone, methyl ethyl ketone, methyl isobutyl ketone, diacetone alcohol, acetyl acetone, ethyl acetate, butyl acetate, methyl propionate, cyclohexanone, or the like, or any combination thereof), or the like, or any combination thereof; triarylcarbonium salts, or the like; and trialkyloxonium salts, or the like.

A method can use various polymerization catalyst(s). In various examples, polymerization catalyst(s) is/are Lewis acid catalyst(s), or the like. In various examples, Lewis acid catalyst(s) is/are Lewis acid(s) of the form MXₙ. In various examples, M is chosen from Ga, In, Zn, Sb^{V}, Sn^{IV}, and Fe, and X is chosen from Cl, Br, I, and OTf. In various examples, M is chosen from Ga^{III}, In^{III}, Zn^{II}, Sb^{V}, Sn^{IV}, and Fe^{III}. In various examples, M=In (e.g., In^{III}, or the like), Zn (e.g., Zn^{II}, or the like), or Sb^{V}. In various examples, Lewis acid catalyst(s) is/are chosen from InCl₃, InBr₃, InI₃, In(OTf)₃, ZnCl₂, ZnBr₂, ZnI₂, Zn(OTf)₂, and any combination thereof. In various examples, M is not chosen from B, Al, Ga, Sb^{III}/Sb^{V}, Bi, Fe, Ti, Zr, and Hf. In various examples, Lewis acid catalyst(s) is/are present in a polymerization mixture at 1×10⁻⁵ mM to 2 mM, including all integer M values and ranges therebetween. In various examples, an initial molar ratio of cyclic acetal monomer(s) to Lewis acid catalyst(s) is from about 200:1 to about 1600:1, including all integer molar ratio values and ranges therebetween.

In various examples, a polymerization mixture further comprises one or more proton trap(s). In various examples, proton trap(s) is/are sterically hindered base(s), or the like. In various examples, sterically hindered base(s) is/are chosen from 2,6-di-tert-butylpyridine, 2,6-di-tert-butyl-4-methylpyridine, 2,4,6-tri-tert-butylpyridine, 2,4,6-tri-tert-butylprimidine, and the like, and any combination thereof. In various examples, proton trap(s) is/are present in a polymerization mixture at from about 2×10⁻⁵ mM to about 1 mM, including all integer M values and ranges therebetween. In various examples, an initial molar ratio of cyclic acetal monomer(s) to proton trap(s) is from about 200:1 to about 1600:1, including all integer molar ratio values and ranges therebetween.

In various examples, a polymerization mixture further comprises one or more solvent(s), or the like. In various examples, the one or more solvent(s) is/are chosen from halocarbon solvents (e.g., dichloromethane, chloroform, or the like, or any combination thereof), aryl solvents (e.g., toluene, xylene, or the like), ether solvents, and the like, any combination thereof.

In various examples, the solvent(s) is/are chosen from dichloromethane, toluene, chloroform, and the like, and any combination thereof.

A polymerization reaction can be performed under various conditions. In various examples, a polymerization reaction is carried out at a temperature of from about −90° C. to about 50° C., including all 0.1° C. values and ranges therebetween. In various examples, a polymerization reaction is carried out at a temperature of about 25° C. Methods of determining monomer to polymer conversion are known in the art. Non-limiting examples of determining monomer to polymer conversion include NMR spectroscopy (e.g., ¹H NMR spectroscopy, ¹³C spectroscopy, and the like, and any combination thereof), and the like). In various examples, a polymerization reaction is carried out until reaching a monomer to polymer conversion of from about 20% to about 100% conversion, including all integer % values and ranges therebetween.

A polymerization reaction can be a living polymerization reaction, wherein a living polymer comprising a plurality of living end groups is formed. In various examples, a living polymerization reaction forms a living poly(cyclic acetal) comprising a plurality of living end groups independently chosen at each occurrence from halide-terminated end groups, triflate end groups, and the like, and any combination thereof.

In various examples, a method further comprising, after forming the poly(cyclic acetal), one or more or all of: adding one or more quenching agent(s) to the polymerization mixture; adding one or more base(s) to the polymerization mixture; removing residual acidic species in the polymerization mixture; or removing or isolating the poly(cyclic acetal) from the polymerization mixture. With the exception of the removing or isolating step, which must be the performed last, the additional steps can be performed in any order including simultaneously.

In various examples, quenching agent(s) is/are chosen from Lewis Base(s), Brønsted Base(s), and the like, and any combination thereof. In various examples, quenching agent(s) is/are sodium benzyloxide, or the like. In various examples, base(s) is/are chosen from alkali metal alkoxide(s), amine(s), and the like, and any combination thereof.

In various examples, a polymerization method for producing a poly(cyclic acetal) comprises combining: one or more cyclic acetal monomer(s); one or more organic cation salt catalyst(s); and one or more proton trap(s), to form a polymerization mixture, where a poly(cyclic acetal) is formed.

In various examples, cyclic acetal monomer(s) is/are cyclic methylene acetal monomer(s) or the like. In various examples, the cyclic methylene acetal monomer(s) is/are chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and the like, and any combination thereof.

In various examples, cyclic acetal monomer(s) is/are present in the polymerization mixture at from about 1.5 M to about 15 M, including all integer M values and ranges therebetween. In various examples, cyclic acetal monomer(s) is/are present in a polymerization mixture at about 1.5 M or greater, about 2 M or greater, about 3 M or greater, about 4 M or greater, about 5 M or greater, about 6 M or greater, about 7 M or greater, about 8 M or greater, about 9 M or greater, about 10 M or greater, about 11 M or greater, about 12 M or greater, about 13 mol or greater, about 14 mol or greater, or about 15 M. In various examples, cyclic acetal monomer(s) is/are present neat (e.g., in the absence of one or more solvent(s)).

In various examples, organic cation salt catalyst(s) is/are of the form C⁺A⁻, where a C⁺ group is chosen from electrophilic alkylating agents, and the like, and wherein an A⁻ group is chosen from non-nucleophilic anions, complex anions, non-complex anions, and the like, or the like. In various examples, a C⁺ group is chosen from carbenium groups, carboxonium groups, trityl groups, oxycarbenium groups, oxonium groups, and the like. In various examples, an A⁻ group is chosen from tetrafluoroborate (BF₄⁻), hexafluorophosphate (PF₆⁻), perchlorate (ClO₄⁻), triflate (SO₃CF₃⁻), hexafluoroantimonate, hexachloroantimonate, tetrakis[3,5-bis(trifluoromethyl)phenyl]borate (BArF₂₄⁻), tetrakis(pentafluorophenyl)borate (B(C₆F₅)₄⁻), perfluoroalkyl aluminates, and the like. In various examples, organic cation salt catalyst(s) is/are chosen from [(Et)₃O]BF₄, [(Et)₃O]PF₆, and the like, and any combination thereof.

In various examples, organic cation salt catalyst(s) is/are present in the polymerization mixture at 1×10⁻⁵ mM to 10 mM, including all integer mM values and ranges therebetween. In various examples, an initial molar ratio of the cyclic acetal monomer(s) to the organic cation salt catalyst(s) is from about 1000:1 to about 40,000:1, including all integer molar ratio values and ranges therebetween.

In various examples, proton trap(s) is/are sterically hindered base(s). In various examples, sterically hindered base(s) is/are chosen from 2,6-di-tert-butylpyridine, 2,6-di-tert-butyl-4-methylpyridine, 2,4,6-tri-tert-butylpyridine, 2,4,6-tri-tert-butylpyrimidine, and the like, and any combination thereof. In various examples, proton trap(s) is/are present in a polymerization mixture at from about 1×10⁻⁵ mM to about 10 mM, including all integer M values and ranges therebetween. In various examples, an initial molar ratio of cyclic acetal monomer(s) to proton trap(s) is from about 200:1 to about 40,000:1, including all integer molar ratio values and ranges therebetween.

In various examples, a polymerization mixture further comprises one or more chain transfer agent(s) (CTA(s)). In various examples, chain transfer agent(s) (CTA(s)) is/are acyclic acetal(s) chosen from diethoxymethane (DEM), dibutoxymethane (DBM), 1,1′-[Methylenebis(oxy)]bis[propane] (MOP), and the like, and any combination thereof. In various examples, chain transfer agent(s) (CTA(s)) is/are present in a polymerization mixture at from about 1×10⁻⁴ mM to about 6 mM including all integer mM values and ranges therebetween. In various examples, the initial molar ratio of the organic cation catalyst(s) to the CTA(s) is from about 1:1 to about 200:1, including all integer molar ratio values and ranges therebetween.

Various solvents can be used. In various examples, In various examples, the one or more solvent(s) is/are chosen from halocarbon solvents (e.g., dichloromethane, chloroform, or the like, or any combination thereof), aryl solvents (e.g., toluene, xylene, or the like), ether solvents, and the like, any combination thereof.

In various examples, a polymerization is carried out at a temperature of from about −90° C. to about 50° C., including all 0.1° C. values and ranges therebetween. The method of claim 36, wherein the temperature is from about 0° C. to about 25° C., including all 0.1° C. values and ranges therebetween.

In various examples, a monomer to polymer conversion is from about 20% to about 100% conversion, including all integer % values and ranges therebetween.

In various examples, a method forms a living poly(cyclic acetal) comprising a plurality of living end groups independently chosen at each occurrence from alkyl ether groups, aryl ether groups, alkylaryl ether groups, and the like, and any combination thereof.

In various examples, a method further comprises, after foiiiiing the poly(cyclic acetal), one or more or all of: adding one or more quenching agent(s) to the polymerization mixture; adding one or more base(s) to the polymerization mixture; removing residual acidic species in the polymerization mixture; or removing or isolating the poly(cyclic acetal) from the polymerization mixture. With the exception of the removing or isolating step, which must be the performed last, the additional steps can be performed in any order including simultaneously.

In various examples, quenching agent(s) is/are chosen from Lewis Base(s), Bronsted Base(s), or the like, or any combination thereof. In various examples, quenching agent(s) is/are sodium benzyloxide, or the like. In various examples, base(s) is/are chosen from alkali metal alkoxide(s), amine(s), or any combination thereof.

In an aspect, the present disclosure provides methods of depolymerizing one or more poly(cyclic acetal(s)). Non-limiting examples of methods of depolymerizing poly(cyclic acetal(s)) are provided herein (e.g., in the Statements and the Examples).

In various examples, a depolymerization process comprises: combining one or more poly(cyclic acetal(s)) and one or more acid catalyst(s) to form a depolymerization mixture; and heating the depolymerization mixture to form said cyclic acetal(s). In various examples, one or more poly(cyclic acetal(s)) are comprised in one or more composition(s). In various examples, the depolymerization mixture does not comprise any glycol compounds.

In various examples, the one or more acid catalyst(s) comprise strong acids (e.g., acids having a pKₐ of <less than or equal to 4). In various examples, the acid catalyst(s) is/are mineral acids. In various examples, acid catalyst(s) is/are chosen from camphorsulfonic acid (CSA), diphenylphosphoric acid (DPA), sulfonic acid(s) (SA(s)), phosphoric acid(s) (PA(s)), and the like, and any combination thereof. In various examples, acid catalyst(s) comprise(s) a solid support. In various examples, acid catalyst(s) are polymeric acid catalyst(s). In various examples, polymeric acid catalyst(s) is/are chosen from poly(styrene sulfonic acid), or the like. In various examples, acid catalyst(s) is/are chosen from poly(styrene sulfonic acid) resin(s). In various examples, a depolymerization mixture comprises greater than or equal to 0.01 mol % or greater of acid based on the total moles of poly(cyclic acetal(s)) and acid catalyst(s).

In various examples, a depolymerization mixture is heated at a pressure of from about 1×10⁻⁴ atm to about 5.0 atm, including all 1×10⁻⁵ atm values and ranges therebetween. In various examples, the depolymerization mixture is heated to a temperature of from about 25° C. to about 300° C., including all 0.1° C. values and ranges therebetween.

In various examples, a method further comprises, after forming said cyclic acetal(s), removing or isolating said cyclic acetal(s) from the depolymerization mixture. In various examples, removing or isolating said cyclic acetal(s) is performed via distillation of said cyclic acetal(s) from the depolymerization mixture.

The following Statements describe various examples of methods, products and systems of the present disclosure and are not intended to be in any way limiting:

Statement 1. A poly(cyclic acetal) comprising: a number-average molecular weight (Mₙ) of from about 10 kiloDaltons (kDa) to about 3000 kDa, and where greater than about 50% of the poly(cyclic acetal) chain ends are not hydroxyl groups.
Statement 2. A poly(cyclic acetal) according to Statement 1, where the number-average molecular weight (Mₙ) is from about 100 kiloDaltons (kDa) to about 1000 kDa, including all integer kDa values and ranges therebetween.
Statement 3. A poly(cyclic acetal) according to Statement 1 or 2, where the chain ends are independently chosen at each occurrence from alkyl ether groups, aryl ether groups, alkyl aryl ether groups, heterocyclic groups, halide groups, triflate groups, amine groups, thiol groups, or phosphine groups, or the like.
Statement 4. A poly(cyclic acetal) according to any one of the preceding Statements, where the poly(cyclic acetal) comprises one or more homopolymer(s) or copolymer(s) of poly(1,3-dioxepane) (PDXP), poly(1,3-dioxocane) (PDXC), poly(1,3,6-trioxocane) (PTXC), poly(1,3-hexahydrobenzodioxole) (PHBD), poly(1,3-dioxolane) (PDXL), or the like, or any combination thereof.
Statement 5. A poly(cyclic acetal) according to any one of the preceding Statements, where the poly(cyclic acetal) comprises one or more or all of the following: a thermal stability (T_{d,5%}) of from about 337° C. to about 392° C., including all 0.1° C. values and ranges therebetween; a thermal stability (T_{d,50%}) of from about 377° C. to about 462° C., including all 0.1° C. values and ranges therebetween, with or without 2 mol % of one or more additive(s) having a pKₐ greater than 4; an Arrhenius activation energy (Eₐ) of about 85.0 kJ/mol, including all 0.1 kJ/mol values and ranges therebetween, with 2 mol % of one or more acid(s) having a pKₐ less than or equal to 4; a melting temperature (Tₘ ) of from about 0° C. to about 120° C., including all 0.1° C. values and ranges therebetween; a glass transition temperature (T_{g}) of from about −100° C. to about 100° C., including all 0.1° C. values and ranges therebetween; a tensile stress at break about 10 MPa to about 50 MPa, including all 0.1 MPa values and ranges therebetween; or a tensile strain at break of from about 1% to about 800%, including all 0.1% values and ranges therebetween.
Statement 6. A poly(cyclic acetal) according to any one of the preceding Statements, where the poly(cyclic acetal) is in the form of a solution, an emulsion, a slurry, a dispersion, a particle, a flake, a pellet, a powder, a granule, a tube, a sphere, a fiber, a foam, a film, a textile, a mesh, a sheet, a bar, a monolith, or the like.
Statement 7. An article of manufacture comprising one or more poly(cyclic acetal)(s) according to any one of the preceding Statements.
Statement 8. An article of manufacture according to Statement 7, where the article is a spun article, a molded article, an extruded article, a coated article, a blown article, a woven article, a drawn article, a laminated article, a 3D printed article, or the like.
Statement 9. A method of making a poly(cyclic acetal), the method comprising: combining one or more cyclic acetal monomer(s); one or more haloalkyl ether initiator(s); and one or more Lewis acid catalyst(s), to form a polymerization mixture, where a poly(cyclic acetal) is formed.
Statement 10. A method according to Statement 9, where the cyclic acetal monomer(s) is/are cyclic methylene acetal monomer(s) chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and the like and any combination thereof.
Statement 11. A method according to Statement 9 or 10, where the cyclic acetal monomer(s) is/are present in the polymerization mixture at from about 1.5 M to about 15 M, including all 0.05 M values and ranges therebetween, or neat.
Statement 12. A method according to any one of Statements 9 to 11, where the haloalkyl ether initiator(s) comprise(s) a C₁-C₂₅ haloalkyl group chosen from linear, cyclic, branched, substituted alkyl, unsaturated, and saturated C₁-C₂₅ haloalkyl group(s), and the like, and any combination thereof.
Statement 13. A method according to Statements 9 to 12, where the haloalkyl ether initiator(s) is/are chosen from benzyl chloromethyl ether, bromomethyl methyl ether, 1-chloromethyl adamantane, chloromethyl cyclohexyl ether, chloromethyl dococosyl ether, chloromethyl ethyl ether, chloromethyl methyl ether, tert-butyl chloromethyl ether, methoxyethyl chloromethyl ether, and the like, and any combination thereof.
Statement 14. A method according to Statements 9 to 13, where: the haloalkyl ether initiator(s) is/are present in the polymerization mixture at 1×10⁻⁶ mM to 1 mM, including all 0.0000005 mM values and ranges therebetween;; the initial molar ratio of the cyclic acetal monomer(s) to the haloalkyl ether initiator(s) is from about 200:1 to about 1600:1, including all integer initial molar ratio values and ranges therebetween; or both; or both.
Statement 15. A method according to Statements 9 to 14, where the Lewis acid catalyst(s) is/are Lewis acid(s) of the form MXₙ, where M=Ga, In, Zn, Sb^{V}, Sn^{IV}, Fe, or the like, and where X=Cl, Br, I, OTf, or the like.
Statement 16. A method according to Statements 9 to 15, where: the Lewis acid catalyst(s) is/are present in the polymerization mixture at 1×10⁻⁵ mM to 2 mM, including all 0.000005 mM values and ranges therebetween; the initial molar ratio of the cyclic acetal monomer(s) to the Lewis acid catalyst(s) is from about 200:1 to about 1600:1, including all integer initial molar ratio values and ranges therebetween; or both.
Statement 17. A method according to Statements 9 to 16, where the polymerization mixture further comprises one or more proton trap(s).
Statement 18. A method according to Statement 17, where the proton trap(s) is/are sterically hindered base(s) chosen from 2,6-di-tert-butylpyridine (DTPB), 2,6-di-tert-butyl-4-methylpyridine, 2,4,6-tri-tert-butylpyridine, 2,4,6-tri-tert-butylpyrimidine, and the like, and any combination thereof.
Statement 19. A method according to Statement 17 or 18, where: the proton trap(s) is/are present in the polymerization mixture at from about 2×10⁻⁵ mM to about 1 mM, including all 0.000001 mM values and ranges therebetween; the initial molar ratio of the cyclic acetal monomer(s) to the proton trap(s) is from about 200:1 to about 1600:1 including all integer initial molar ratio values and ranges therebetween; or both.
Statement 20. A method according to Statements 9 to 19, where the polymerization is carried out at a temperature of from about −90° C. to about 50° C., including all 0.1° C. values and ranges therebetween.
Statement 21. A method according to Statements 9 to 20, where the monomer to polymer conversion is from about 20% to about 100% conversion, including all integer % conversion values and ranges therebetween.
Statement 22. A method according to Statements 9 to 21, the method further comprising, after forming the poly(cyclic acetal), one or more or all of the following: adding one or more quenching agent(s) to the polymerization mixture; adding one or more base(s) to the polymerization mixture; removing residual acidic species in the polymerization mixture; or removing or isolating the poly(cyclic acetal) from the polymerization mixture.
Statement 23. A polymerization method for making a poly(cyclic acetal), the method comprising: combining one or more cyclic acetal monomer(s); one or more organic cation salt catalyst(s); and one or more proton trap(s), to form a polymerization mixture, where a poly(cyclic acetal) is formed.
Statement 24. A method according to Statement 23, where the cyclic acetal monomer(s) is/are cyclic methylene acetal monomer(s) chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and the like, and any combination thereof.
Statement 25. A method according to Statement 23 or 24, where the cyclic acetal monomer(s) is/are present in the polymerization mixture at from about 1.5 M to about 15M, including all 0.05 M values and ranges therebetween, or neat.
Statement 26. A method according to any one of Statements 23 to 25, where the organic cation salt catalyst(s) is/are of the form C⁺A⁻, where C⁺ is chosen from electrophilic alkylating agents, and where A⁻ is chosen from non-nucleophilic anions, complex anions, non-complex anions, and the like, and any combination thereof.
Statement 27. A method according to Statement 26, where C⁺=carbenium, carboxonium, trityl, oxycarbenium, oxonium, or the like, and A⁻=tetrafluoroborate (BF₄⁻), hexafluorophosphate (PF₆⁻), triflate (SO₃CF₃⁻), perchlorate (ClO₄⁻), hexafluoroantimonate, hexachloroantimonate, perfluoroalkyl aluminates, tetrakis[3,5-bis(trifluoromethyl)phenyl]borate (BArF₂₄⁻), tetrakis(pentafluorophenyl)borate (B(C₆F₅)₄⁻), or the like.
Statement 28. A method according to Statement 27 or 28, where the organic cation salt catalyst(s) is/are chosen from [(Et)₃O]BF₄, [(Et)₃O]PF₆, and or the like, and any combination thereof.

Statement 29. A method according to any one of Statements 23 to 28, where: the organic cation salt catalyst(s) is/are present in the polymerization mixture at 1×10⁻⁵ mM to 10 mM, including all 0.000005 mM values and ranges therebetween; the initial molar ratio of the cyclic acetal monomer(s) to the organic cation salt catalyst(s) is from about 1000:1 to about 40,000:1 including all integer initial molar ratio values and ranges therebetween; or both.

Statement 30. A method according to any one of Statements 23 to 29, where the proton trap(s) is/are sterically hindered base(s) chosen from 2,6-di-tert-butylpyridine, 2,6-di-tert-butyl-4-methylpyridine, 2,4,6-tri-tert-butylpyridine, 2,4,6-tri-tert-butylpyrimidine, and the like, and any combination thereof.
Statement 31. A method according to Statement 30, where: the proton trap(s) is/are present in the polymerization mixture at from about 1×10⁻⁵ mM to about 10 mM, including all 0.000005 mM values and ranges therebetween; the initial molar ratio of the cyclic acetal monomer(s) to the proton trap(s) is from about 200:1 to about 40,000:1, including all integer molar ratio values and ranges therebetween; or both.
Statement 32. A method according to any one of Statements 23 to 231, where the polymerization mixture further comprises one or more chain transfer agent(s) (CTA(s)).
Statement 33. A method according to Statement 32, where the chain transfer agent (CTA(s)) is/are acyclic acetal(s), or the like.
Statement 34. A method according to Statement 32 or 33, where: the chain transfer agent(s) (CTA(s)) is/are present in the polymerization mixture at from about 1×10⁻⁴ mM to about 6 mM, including all 0.00005 mM values and ranges therebetween; the initial molar ratio of the organic cation catalyst(s) to the CTA(s) is from about 1:1 to about 200:1, including all integer molar ratio values and ranges therebetween; or both.
Statement 35. A method according to any one of Statements 23 to 34, where the polymerization is carried out at a temperature of from about −90° C. to about 50° C., including all 0.1° C. values and ranges therebetween.
Statement 36. A method according to any one of Statements 23 to 35, where the monomer to polymer conversion is from about 20% to about 100% conversion, including all integer % conversion values and ranges therebetween.
Statement 37. A method according to any one of Statements 23 to 36, the method further comprising, after forming the poly(cyclic acetal), one or more or all of: adding one or more quenching agent(s) to the polymerization mixture; adding one or more base(s) to the polymerization mixture; removing residual acidic species in the polymerization mixture; or removing or isolating the poly(cyclic acetal) from the polymerization mixture.
Statement 38. A depolymerization method comprising: combining: one or more poly(cyclic acetal(s)); and one or more acid catalyst(s) having a pKₐ of less than or equal to 4, to form a depolymerization mixture; and heating the depolymerization mixture at a pressure of from about 1×10⁻⁴ atm to about 5.0 atm, including all 0.00001 atm values and ranges therebetween, to form said cyclic acetal(s).
Statement 39. A depolymerization method according to Statement 38, where the acid catalyst(s) is/are chosen from camphorsulfonic acid (CSA), diphenylphosphoric acid (DPA), sulfonic acid(s) (SA(S)), phosphoric acid(s) (PA(s)), and the like, and any combination thereof.
Statement 40. A depolymerization method according to Statement 38 or 39, where the acid catalyst(s) comprise(s) a solid support.
Statement 41. A depolymerization method according to any one of Statements 38 to 40, where the depolymerization mixture comprises greater than or equal to 0.01 mol % of acid or greater, based on the total moles of poly(cyclic acetal) and acid catalyst(s).
Statement 42. The depolymerization method according to any one of Statements 38 to 41, where the depolymerization mixture is heated to a temperature of from about 25° C. to about 300° C., including all 0.1° C. values and ranges therebetween.
Statement 43. The depolymerization method according to any one of Statements 38 to 42, further comprising removing or isolating said cyclic acetal(s) from the depolymerization mixture.

The steps of the methods described in the various examples disclosed herein are sufficient to carry out a method of the present disclosure. Thus, in various examples, a method consists essentially of a combination of the steps of the methods disclosed herein. In various other examples, a method consists of such steps.

The following examples are presented to illustrate the present disclosure. They are not intended to be limiting in any matter.

### EXAMPLE 1

The following is an example of chemically recyclable polyacetals with polyolefin-like properties, methods of making same, and methods of using same.

Living cationic polymerizations were first introduced in the 1970's by Higashimura and Sawamoto who used I₂ and I₂/HI initiators to polymerize vinyl ethers and N-vinylcarbazole. Kennedy and Faust later reported on the living cationic polymerization of isobutylene using a cumyl acetate/BCl₃ initiator system. Recently, Aoshima and coworkers copolymerized vinyl ethers and cyclic acetals by SnCl₄/TiCl₄-catalyzed cationic polymerization using initiators derived from vinyl ether-hydrochloride adducts. In these systems, a low cation concentration is required to suppress termination and chain-transfer side reactions and is achieved by incorporating dormant halide-terminated chain-ends, which can undergo reversible deactivation in the presence of a corresponding Lewis acid catalyst Living cationic polymerizations were first introduced in the 1970's by Higashimura and Sawamoto who used I₂ and I₂/HI initiators to polymerize vinyl ethers and N-vinylcarbazole. Kennedy and Faust later reported on the living cationic polymerization of isobutylene using a cumyl acetate/BCl₃ initiator system. Recently, Aoshima and coworkers copolymerized vinyl ethers and cyclic acetals by SnCl₄/TiCl₄-catalyzed cationic polymerization using initiators derived from vinyl ether-hydrochloride adducts. In these systems, a low cation concentration is required to suppress termination and chain-transfer side reactions and is achieved by incorporating dormant halide-terminated chain-ends, which can undergo reversible deactivation in the presence of a corresponding Lewis acid catalyst.

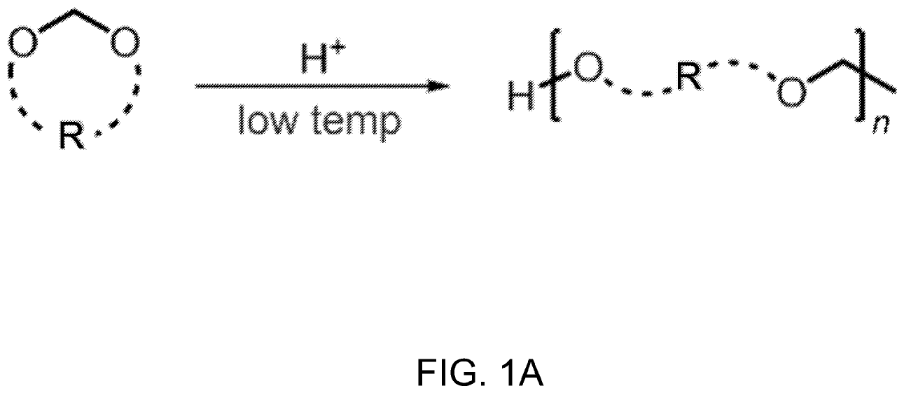

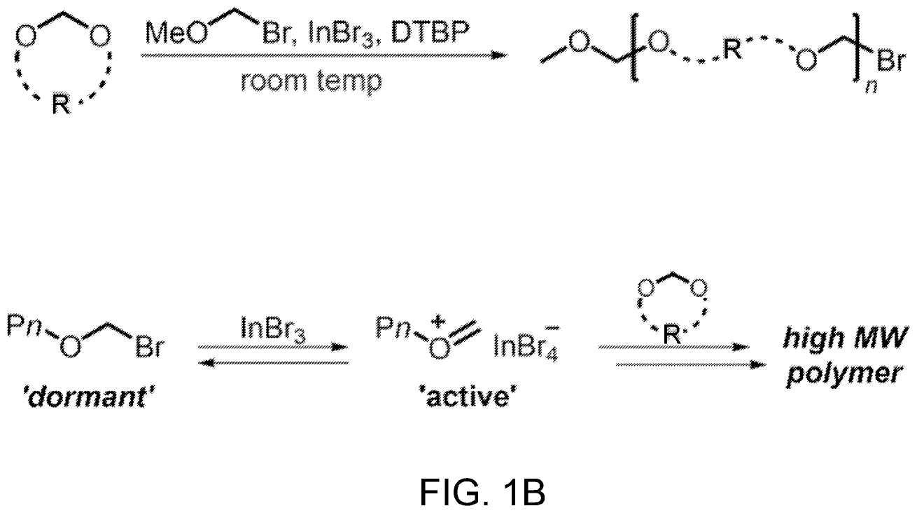

Identifying and synthesizing mechanically useful plastics capable of chemical recycling to monomer (CRM) are the foremost challenges in creating a circular plastic economy. Cyclic methylene acetals (CAs) are a diverse class of monomers that can be easily synthesized from diols and paraformaldehyde; however, uncontrolled cationic ring-opening polymerization (CROP) of CAs has precluded reliable access to high molecular weight polyacetals with useful mechanical properties. Here, the controlled reversible-deactivation CROP of CAs is presented using a commercially available InBr₃ catalyst and halomethyl ether initiator (FIG. 1B). Using this method, poly(1,3-dioxolane) (PDXL) was synthesized a tough thermoplastic with comparable mechanical properties to several commodity polyolefins and excellent thermal and chemical stability. The strong-acid-catalyzed depolymerization of PDXL and collection of pure monomer in near quantitative yields via distillation at moderate temperature were demonstrated, even from a mixture of commodity plastic waste. PDXL is a strong candidate for a commercial plastic capable of CRM, as abundant monomer feedstocks can be efficiently polymerized to afford a tough, thermally stable thermoplastic that can undergo triggered depolymerization for monomer feedstock recovery.

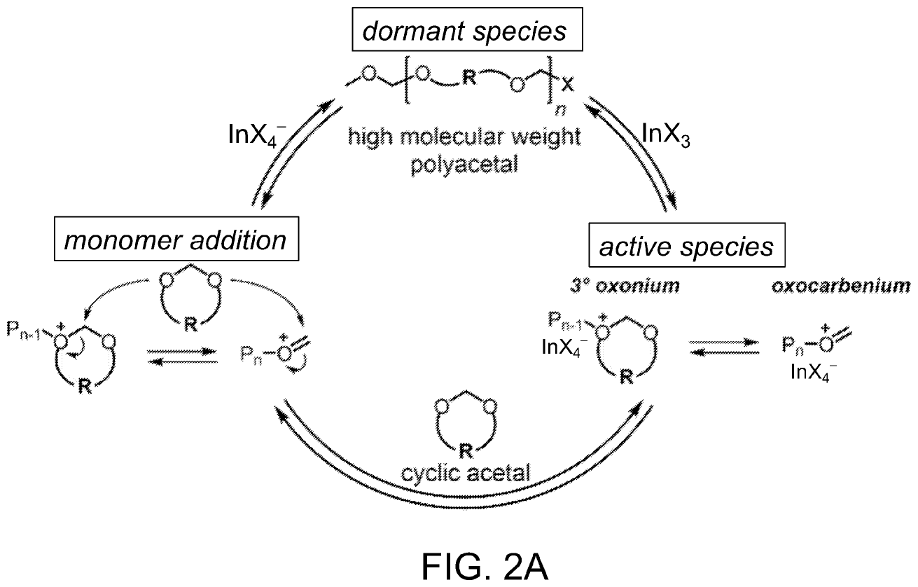

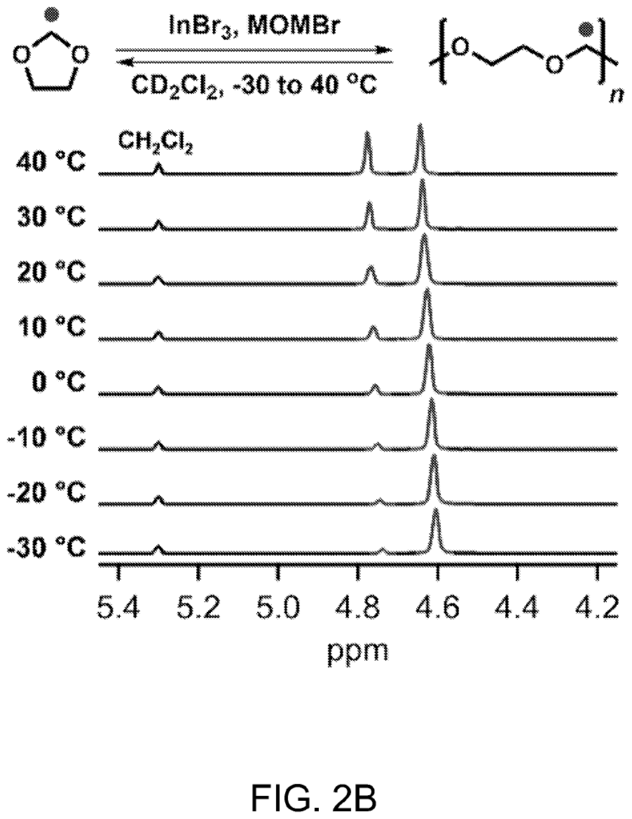

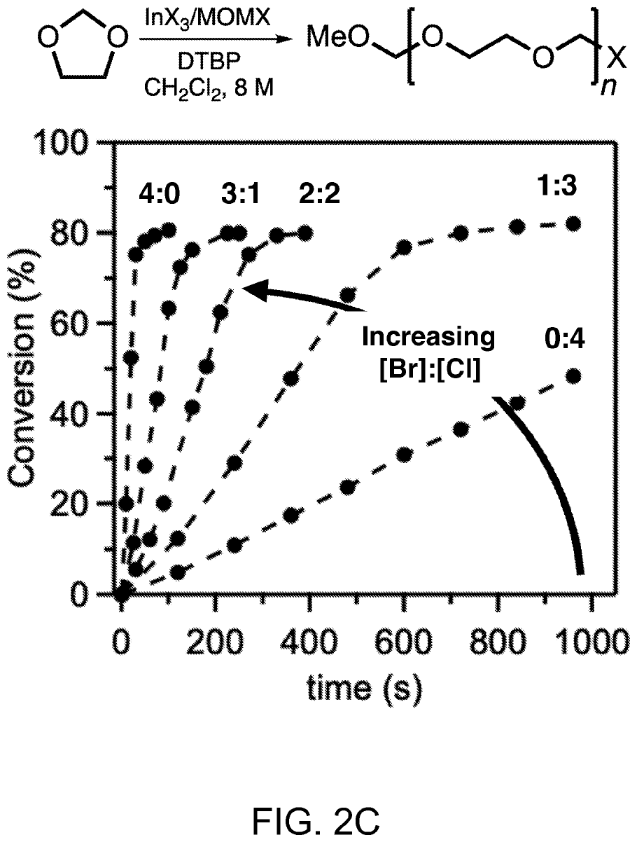

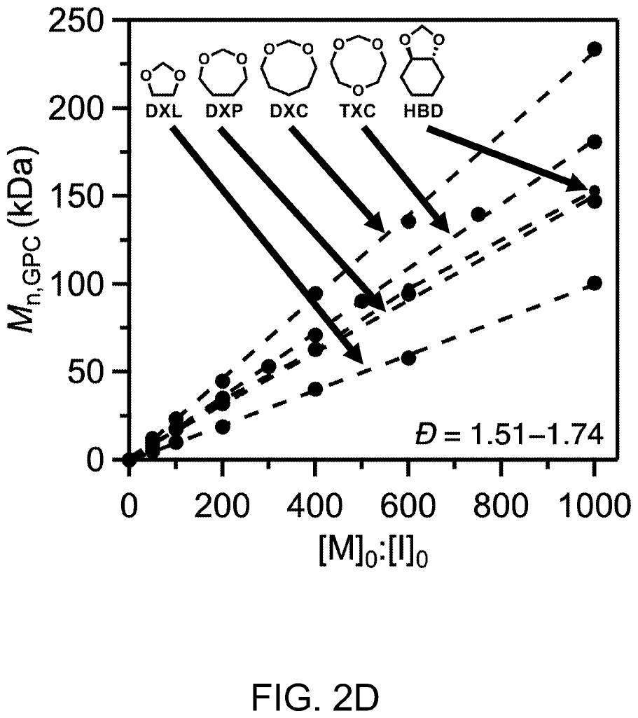

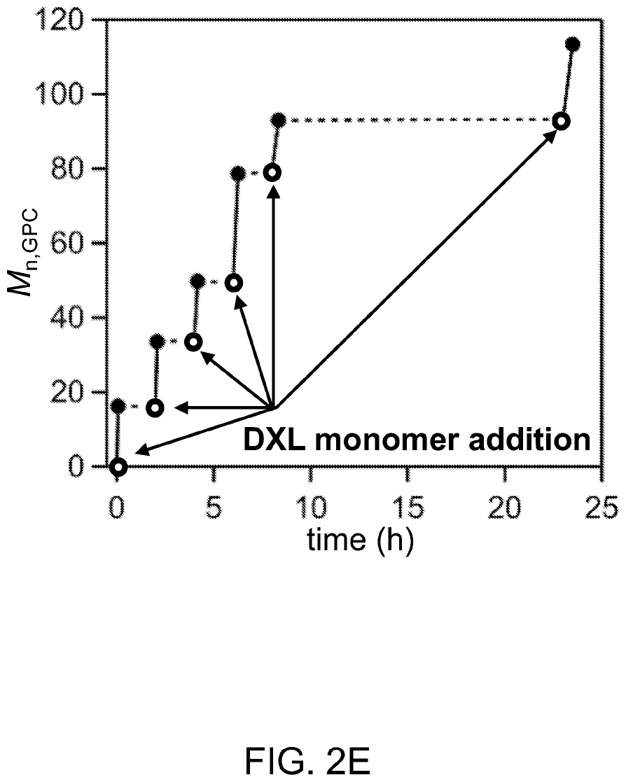

Inspired by the reversible-deactivation methods used to impart molecular weight control in the cationic polymerizations of olefinic monomers, a living reversible-deactivation CROP (RD-CROP) of cyclic acetals was sought to reliably obtain polyacetals of suitably high molecular weights to achieve useful mechanical properties. It was reasoned that employing a discrete initiator could be used in conjunction with an appropriate Lewis acid would enable reversible-deactivation polymerization behavior. Halomethyl ether initiators were chosen for this work due to their chemical similarity to the dormant polymer chain ends anticipated to form during RD-CROP of cyclic acetals when using halide-based initiators (FIG. 2A). To this end, chloromethyl methyl ether (MOMCl) was employed as the initiator for initial Lewis acid catalyst screening experiments.

A range of commercially available Lewis acids of the form MClₙ, where M=B, Al, Ga, In, Sb^{III}/Sb^{V}, Bi, Sn, Zn, Fe, Ti, Zr, or Hf were selected for catalyst screening experiments based upon their reported uses as Lewis acid catalysts for cationic polymerizations and/or Friedel-Crafts reactions. The selectivity of each Lewis acid towards controlled RD-CROP of DXL was evaluated by performing reactions using DXL monomer ([DXL]₀=8 M), MClₙ (5 mM or 10 mM), 2,6-di-tert-butyl pyridine (DTBP, 5 mM) and either 0 or 1 equiv of MOMCl (0 or 27 mM, [DXL]₀:[MOMCl]₀=300:1) in CH₂Cl₂ at room temperature in a glove box. Sterically hindered bases such as DTBP are employed as ‘proton traps’ in cationic polymerizations to prevent undesired initiation or chain transfer by protic impurities. Catalysts were deemed viable for controlled RD-CROP if polymerization only occurred when both initiator and Lewis acid catalyst were present during the reaction. No polymerization was observed within 18 h when Lewis acids based upon B, Al, Sb^{III}, Bi, Ti, Zr, and Hf were employed in the presence or absence of MOMCl (Table 2). Meanwhile, polymerization occurred in both the presence and absence of MOMCl when Ga, Sb^{V}, Sn, and Fe-based Lewis acids were employed (Table 2) indicating that these Lewis acids directly initiate DXL polymerization.

Notably, InCl₃ and ZnCl₂ selectively catalyzed the polymerization of DXL exclusively in the presence of MOMCl, achieving 84% and 49% conversion, respectively in 18 h at [MClₙ]=5 mM (Table 2). Increasing the Lewis acid concentration to 10 mM had no influence on DXL conversion after 18 h when InCl₃ was employed as the catalyst (84% cony), whereas monomer conversion increased to 78% in 18 h for the ZnCl₂-catalyzed polymerization (Table 2). It was reasoned that under these reaction conditions ([DXL]₀=8 M, T_{r×n}=22° C.), the polymerization of DXL reaches equilibrium at ˜85% conversion ([DXL]_{eq}=1.2 M) as anticipated based upon the measured ΔH° and ΔS° values for DXL polymerization. Thus, the InCl₃-catalyzed polymerization of DXL reached maximum conversion in less than 18 h, even at lower loadings (5 mM), and was chosen for further optimization based upon its higher activity towards RD-CROP of DXL as compared to ZnCl₂.

The rate of propagation in reversible-deactivation cationic polymerizations depends in part upon the extent of chain-end ionization by the Lewis acid catalyst. Improving the leaving group ability of the halide on the dormant chain end shifts the dormant-active equilibrium towards the active cationic species, increasing the rate of propagation. It was anticipated that by exchanging the Cl substituents of InCl₃/MOMCl for Br substituents, the polymerization rate could be increased. Accordingly, a series of RD-CROP kinetic experiments were performed in CH₂Cl₂ at 22° C. ([DXL]₀=8 M, [DTBP]₀=25 mM, InX₃=25 mM, MOMX=25 mM) and the halide composition ([Br]:[Cl]) systematically varied from 0:4 to 4:0 by combining the appropriate amounts of MOMCl, MOMBr, InCl₃, and InBr₃, to achieve a given halogen stoichiometry. As shown in FIG. 2C, polymerization rate increases with increasing [Br]:[Cl], with DXL conversion reaching 81% in 1.5 min when [Br]:[Cl]=4:0, as compared to 81% conversion in 40 min when [Br]:[Cl]=0:4 (Table 3). Controlled polymerization behavior was also observed for each [Br]:[Cl] stoichiometry as evidenced by the linear dependence of M_{n,GPC} on DXL conversion (Table 3).

As shown in FIG. 2D, molecular weight control during RD-CROP of cyclic acetals using the InBr₃/MOMBr catalyst system was achieved for a range of monocyclic acetal derivatives including DXL, 1,3-dioxepane (DXP), 1,3-dioxecane (DXC), 1,3,6-trioxecane (TXC), and bicyclic acetal trans-hexahydro-1,3-benzodioxole (HBD). For each cyclic acetal derivative, M_{n,GPC} increased linearly with increasing [monomer]₀:[MOMBr]₀ which is indicative of exclusive initiation by MOMBr. Of note are the high Ð values ranging from 1.51-1.74 for the M_{n,GPC} values reported in FIG. 2B. High Ð values during polymerizations exhibiting molecular weight control can result from slow initiation relative to propagation, when the rate of depropagation is equal to or near the rate of propagation, or chain transfer to polymer. The rates of initiation and propagation were anticipated to be similar due to the common bromomethyl ether functional groups present in both MOMBr and the polymer chain ends. Elevated Ð values (>1.4) were also observed at low DXL conversions (Table 3, entries 1a-i) when the rate of depropagation is equal to or near the rate of propagation indicating that the equilibrium nature of RD-CROP of cyclic acetals is not exclusively responsible for the high Ð values.

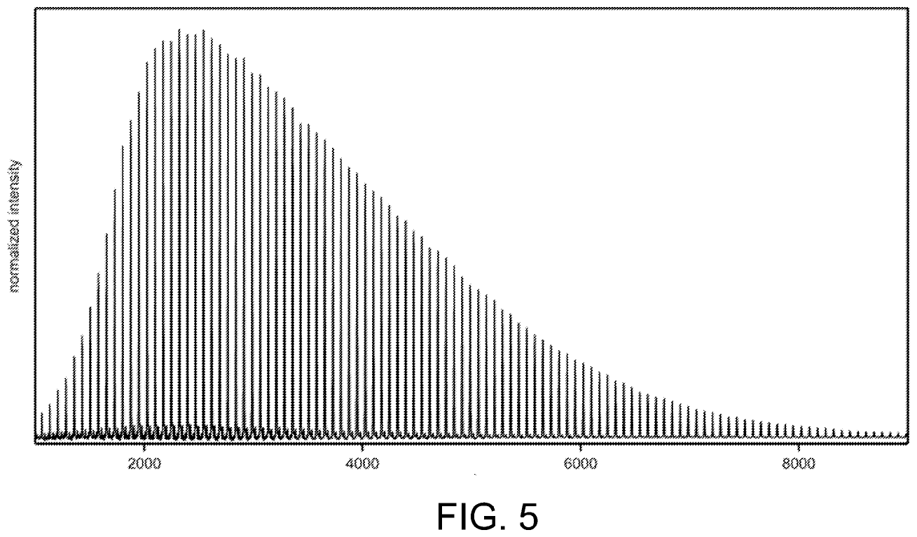

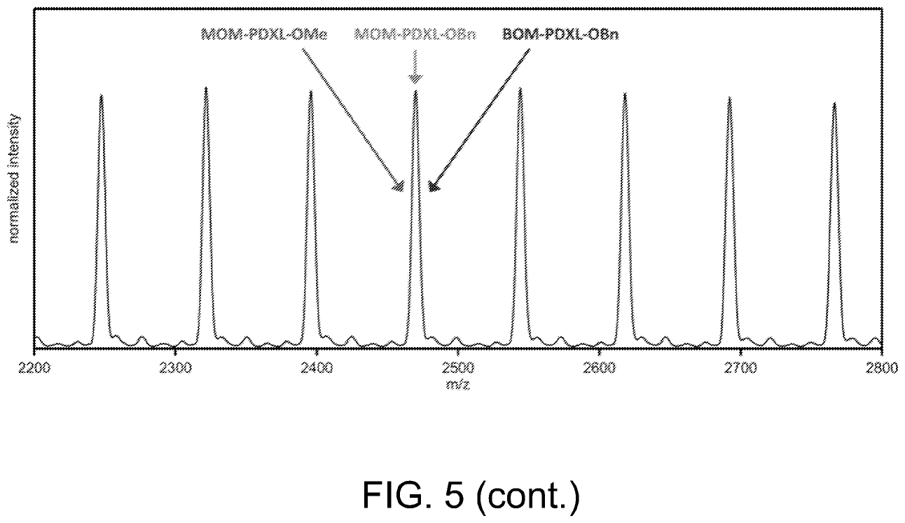

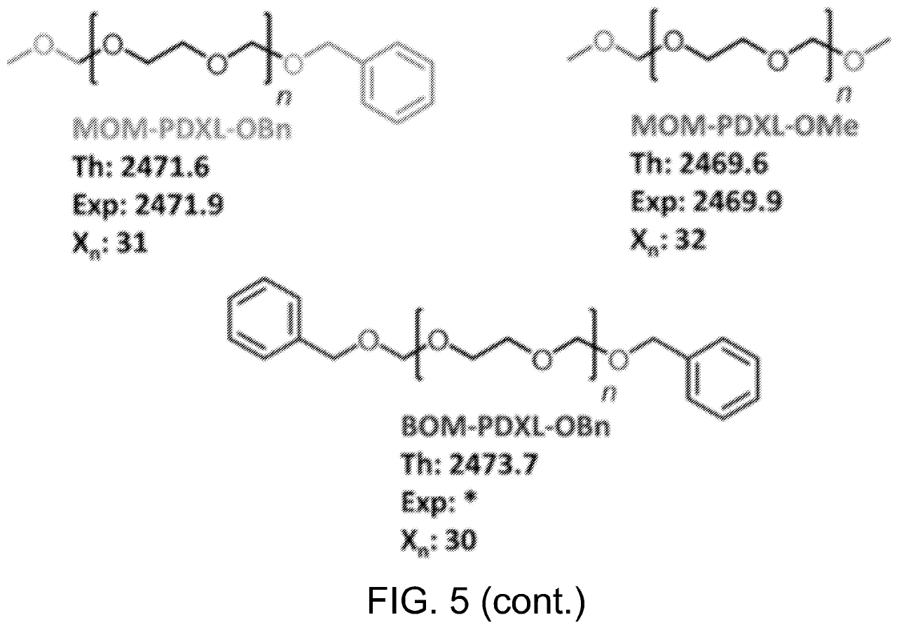

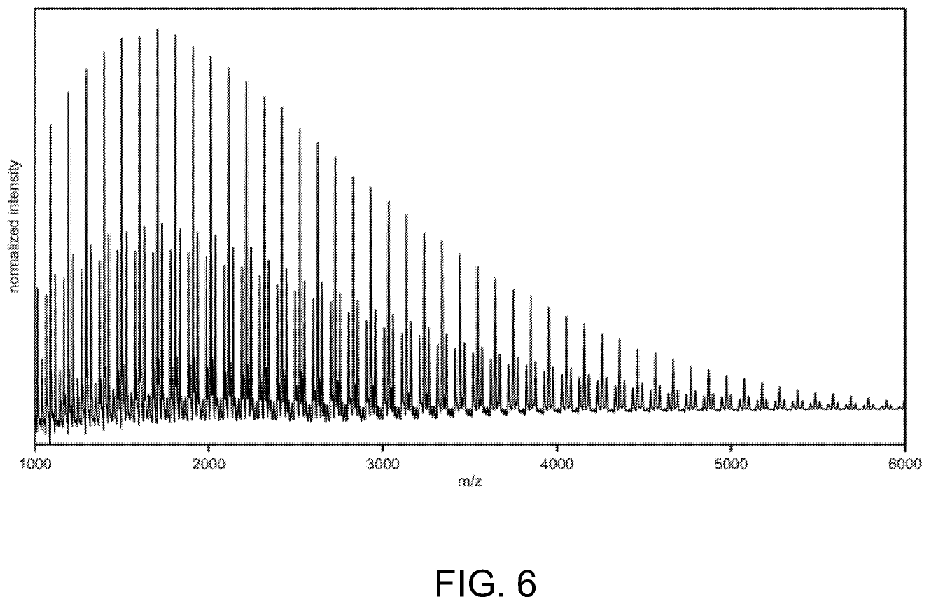

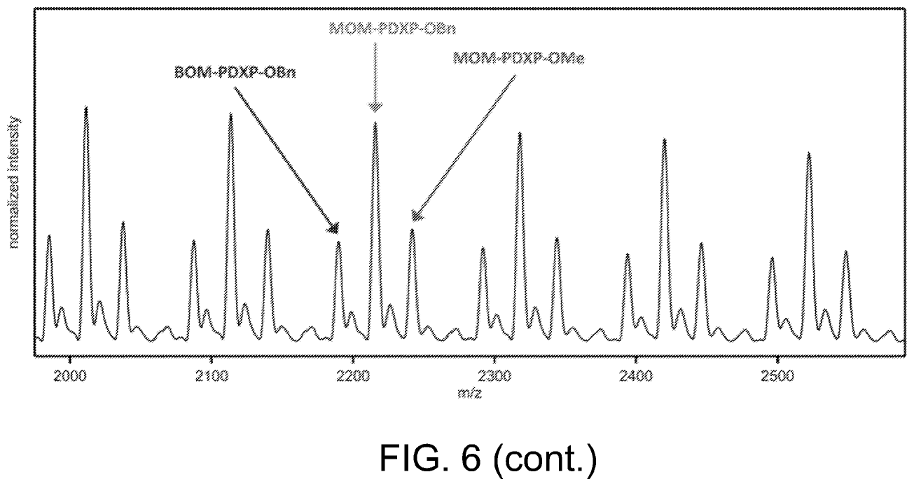

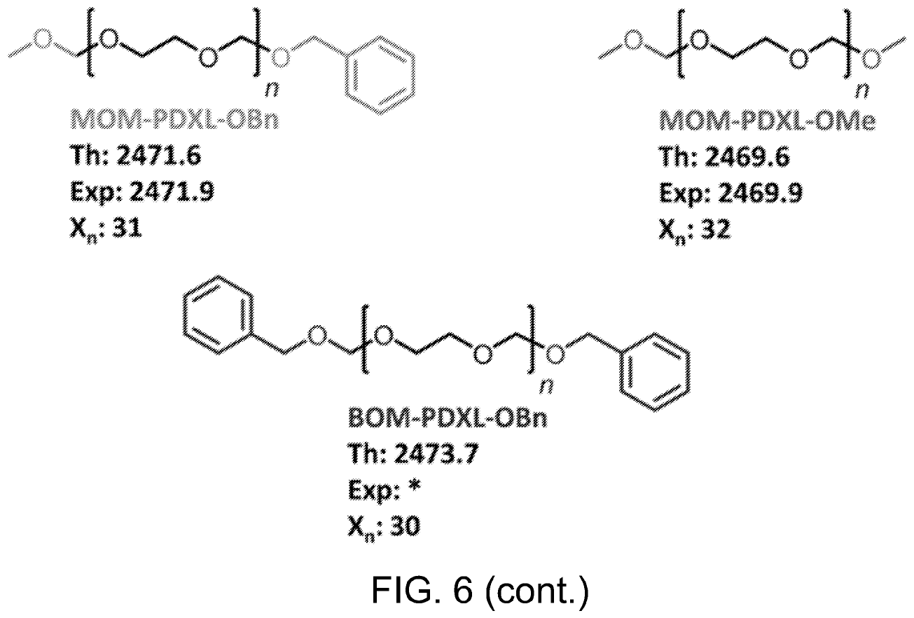

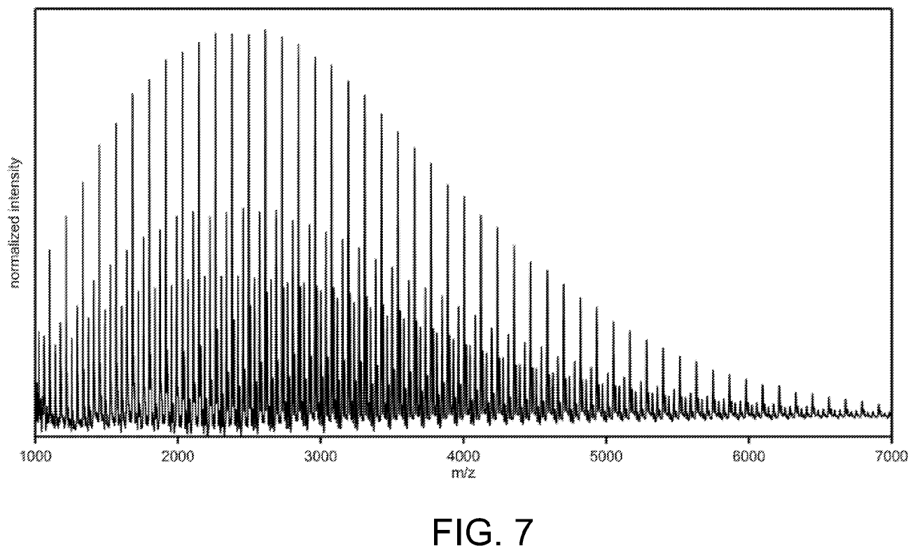

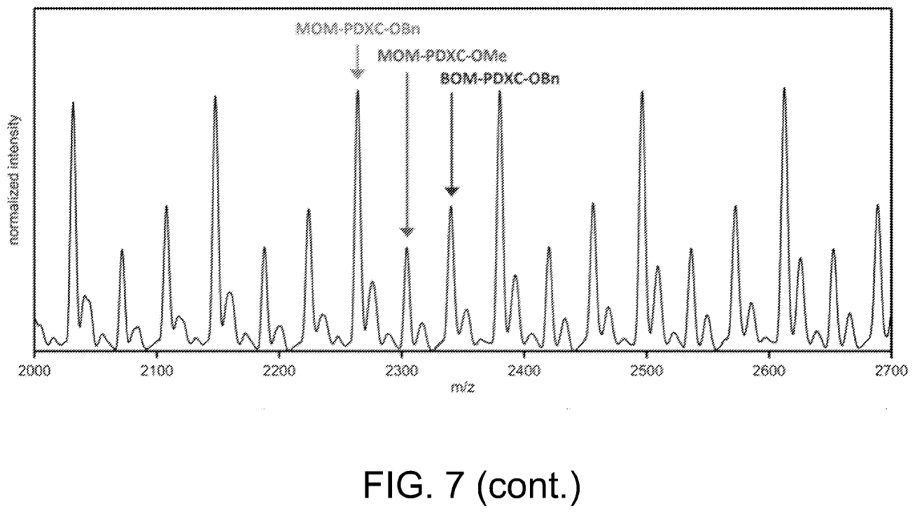

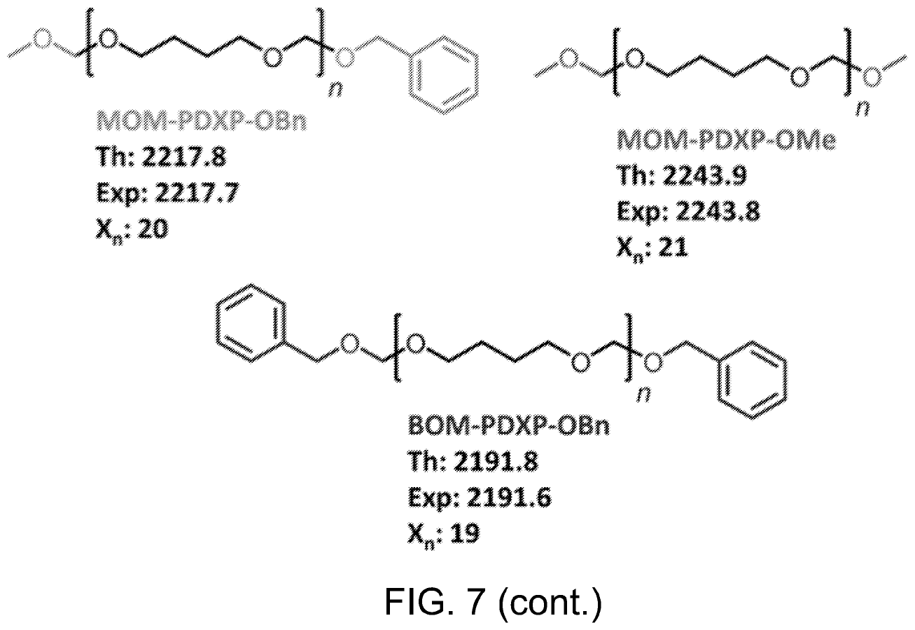

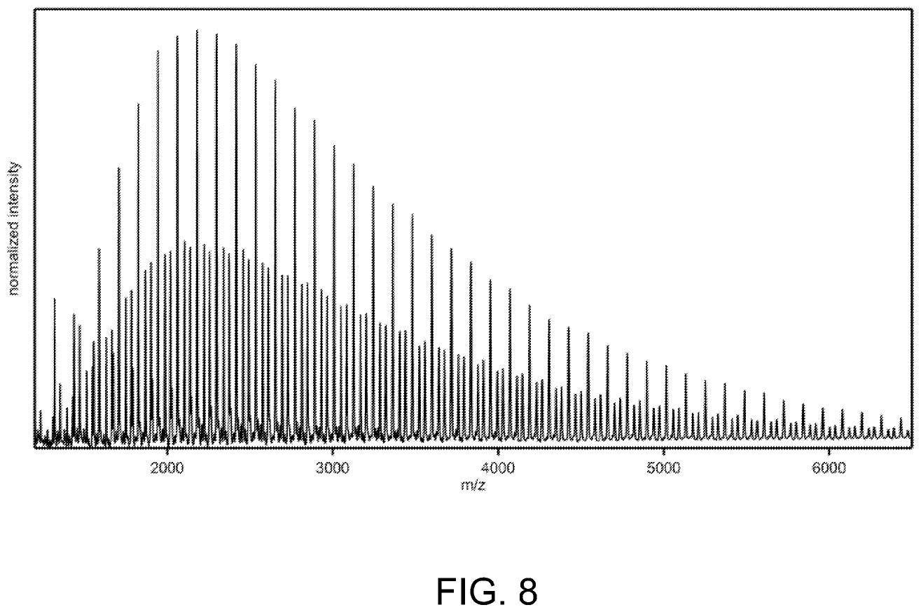

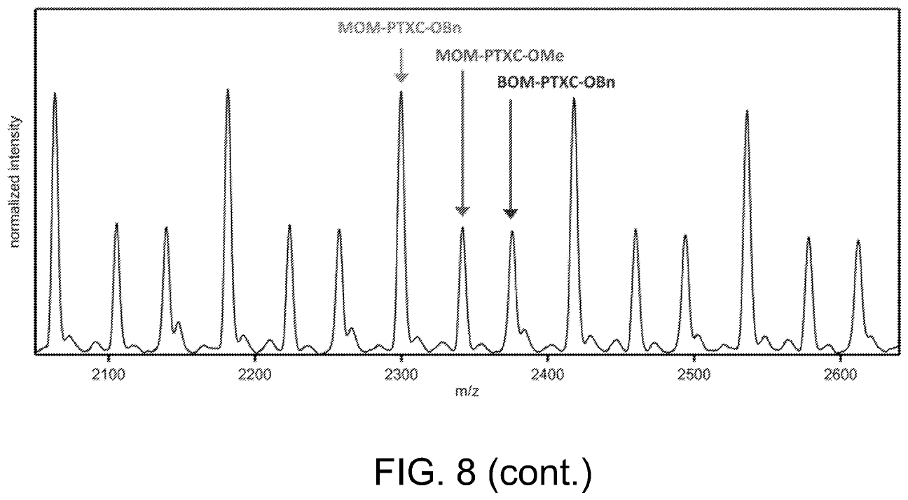

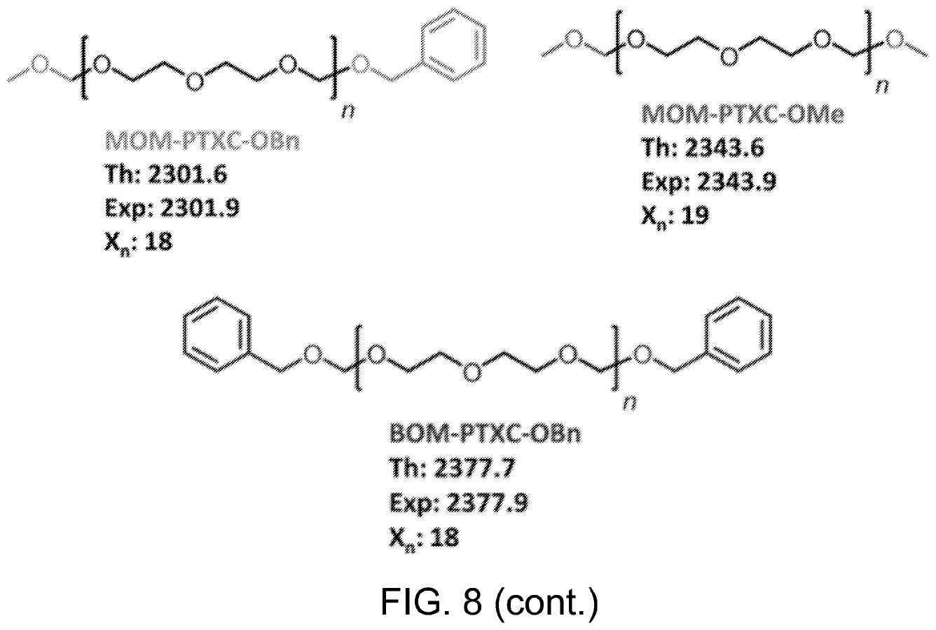

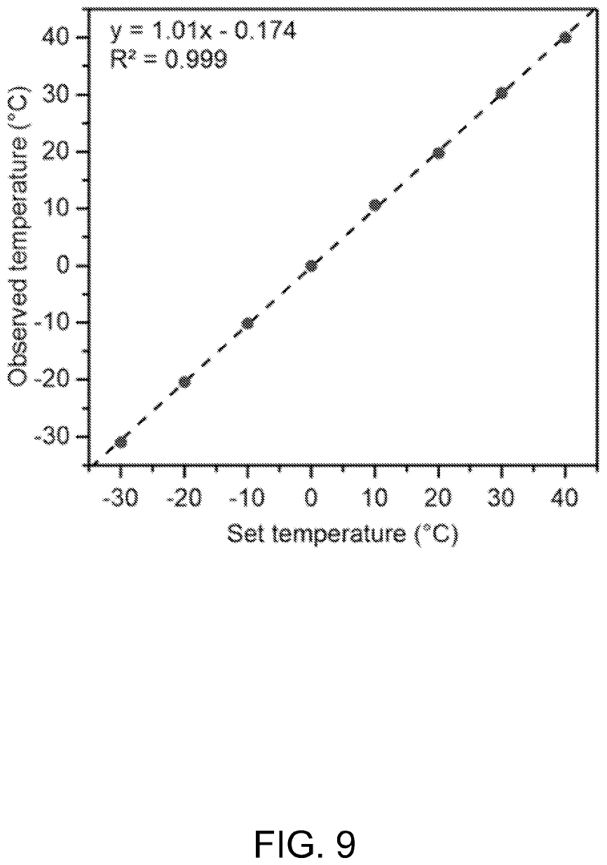

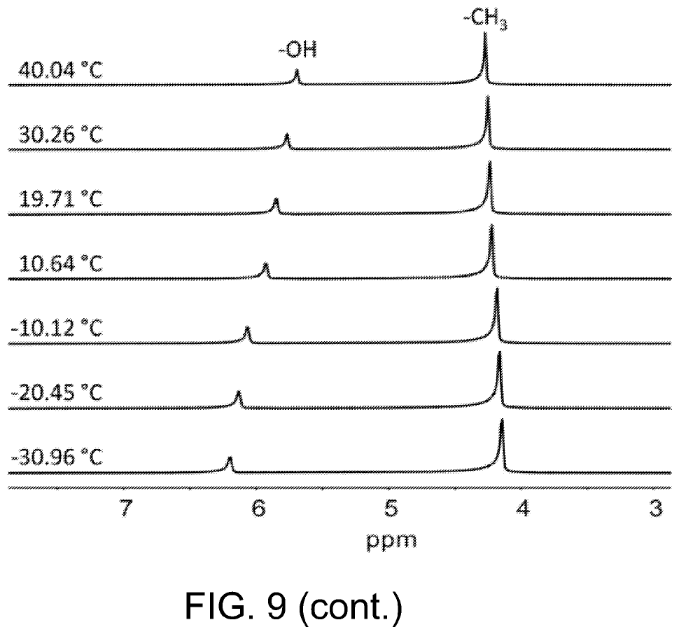

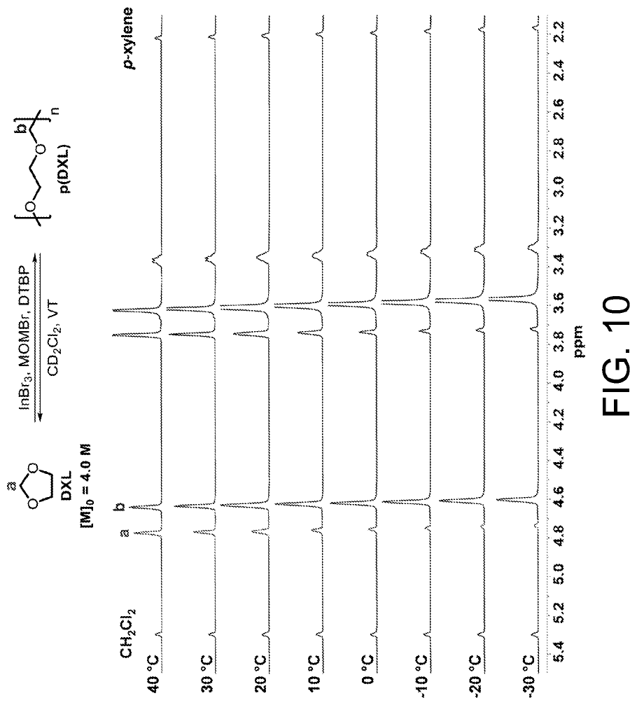

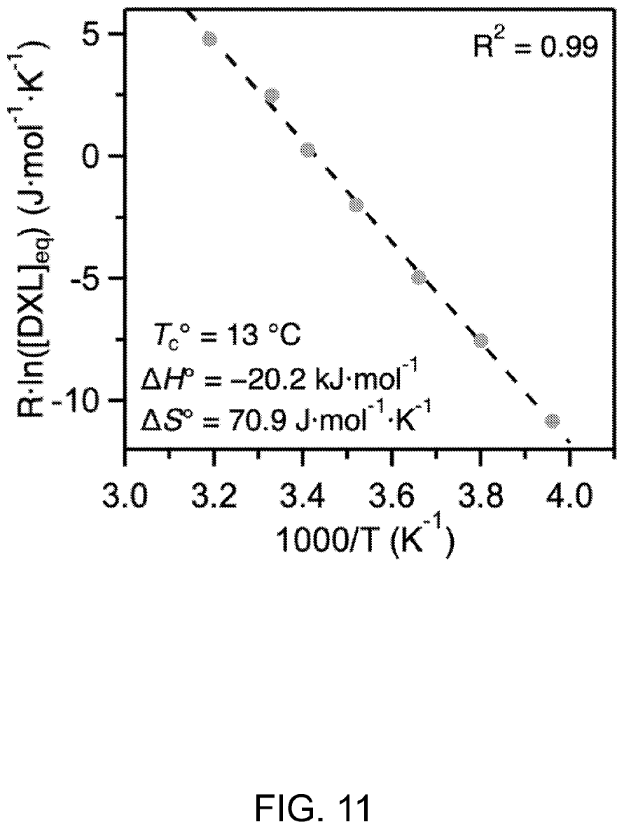

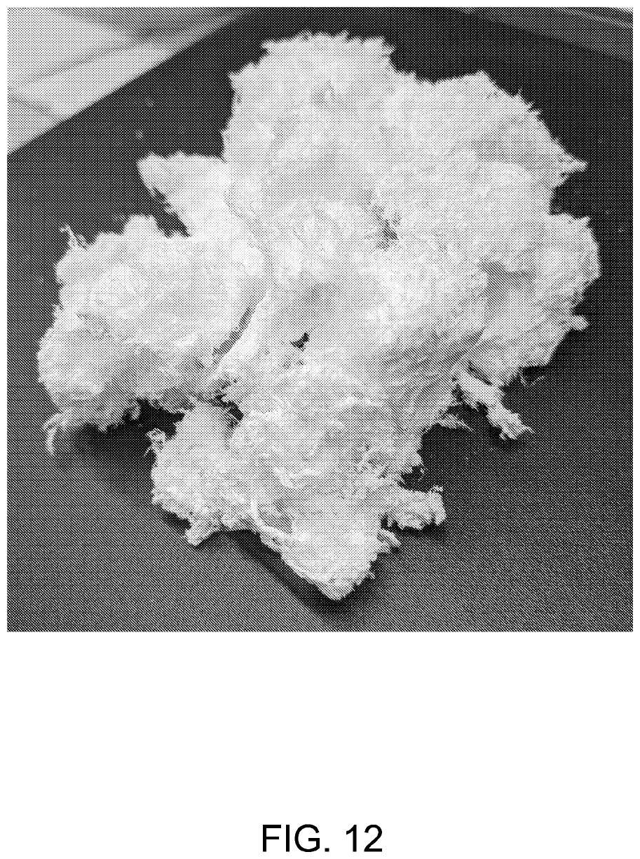

Transacetalization is a common side reaction in CROP of cyclic acetals whereby the acetal linkages of the polymer backbone react with the cationic polymer chain ends due to the similar nucleophilicities of cyclic and acyclic acetals. To test this hypothesis, polymerizations of DXL were performed with increasing equivalents of the acyclic acetal diethoxy methane (DEM) relative to [MOMBr] while holding the monomer to initiator ratio constant ([DXL]₀:[MOMBr]₀=600:1). Increasing [DEM]₀:[MOMBr]₀ incrementally from 0:1 to 20:1 resulted in decreasing M_{n,GPC} values from 58.0 to 2.8 kDa (Table 6) with good agreement between the M_{n,GPC} values and the theoretical molecular weights (M_{n,th}) calculated assuming that [polymer]ₜₒₜ=[MOMBr]₀+[DEM]₀ (Equation 2). Monomodal GPC traces with consistent Ð values (1.63-1.81) were obtained for PDXL produced at each DEM loading, which is indicative that chain transfer to DEM is rapid relative to propagation and reversible. Further evidence of transacetalization during RD-CROP was provided by end-group analysis of low molecular weight samples of PDXL, PDXP, PDXC, PTXC, and PHBD using Matrix assisted laser desorption-ionization time of flight (MALDI-TOF) mass spectrometry. To avoid premature degradation of bromomethyl ether end groups during purification and analysis, reactions were quenched with NaOBn, converting Pn-Br chain ends into Pn-OBn. Chain transfer to polymer during RD-CROP of cyclic acetals initiated by MOMBr and quenched with NaOBn is expected to give a statistical 1:2:1 ratio of MOM-Pn-MOM, MOM-Pn-OBn, and BnO-Pn-OBn end groups. The MALDI-TOF spectra of each polyacetal derivative showed three major populations corresponding to the aforementioned end groups (FIGS. 5-8). While MALD-TOF analysis is not quantitative due to differences in ionization efficiency between samples, a 1:2:1 ratio of relative peak heights corresponding to MOM-Pn-MOM, MOM-Pn-OBn, and BnO-Pn-OBn end groups was observed for most of the polyacetal derivatives.

Living chain end retention during RD-CROP of DXL at room temperature was confirmed by performing sequential monomer additions over 24 h during the InBr₃/MOMBr-mediated polymerization of DXL. Aliquots for GPC analysis were taken following full conversion of each DXL addition and immediately prior to the subsequent DXL addition (FIG. 2E). M_{n,GPC} values increased in proportion to the amount of DXL added (solid lines) while no change in M_{n,GPC} was observed between the polymerization reaching full conversion and the next monomer addition (dashed lines). GPC traces for each aliquot remained monomodal and shifted to lower elution times while dispersity values remained consistent (Ð=1.58-1.71).

Polymer Properties. With an understanding of both the kinetic and thermodynamic principles underlying the controlled polymerization of cyclic acetals, next studied was the thermal stability of each polyacetal derivative. In previous reports, residual catalyst or acid species from incomplete purification can catalyze depolymerization of polyacetals. Because commercial POM is often copolymerized with a small amount of ethylene oxide or DXL—commonly referred to as a “zipper-stopper”—to prevent depolymerization to large amounts of formaldehyde and trioxane, toxicity or environmental problems related to the release of formaldehyde with these polyacetal homopolymers after proper purification was not anticipated. To neutralize or remove any residual acidic species, the polymerizations were quenched with a solution of 0.5 M sodium benzyloxide in THF, diluted with CH₂Cl₂ to reduce the viscosity, stirred over K₂CO₃, and filtered through a basic Al₂O₃ plug. Using this approach, excellent thermal stability (T_{d,}5%≥337° C.) was obtained for all five polyacetal derivatives as measured by thermogravimetric analysis (TGA) (Table 10). Isothermal TGA data obtained at 140° C. shows >99% mass remaining after 2 h for each polyacetal, further supporting the thermal stability of these materials for prolonged times at elevated temperatures (Table 11).

Differential scanning calorimetry (DSC) was used to measure thermal transition temperatures for each polyacetal derivative. PHBD has a glass transition temperature (T_{g}) of 34° C., while PDXL, PDXP, PDXC, and PTXC each possess comparably low T_{g} values from −63 to −61° C. (Table 12). The low T_{g} polymers (PDXP, PDXC, PTXC, and PDXL) are semi-crystalline with melting temperatures (Tₘ) of 11/20, 37, 38, and 58° C., respectively (Table 12). While the Tₘ values of PDXP, PDXC, and PTXC are too close to room temperature for reliable mechanical performance, the Tₘ value of PDXL (58° C.) is comparable to thermal transitions in several commercial materials including poly(lactic acid) (T_{g}=60° C.), poly(ethylene terephthalate) (T_{g}=67° C.), poly(ε-caprolactone) (Tₘ=60° C.), and poly(ethylene oxide) (Tₘ=66° C.). Furthermore, DXL is commonly used as a solvent, is commercially available on large scales, and can be readily synthesized from paraformaldehyde and ethylene glycol, which has potential for bio-sourcing. An initial materials cost analysis reveals promising long-term economic viability for CRM of PDXL, as synthetic optimizations could enable a similar commercialization pathway as PLA. Additionally, further optimization of the polymerization conditions should further reduce this cost. As a result, PDXL was selected as a viable candidate for a CRM-enabled polymer.

Using RD-CROP, PDXL was synthesized on a 10 gram scale with good retention of linear Mn versus [M]₀:[I]₀ to obtain high-molecular weight polymers up to 220 kDa (Table 13). The polymerizations were performed at [DXL]₀=9 M at 0° C. to minimize the amount of solvent required while preventing thermal runaway. After 20 min at 0° C., the reactions were warmed to room temperature and stirred for an additional 2 h at room temperature to ensure they had reached full conversion. The reactions reached high conversion (>95%) isolated yields of ˜70%. After workup with K₂CO₃ and basic Al₂O₃, the polymer was isolated via precipitation into cold diethyl ether and dried at 80° C. under vacuum to give a white, fibrous solid. DSC data shows no variation in the T_{g} or Tₘ values with PDXL samples of differing molecular weight (Table 14) and all molecular weights of PDXL show high thermal stabilities of ˜337° C. (Table 15). Isothermal TGA shows no significant changes in the rate of depolymerization for PDXL of different molecular weights (37.9, 111, 182 or 220 kDa) at 325° C., reaching 75% mass remaining in 22-26 minutes (Table 16).

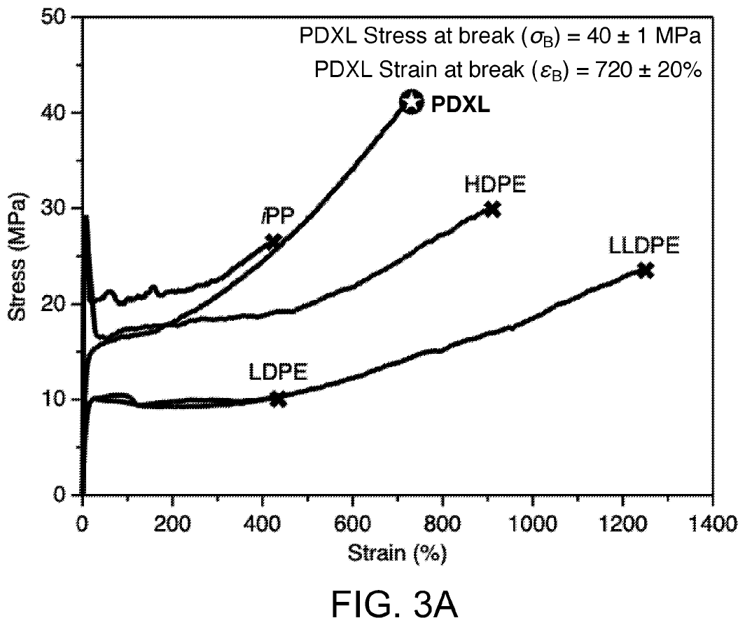

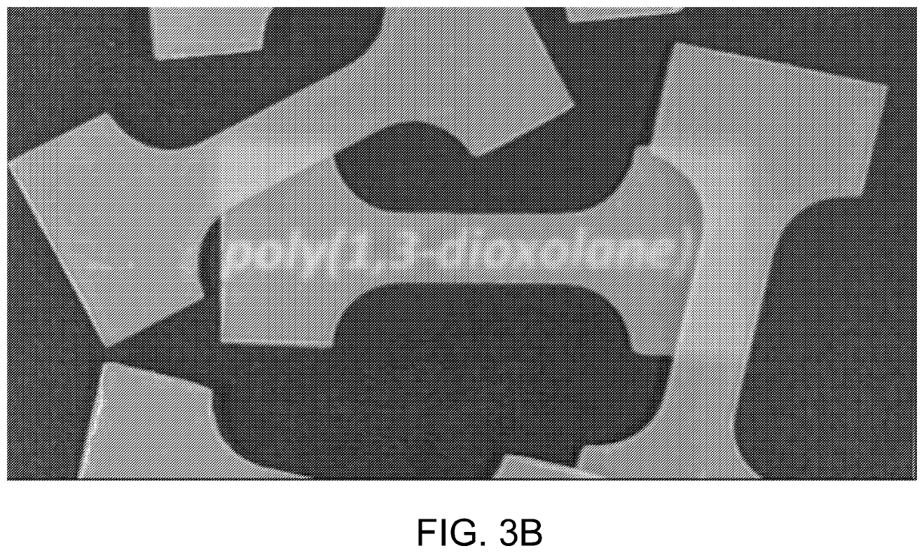

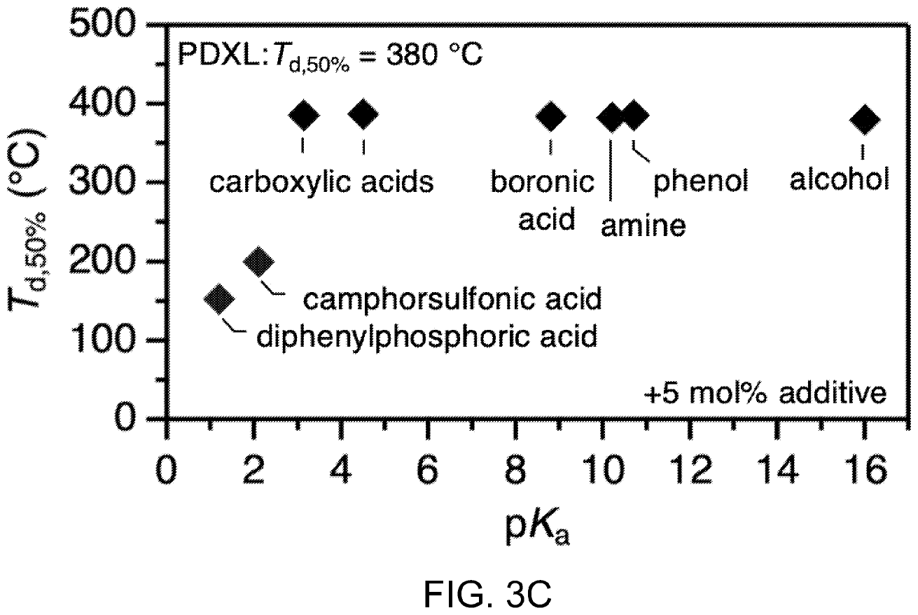

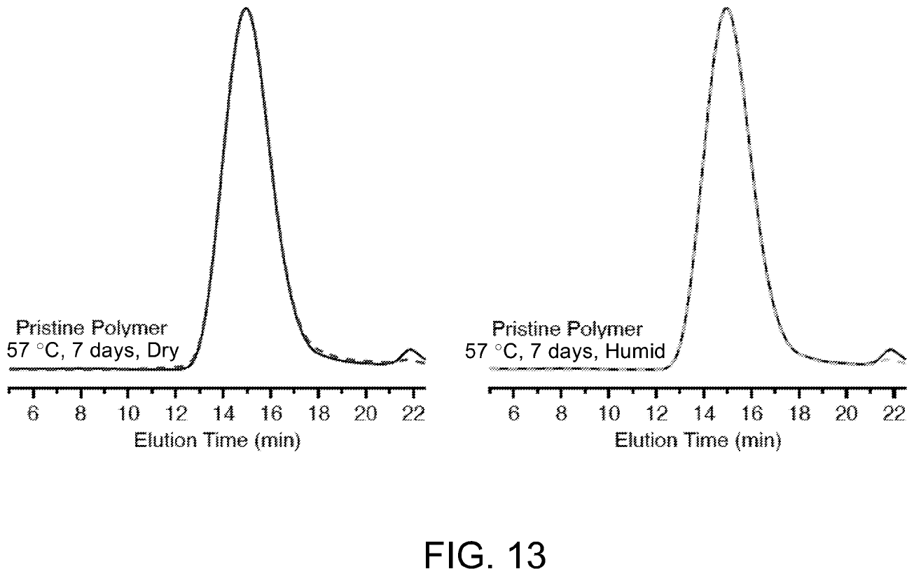

Bulk PDXL was processed in a hot press at 90° C. under 3 MT of pressure for 20 min. to give a colorless, opaque 1 mm thick film (FIG. 3B). The long-term stability of the films under ambient service conditions was studied by placing PDXL (111 kDa, 100 mg) into scintillation vials under either dry or humid conditions at 57° C. for 7 days. The polymer samples show identical GPC traces (FIGS. 13) and ¹H NMR spectra before and after the stability experiment, suggesting no changes in polymer composition even after being subjected to extreme environmental conditions for extended periods of time. The solubility and solvent resistance of PDXL was analyzed by submersing 37.9, 59.9, or 182 kDa PDXL films in hexanes, acetone, methyl ethyl ketone (MEK), THF, CH₂Cl₂, ethanol (EtOH), or H₂O. After 7 and 100 days, the remaining polymer samples were isolated from the solvent, thoroughly dried, and weighed to determine the percent mass remaining. Low molecular weight PDXL (Mₙ=37.9 kDa) showed moderate solvent resistance to many polar and nonpolar solvents including hexanes, acetone, MEK, EtOH with >90% mass remaining after 100 days (Table 17). All molecular weights of PDXL were readily soluble in CH₂Cl₂, but only low molecular weight polymer (Mₙ=37.9 kDa) dissolved slowly in THF, losing 9.3 wt. % over 100 days. Notably, solutions of PDXL in THF can be obtained on heating near the Tₘ (58° C.) and remain homogenous on cooling to room temperature, allowing for GPC analysis in THF. PDXL is known to exhibit some solubility in water, and indeed a piece of 37.9 kDa PDXL disintegrated after just 24 h in water; however, 182 kDa PDXL remained intact in H₂O over 100 days, suggesting entanglement and crystallinity delay solubilization. The molecular weight of both polymers was measured by GPC after 100 days in H₂O and no change was observed, suggesting that the chains can be solvated but not hydrolyzed by neutral H₂O over time.

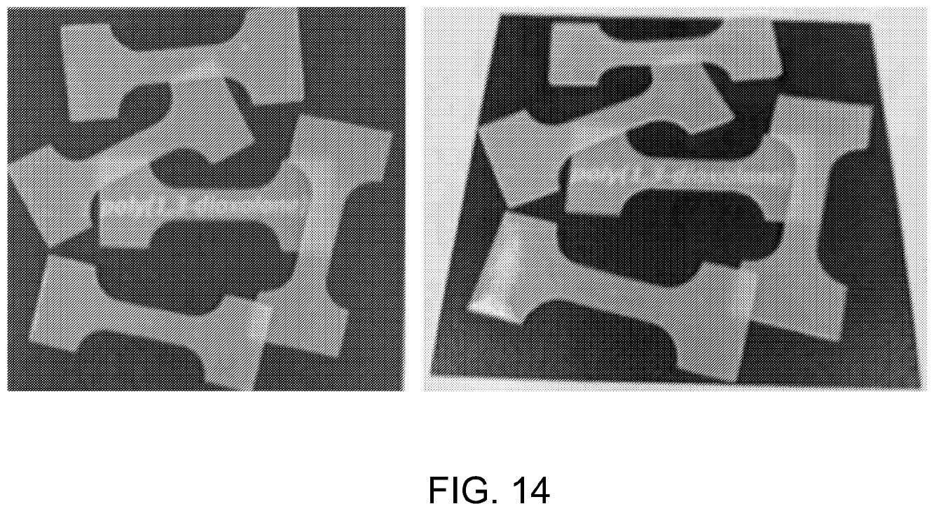

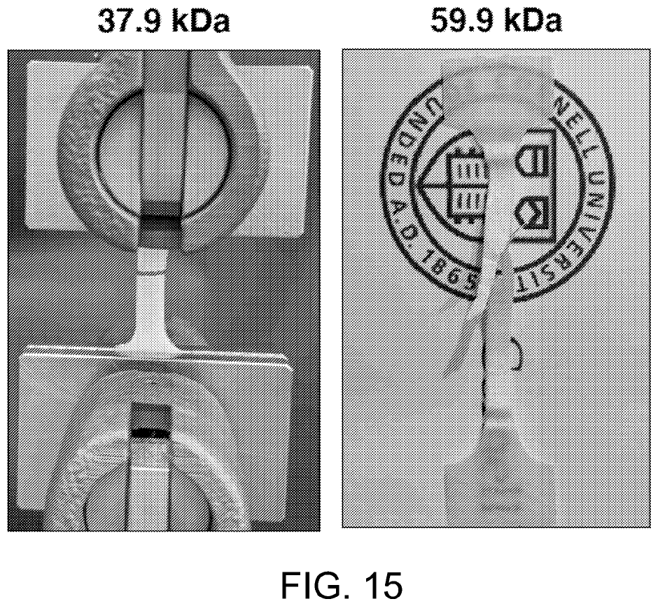

Many linear semi-crystalline thermoplastics like HDPE, LDPE, and iPP are widely used for their tough mechanical properties. Limited by the low molecular weights obtained using prior catalyst systems, PDXL has never been recognized or explored for its mechanical properties. To study PDXL mechanical properties, tensile tests were performed on PDXL samples of increasing molecular weights to estimate the critical molecular weight regime that corresponds to the brittle-ductile transition of PDXL. The PDXL films were cut into dogbones (size ASTM-D1708, 1.0 mm thickness) for tensile testing and data was taken at a strain rate of 5 mm/min for five replicates (FIG. 14 and Table 18). The tensile strength of low molecular weight PDXL (37.9 kDa) was first measured, comparable to the highest previously obtainable molecular weight, and observed brittle properties with tensile stress at break (σ_{B}) of only 13.5±1.3 MPa at 5±0.3% strain (ε_{B}). At 82.3 kDa, PDXL nears entanglement molecular weight and the mechanical properties increase to σ_{B}=33.3±1.2 MPa and ε_{B}=638+45%. At 180 kDa, PDXL showed remarkable mechanical properties with σ_{B}=40.4±1.2 MPa and ε_{B}=717±20%, revealing PDXL as a ductile, tough thermoplastic (FIG. 3A). The mechanical properties of 180 kDa PDXL were compared to data taken from other commodity polymers. As shown in FIG. 3A, PDXL shows improved mechanical properties over LDPE (σ_{B}=10 MPa and ε_{B}=426%), and demonstrates mechanical properties comparable to the most prevalent commodity materials: iPP (σ_{B}=26 MPa and ε_{B}=423%) and HDPE (σ_{B}=30.2±Y MPa and ε_{B}=902%).

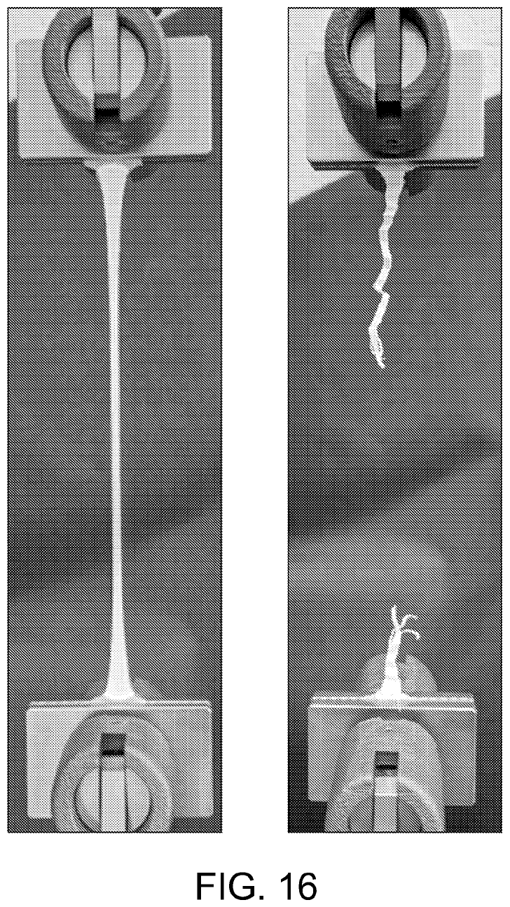

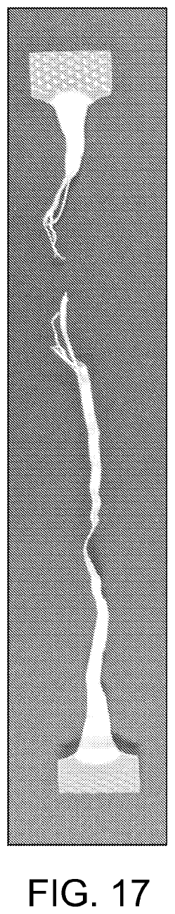

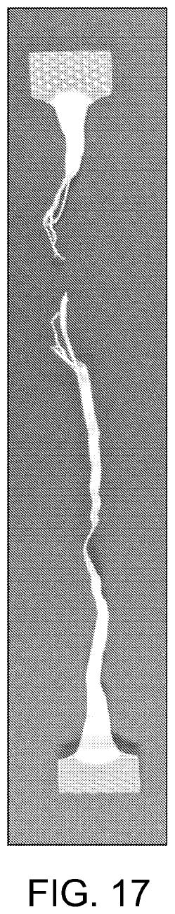

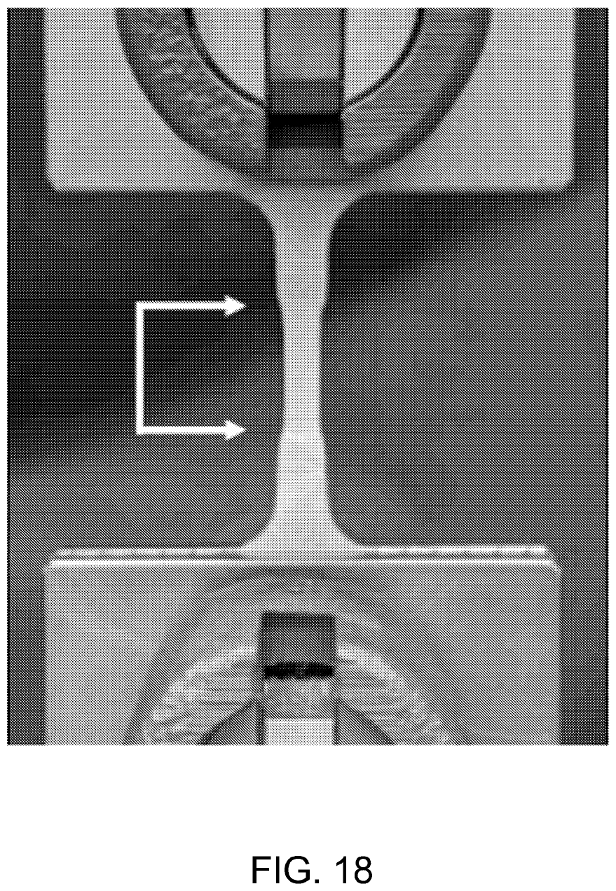

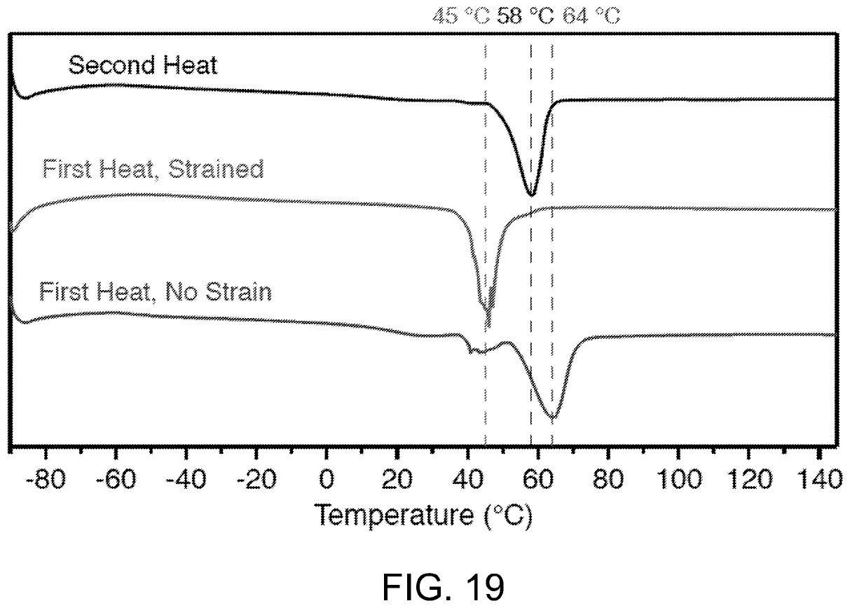

During tensile testing, PDXL undergoes significant strain-induced crystallization, evidenced by the formation of visible white striations that give way to fibrous filaments after fracture (FIGS. 16-17). This important chain alignment contributes to the excellent mechanical properties observed for high molecular weight PDXL. DSC traces of PDXL taken from the first heat before and after strain reveal changes in Tₘ from 64 to 45° C., respectively (FIG. 19). However, DSC traces of the second heat consistently show Tₘ values of 58° C. Further investigation into these trends is needed to understand the relative crystallinity of strained and unstrained materials and will be the focus of a future study. Nevertheless, PDXL was identified as a cost-effective, semi-crystalline polymer with tough mechanical properties comparable to widely used commodity materials like HDPE and iPP.

Chemical Recycling to Monomer. Having demonstrated that PDXL has high thermal stability and mechanical properties comparable to those of commodity polyolefins, the catalyzed depolymerization of PDXL back to DXL monomer was next studied to analyze its suitability for CRM. Importantly, a viable polymer for chemical recycling must be depolymerized efficiently, under an external trigger to produce pure monomer feedstock in high yield. The ceiling temperature of PDXL calculated using VT NMR (T_{c}°=13° C.) indicates that PDXL should depolymerize readily at moderate temperatures using an appropriate catalyst, limited only by the boiling point of the DXL monomer (74° C.).

It was anticipated that PDXL would show good thermal stability in the presence of weak acids and bases, but strong acids would catalyze depolymerization, similar to the stability trends observed for methylene acetal protecting groups. To study the stability of PDXL towards various acids and bases, PDXL samples were doped with a variety of acid or base additives possessing a range of pKₐ and pK_{b} values at 5 mol % (8-15 wt. %) loading (FIG. 3C). High molecular weight additives with low volatility were chosen for use in degradation experiments monitored by TGA to provide the largest temperature window possible before boiling/sublimation of the additive occurred. Thermal stability experiments were performed using dynamic TGA at a heating rate of 10° C./minute and polymer thermal stability was determined using the degradation temperature at 50% mass remaining (T_{d,50%}) to account for any loss of a given additive (Table 19). PDXL degradation was first analyzed in the presence of the Brønsted acids diphenylphosphoric acid (DPP, pKₐ=1.2), camphorsulfonic acid (CSA, pKₐ=2.1), citric acid (pK_{a,1}=2.9), trans-cinnamic acid (pKₐ=4.5), and 4-tolylboronic acid (pKₐ=8.8) (FIG. 54). Strong acids (pKₐ<3) such as DPP and CSA efficiently catalyzed depolymerization of PDXL, affording accessible T_{d,50%} values of 153° C. and 200° C., respectively. However, PDXL doped with weaker acids such as citric acid, which contains three —CO₂H groups (15 mol % —CO₂H to acetal), trans-cinnamic acid, or 4-tolylboronic acid showed excellent thermal stability with T_{d,50%} values near 380° C., matching the T_{d,50%} value of neat polymer (380° C.). These results suggest that weak Bronsted acid-catalyzed degradation is unlikely to occur under normal usage conditions. Lewis acids lithium bis(trifluoromethane)sulfonimide and titanium (IV) isopropoxide also catalyzed PDXL depolymerization (T_{d,50%} values of 323 and 313° C., respectively), albeit much less efficiently than CSA and DPP. Accelerated depolymerization was not observed when representative alcohols 4-tert-butyl phenol and 1-pentadecanol and amine-containing bases octylamine, tributylamine, and 7-methyl-1,5,7-triazabicyclo[4.4.0]decene (MeTBD) were employed, with T_{d,50%} values near 380° C. for each of these additives.

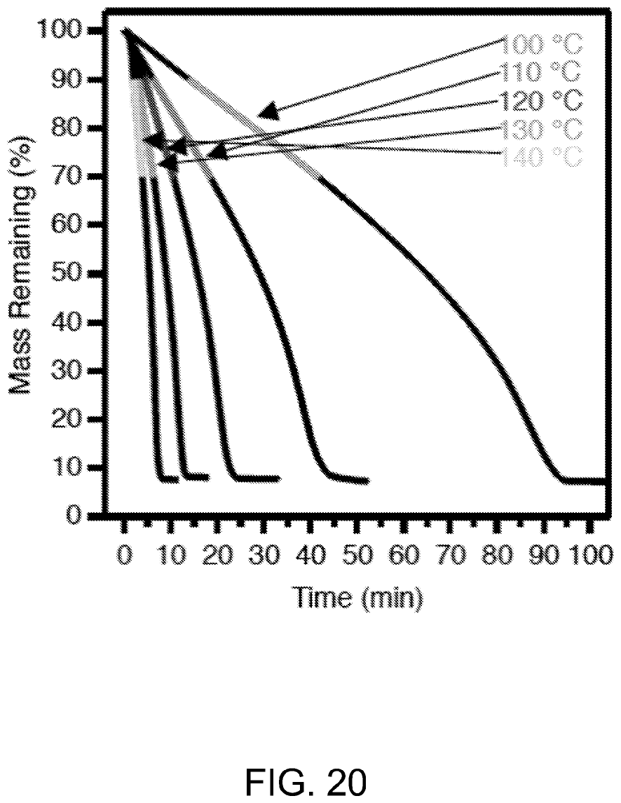

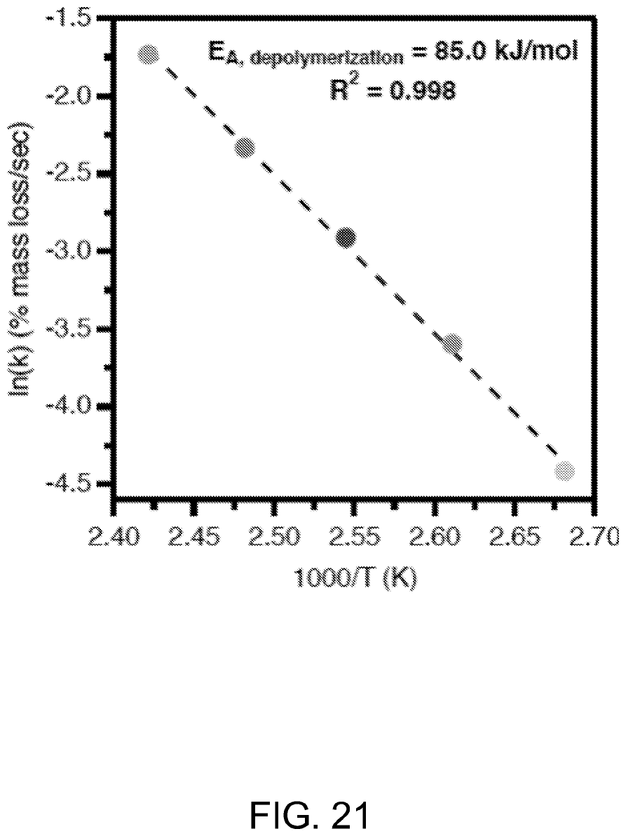

PDXL is remarkably stable towards weak acids and bases but undergoes selective depolymerization in the presence of strong acid catalysts (pKₐ<3). Camphorsulfonic acid (CSA) was selected as a representative strong acid catalyst for the rest of the study because it is an inexpensive, non-volatile, strong acid (pKₐ=2.1) derived from bio-sourced material. The change in T_{d,50%} was measured as a function of CSA loading using 182 kDa PDXL (Table 20). By increasing the CSA loading from 5 to 10 to 15 mol %, the T_{d,50%} decreased from 157 to 108 to 80° C., respectively. Upon increasing the CSA loading to 20 or 25 mol %, T_{d,50%} value plateaus at 73 and 74° C., respectively, coinciding with the boiling point of DXL monomer. The rate of acid-catalyzed degradation decreased slightly with increasing molar mass. At 2 mol % CSA loading and 120° C., 40 kDa PDXL depolymerized rapidly, with 25% mass loss occurring in 6.7 minutes, whereas PDXL of higher molecular weights (110 and 220 kDa) reached 25% mass loss in 9.0 and 10.5 minutes, respectively (Table 21). The depolymerization rate was also measured as a function of temperature of a single molecular weight PDXL sample (127 kDa) doped with 2 mol % CSA. Isothermal TGA experiments were performed in 10° C. increments from 100° C.-140° C. and showed increasing depolymerization rate with increasing temperature as expected (FIG. 20). The rate of degradation (% mass loss·s−1) from 90 to 70% mass remaining was used to determine the Arrhenius activation energy (E_{A}) of CSA-catalyzed PDXL depolymerization to be E_{A}=85.0 kJ/mol (R₂=0.998) (FIG. 21).

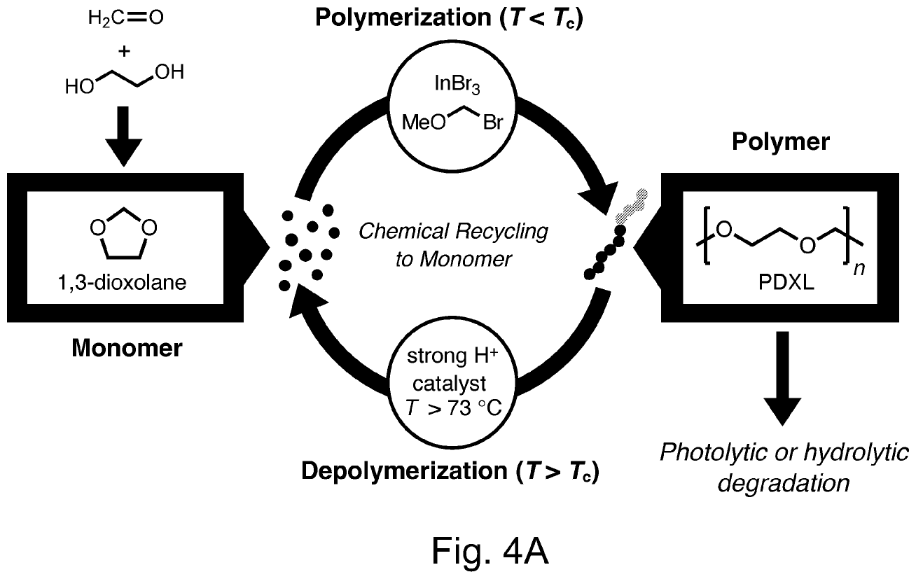

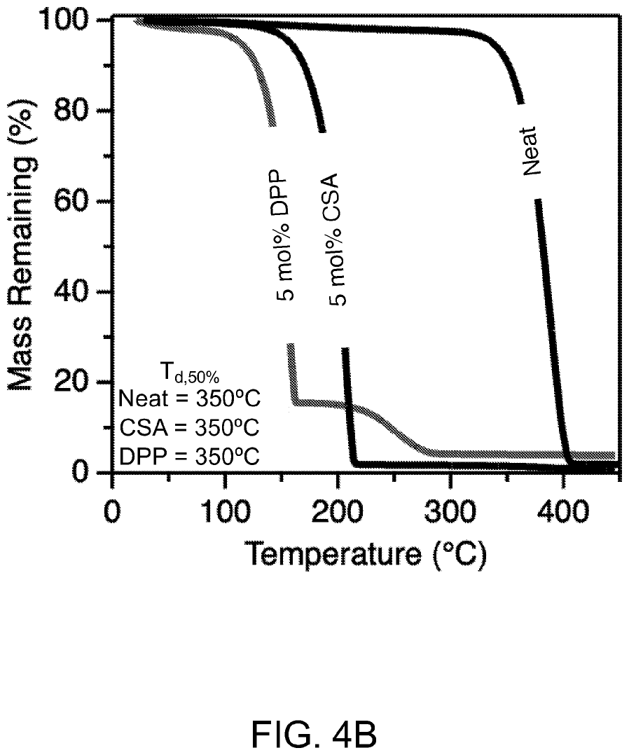

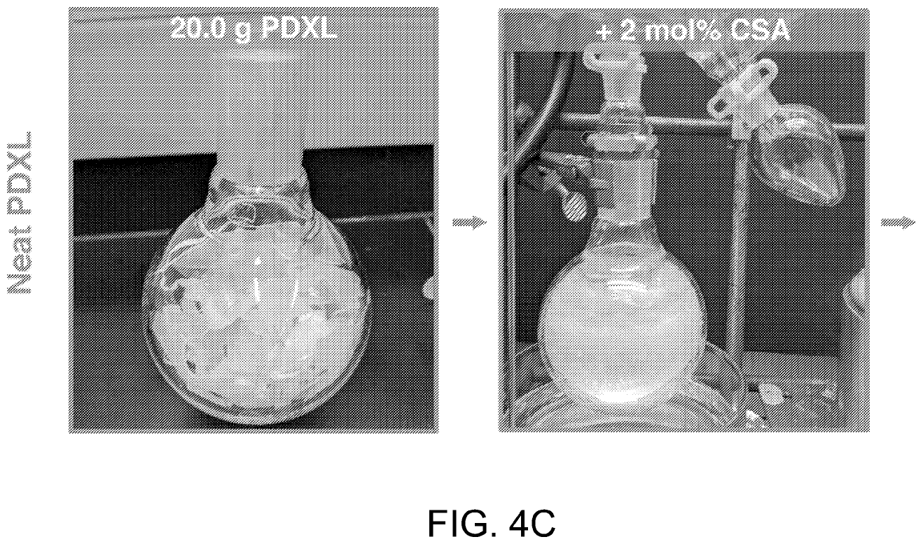

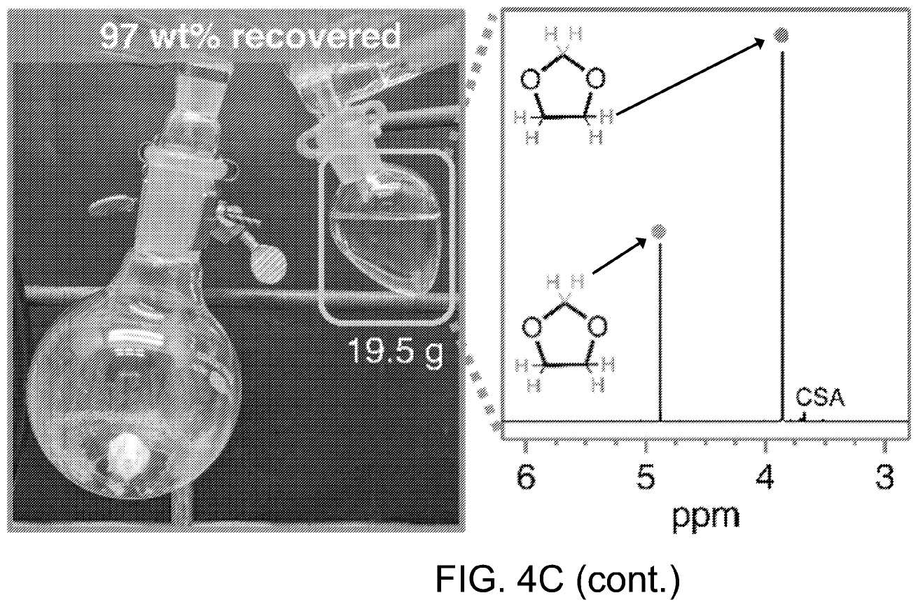

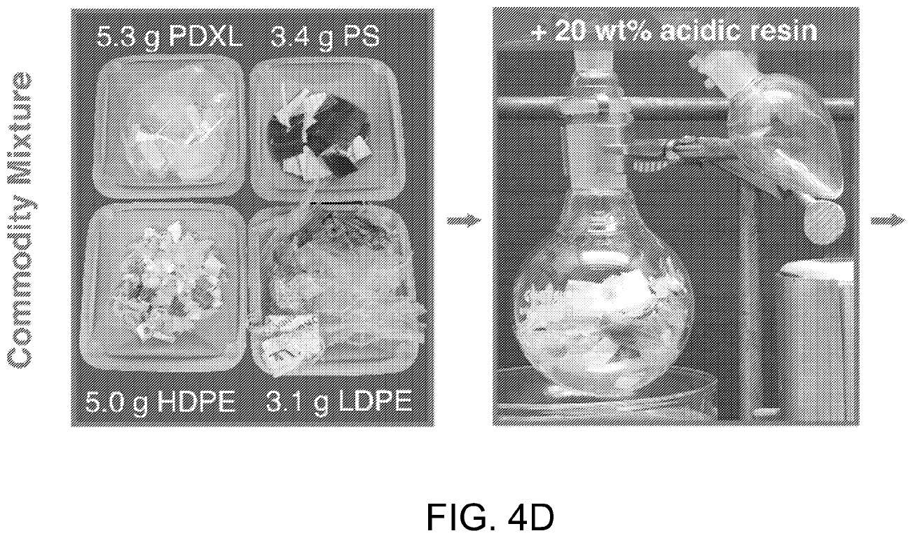

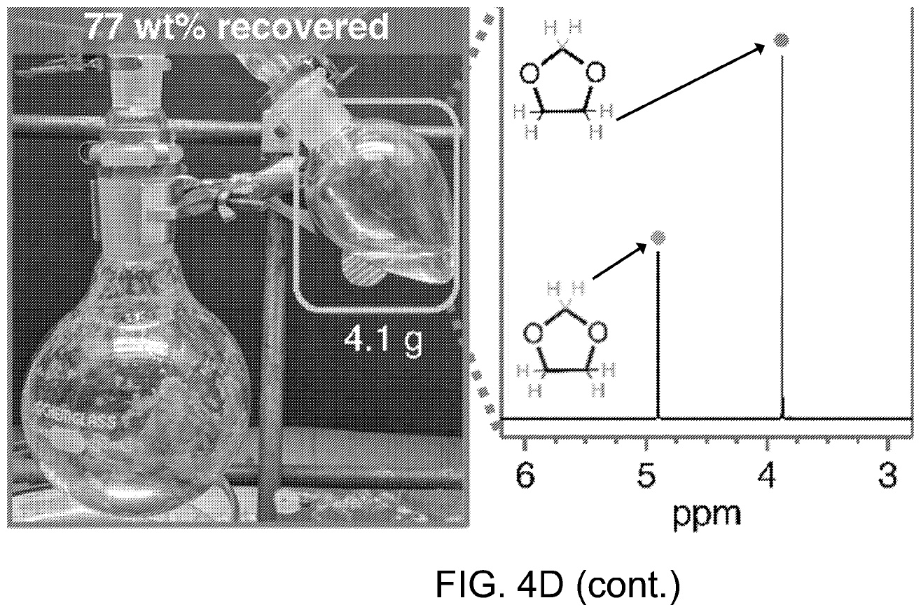

CRM efficacy is dependent upon the depolymerization catalyst, temperature, and monomer collection method. For example, CRM of some polymers is limited by the need for expensive depolymerization catalysts or energy intensive reaction conditions. To make the CRM process amenable to local recycling facilities, an accessible and inexpensive depolymerization process, such as distillation at moderate temperatures, is important in order to mitigate the need for transporting significant amounts of waste to specialized chemical recycling facilities. Based on TGA experiments, PDXL can be readily depolymerized between 73-157° C. in the presence of ≥2 mol % of strong acid. Using these conditions, two methods of large-scale CRM of PDXL were envisioned: (1) homogenous distribution of a small-molecule acid catalyst analogous to the TGA experiments or (2) depolymerization catalyzed by a heterogenous acid resin. PDXL comprising a range of molecular weights (20.0 g; 60-220 kDa) was doped with 2 mol % CSA in CH₂Cl₂ followed by solvent removal in vacuo to give a homogenous dispersion of CSA in PDXL. A short-path distillation was then set up under ambient atmosphere with a dry ice/isopropanol cooled receiving flask. The PDXL/CSA mixture was lowered into an oil bath preheated to 140° C. After heating for 14 minutes, pure DXL began collecting in the receiving flask. The distillation continued for 78 minutes in total, at which point only black residue from degraded CSA remained and 19.5 g (98% yield) of DXL monomer was collected. ¹H NMR spectroscopy was then used to confirm the purity of the DXL monomer (FIG. 4C). The recovered DXL was dried over CaH2 and repolymerized using InBr3/MOMBr-catalyzed RD-CROP in CH₂Cl₂ at room temperature. The resultant polymer shows identical ¹H and ¹³C NMR spectra and mechanical properties (Table 23) as compared to the pristine 80.7 kDa material. It is envisioned that continued optimization of this procedure will yield nearly quantitative recovery of monomer, requiring only a simple distillation at moderate temperature, followed by drying to recover ready-to-polymerize monomer feedstock from polymer.

Waste separation remains the most time and cost-intensive challenge facing current mechanical and chemical recycling approaches, as plastics are collected from mixed waste streams that include different types of plastic, dyes/pigments, plasticizers, stabilizers, and other impurities. A key advantage of CRM is the ability to isolate pure monomer from a complex reaction mixture. To investigate the efficacy of selective polyacetal CRM from mixed commodity polymer feedstocks, a representative mixed plastics waste feedstock comprising HDPE (5.0 g) and LDPE (3.1 g) obtained from recycling facilities, polystyrene (3.4 g) from a disposable cup, and 30 mL of low molecular weight PEO as a viscosity modifier to aid in stirring was combined in around-bottom flask followed by addition of an acid catalyst resin (DOWEX-50, 20 wt. %). Lastly, PDXL of varying molecular weights (3.2 g, 80-180 kDa) was added to the flask and a short path distillation apparatus was fitted under ambient conditions, using a dry ice/acetone bath to cool the receiving flask (FIG. 4D). An initial fraction of clear, colorless DXL monomer (2.3 g) was collected over the first 30 min, at which point the distillation rate slowed significantly. A second portion of PDXL (2.1 g, 80-180 kDa) was then added to the polymer mixture and distillation continued. Once distillation slowed again, a third fraction (0.8 g) was collected during the last 30 min of the experiment (Table 25). Each collected fraction comprised only DXL monomer and H₂O. Excluding the mass of the recovered H₂O, DXL was recovered from the mixed polymer waste feedstock in 77% yield (4.1 g) (Table 25). No dyes, plasticizers, or degradation byproducts resulting from the commodity plastics mixture were detected by ¹H NMR spectroscopy of the reclaimed DXL monomer, suggesting that waste separation is not required prior to CRM of PDXL. Furthermore, it was demonstrated that PDXL can be recycled continuously such that polymer can be consistently added to the reaction mixture and depolymerized and acid catalyst resin. It is anticipated that further optimization of the distillation setup and use of mechanical stirring will further improve the monomer yield. Therefore, it is believed that PDXL is a promising polymer with multiple options for end-of-life depolymerization to either recover material value through monomer collection or avoid environmental waste accumulation through relatively rapid degradation pathways.

In the 21st century, polymer chemists must attend to the plastics problem by designing materials that maintain the functionality of current commodity plastics while mitigating the environmental consequences of plastic build up by enabling strategies such as chemical recycling to monomer. Polyacetals have been identified as class of materials capable of CRM Using NMR experiments to determine AS and AH, the ceiling temperature of bulk polymer was calculated to be T_{c}=13° C. and developed a living RD-CROP of CA monomers via LA catalyst and halomethyl ether initiator that afford polyacetals rapidly at room temperature with excellent molecular weight control. The effects of the catalyst transition metal and halide on monomer reactivity have been demonstrated, ultimately developing an optimized InBr₃ and MOMBr polymerization system which shows excellent livingness over 24 h. This technique was used to study five polyacetals of which, PDXL had the highest Tₘ at 58° C., rendering it viable for commercial application. Furthermore, PDXL can be made rapidly at or near room temperature using an inexpensive monomer and commercially available catalyst and initiator system. Upon scaling up the polymerization, high molar mass PDXL were consistently obtained which demonstrated excellent thermal stability (T_{d,5%}=353° C.) and solvent resistance. The colorless semi-crystalline polymer shows tough mechanical properties, comparable to commodity polymers including HDPE and iPP. While PDXL is stable to many basic, neutral, and weak acid additives, strong acid additives (pKₐ<3) cause rapid degradation of the polymer at relatively low temperatures. Lastly, the efficient chemical recycling of PDXL has been demonstrated, wherein the polymer can be catalytically depolymerized by strong acids to recover pure monomer feedstocks. Chemical recycling experiments show that PDXL can be depolymerized with 2 mol % CSA to obtain 98% nearly pure DXL polymer in just 90 minutes at 140° C. Furthermore, PDXL can be depolymerized to distill monomer directly from an unsorted mixture of commodity polymers in 77% yield at just 140° C. for 120 minutes. PDXL offers a thermally stable, mechanically tough semi-crystalline polymer, which can be depolymerized quickly and in excellent yields at relatively low temperatures with only catalytic strong acids to recover pure monomer feedstocks. Despite initial concerns with its relatively low glass transition temperature and higher cost, PLA has established a place in the commercial market due to its potential for bio-sourcing and bio-degradability. Given the chance, it is believed that poly(1,3-dioxolane) can follow a similar path, creating a circular plastics economy that is imperative to the long-term sustainability of plastic usage.

General Considerations. All manipulations of air and water sensitive compounds were carried out under nitrogen in an MBraun Labmaster glovebox or by using standard Schlenk line techniques. Flash column chromatography was performed using silica gel (particle size 40-64 μm, 230-400 mesh).

Characterization Methods. ¹H and ¹³C NMR—Spectra were recorded on a Bruker AV III HD (¹H, 500 MHz) spectrometer with a broad band Prodigy cryoprobe or Varian INOVA 400 (¹H, 400 MHz) spectrometer. Chemical shifts (δ) for ¹H and ¹³C NMR spectra were referenced to protons on the residual solvent (for ¹H) and deuterated solvent itself (for ¹³C).

High-resolution mass spectrometry (HRMS)—Analyses were performed on a Thermo Scientific Exactive Orbitrap MS system equipped with an Ion Sense DART ion source.

Matrix-Assisted Laser Desorption/Ionization Time-of-Flight Mass Spectroscopy (MALDI-TOF-MS). MALDI-TOF-MS analyses were performed on a Broker Autoflex Speed LRF by the Mass Spectrometry Lab of the School of Chemical Sciences at the University of Illinois, Urbana Champaign. Purified polyacetal samples were dissolved in. Sodium trifluoroacetate was used as the cationization agent and dissolved in THE. The matrix 2,5-dihydroxybenzoic acid was dissolved in THF. Solutions for analysis were prepared by mixing polymer, cationization agent, and matrix solutions in varying ratios and the sample was left to air dry after spotting on a stainless steel MALDI target plate. All spectra were recorded in reflectron mode. The resulting spectra were analyzed using the Polymerix software package.

Gel Permeation Chromatography (GPC)—Analyses were carried out using an Agilent 1260 Infinity GPC System equipped with an Agilent 1260 Infinity autosampler and a refractive index detector. The Agilent GPC system was equipped with two Agilent PolyPore columns (5 μm, 4.6 mm ID), which were eluted with THF at 30° C. at a rate of 0.3 mL/min and calibrated using monodisperse polystyrene standards.

Differential Scanning calorimetry (DSC)—Data was recorded on a TA Instruments Q1000 Modulated Differential Scanning calorimeter using 4-6 mg samples. The samples were heated to 150° C. at 10° C./min and held for 5 min to erase the thermal history. Samples were then cooled to −80° C. at 10° C./min, held at −80° C. for 5 min, and reheated to 150° C. at 10° C./min. The T_{g} and Tₘ values were recorded from the second heating ramp using the maximum value of the derivative of heat flow with respect to temperature.

Tensile Testing—Uniaxial extension experiments were performed on a Shimadzu Autograph AGS-X series instrument with pneumatic grips and a 500 N load cell using ASTM D-1708 standard dogbone-shaped samples (ca. 0.8 mm (T)×3.5 mm (W)×27 mm (L) with gauge lengths measured from grip to grip). Metal grips were used for tensile testing with a grip pressure of 80 psi and a ramp speed of 5 mm/min. Extension to break tests were performed with five replicates per material to report average values and standard deviations for each set. TrapeziumX version 1.5.1 software was used to analyze the resulting data. The Young's modulus was calculated using the slope of the stress-strain curve from 0 to 1% strain. LDPE samples were obtained from Dow (955I) and tensile data was taken from a previous report. Isotactic polypropylene (H314-02Z) and high-density polyethylene (DMDA904) samples were obtained from Dow and tensile data was taken from a previous report.

Thermogravimetric Analysis (TGA)—Thermal gravimetric analysis was obtained using a TA Instruments Q500 Thermogravimetric Analyzer. Analysis was performed on ˜10 mg of a given sample at a heating rate of 10° C./min from 22 to 500° C. under nitrogen gas.

Polymer Film Preparation—Polymer samples were placed in in a 4 in.×4 in. square stainless-steel mold (0.50 mm thickness) between two stainless steel plates with Teflon sheets on the surface. The mold was then placed in a Carver press that had been pre-heated to 90° C. and allowed to equilibrate for 5 min. To remove air bubbles from the samples, pressure was rapidly applied and released from the mold for 30 s. Immediately after, the mold was then placed under 2 metric tons of pressure for 15 min. The heating element was then turned off, water cooling was turned on, and the polymer was cooled to room temperature while still under pressure. The smooth, homogenous polymer film was removed from the mold and dogbones were cut using an ASTM D-1708 cutting die press purchased from Pioneer DieTechs.

Polyacetal Solvolysis Studies—Polymer films (38.7 kDa, 59.9 kDa, 105 kDa) were cut into samples (˜100 mg) and weighed in triplicate. The films were then immersed in each solvent (10 mL), stored in a dark drawer under ambient conditions, and photographed daily with a Google Pixel 3a. At timed intervals, the polymer sample was isolated by filtration and dried under high vacuum at room temperature for 24 hours to remove residual solvent. The final sample weight was then measured in triplicate, and the remaining % mass determined using the average of the three measurements. Materials. General Materials. Solvents for air sensitive reactions were purchased

from Fisher, sparged with ultrahigh purity (UHP) grade nitrogen, and either passed through two columns containing reduced copper (Q-5) and alumina (hexanes, PhMe, and THF) or passed through two columns of alumina (CH₂Cl₂) and dispensed into an oven-dried Straus flask, degassed via three freeze-pump-thaw cycles, and stored under nitrogen over activated 3 A molecular sieves in a glovebox. Otherwise, solvents (EtOAc, Et₂O, hexanes, MeOH, EtOH, CHCl₃, DMF, pentane, heptane) were used as received. Triethylamine was dried over calcium hydride for three days, vacuum transferred to an oven-dried Schlenk flask, degassed by three freeze-pump-thaw cycles, and stored under nitrogen. All other chemicals and reagents, except for polymerization materials, were purchased from commercial sources (Aldrich, Oakwood Chemical, Strem, Advanced ChemBlocks Inc., TCI Chemicals, Alfa Aesar, Acros, and Fisher) and used without further purification.

Polymerization Materials. InCl₃ (Strem, 99.99%), InBr₃ (Strem, 99.999%), GaCl₃ (Acros, 99.99%), AlCl₃ (Fluka, 99%), BCl₃ (Aldrich, 1M in heptane), SbCl₅ (Aldrich, 99%), SnCl₄ (Aldrich, 99%), TiCl₄ (Aldrich, 1M in CH2Cl2), ZrCl₄ (Strem, 99.99%), HfCl₄ (Strem, 99.99%), ZnCl₂ (Fisher, 99.9%), ZnBr₂ (Oakwood, 99.9%), and FeCl₃ (Acros, 99+%) were used without further purification. Chloromethyl methyl ether (Aldrich, 95%) and bromomethyl methyl ether (TCI Chemicals, 97%) were distilled under nitrogen under partial static vacuum, degassed via three freeze-pump-thaw cycles, and stored under nitrogen in a glovebox freezer at −30° C. 2,6-di-tert-butylpyridine (DTBP) was dried over CaH₂ for three days and vacuum distilled, degassed via three freeze-pump-thaw cycles and stored at room temperature under nitrogen over activated 3 Å molecular sieves. All cyclic acetal monomers were dried over CaH₂ for three days, vacuum distilled, and degassed via three freeze-pump-thaw cycles followed by storage at room temperature under nitrogen over 3 Å molecular sieves.

A General Scheme for Monomer Synthesis is illustrated in Scheme 1.

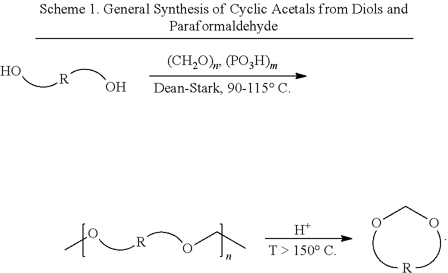

Synthesis of 1,3-dioxepane (DXP)

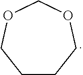

1,4-butanediol (50.0 g, 0.56 mol, 1.00 equiv), paraformaldehyde (6.58 g, 0.56 mol, 1.00 equiv), poly(phosphoric acid) (3.94 g, 28.0 mmol, 0.06 equiv) and cyclohexane (100 mL) were combined in a 250 mL flask with a magnetic stir bar and fitted with a Dean-Stark trap and reflux condenser. The reaction mixture refluxed at 90° C. for 3 h until water collection (˜10 mL) ceased. The mixture was cooled to room temperature and residual cyclohexane removed via rotary evaporation to give a viscous oligomeric mixture. The mixture was distilled at 180° C. under high vacuum, cooling the receiving flask with a dry ice/acetone bath. DXP was obtained as a clear, colorless liquid (25.0 g, 50% yield) with minor 1,4-butanediol impurities. The product was dried over CaH₂ for 3 days (which simultaneously removed the small amount of 1,4-butanediol), vacuum transferred, and degassed via three freeze-pump-thaw cycles. Procedure was based on a previous report. ¹H NMR (500 MHz, CDCl₃): δ4.51 (s, 2H), 3.52 (m, 4H), 1.49 (m, 4H) ppm. ¹³C NMR (125 MHz, CDCl₃): δ94.16, 66.91, 29.14 ppm. HRMS (DART-MS): m/z calculated for C₅H₈O₂ [H]+ 101.0597, found 101.0600.

Synthesis of 1,3-dioxocane (DXC)

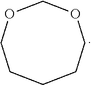

1,5-pentanediol (50.0 g, 0.48 mol, 1.00 equiv) was combined with paraformaldehyde (18.7 g, 0.62 mol, 1.30 equiv), polyphosphoric acid (2.00 g, 14.0 mmol, 0.03 equiv), and heptane (100 mL) in a 250 mL round bottom flask equipped with a stir bar, Dean-Stark adapter, and reflux condenser. The reaction mixture stirred at 115° C. for 3 h until water collection (˜10 mL) ceased. The mixture was cooled to room temperature and excess heptane was removed via rotary evaporation to give a white, solid oligomerized product. The oligomer was distilled under vacuum at 150-180° C. for 2 h and a clear, colorless liquid was collected in a flask cooled with a dry ice/isopropanol bath. An external trap was used to collect excess paraformaldehyde. A second fractional vacuum distillation was performed to remove residual heptane and 1,5-pentanediol and gave clear, colorless liquid (20.5 g, 41% yield). Product was dried over CaH₂ for 3 days, distilled, degassed via 3 freeze-pump-thaw cycles, and stored in a glovebox. Synthesis was carried out based on reported methodology for 1,3,6-trioxocane. ¹H NMR (500 MHz, CDCl₃): δ4.41 (s, 2H), 3.47 (s, 4H), 1.46 (s, 6H) ppm. ¹³C NMR (125 MHz, CDCl₃): δ95.22, 67.79, 29.93, 22.92 ppm. HRMS (DART-MS): m/z calculated for C₆H₁₂O₂ [H]+ 115.0754, found 115.0755.

Synthesis of 1,3,6-trioxocane (TXC)

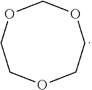

Diethylene glycol (100 g, 0.94 mol, 1.00 equiv), paraformaldehyde (36.8 g, 1.22 mol, 1.30 equiv), polyphosphoric acid (4.01 g, 28.0 mmol, 0.03 equiv), and heptane (160 mL) were combined in a 250 mL flask with a magnetic stir bar and fitted with a Dean-Stark adapter and condenser. The reaction was stirred at 115° C. for 12 h and water (˜15 mL) was collected as the bottom layer in the trap. After cooling the reaction mixture to room temperature, heptane was removed via rotary evaporation to give a cloudy, viscous solution. This oligomerized product was distilled at 150-180° C. under high vacuum into a receiving flask cooled with dry ice/acetone. The crude mixture of diethylene glycol and 1,3,6-trioxocane was then fractionally distilled under high vacuum at 80° C. to give 1,3,6-trioxocane as a clear, colorless liquid (70.0 g, 70% yield). The monomer was dried over CaH₂ for 3 days, distilled, and degassed via three freeze-pump-thaw cycles. Spectral data match previous reports. ¹H NMR (500 MHz, CDCl₃): δ4.57 (s, 2H), 3.50 (s, 8H) ppm. ¹³C NMR (125 MHz, CDCl₃): δ97.91, 72.58, 70.61 ppm. HRMS (DART-MS): m/z calculated for C₅H₁₀O₃ [H]+ 119.0703, found 119.0703.

Synthesis of trans-hexahydro-1,3-benzodioxole (HBD)

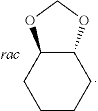

HBD was synthesized according to a literature procedure. A 500 mL round bottomed flask containing magnetic stir bar was charged with trans-1,2-cyclohexanediol (11.6 g, 100 mmol, 1.00 equiv), paraformaldehyde (9.01 g, 300 mmol, 3.00 equiv), and p-toluenesulfonic acid (0.200 g, 1.20 mmol, 0.012 equiv) in 300 mL of a 1:5 (v:v) mixture of EtOH/PhMe. The reaction mixture was heated at 80° C. for 1.5 h followed by fitting the reaction flask with a short path distillation head to remove excess EtOH/H₂O by distillation at atmospheric pressure. Once the distillate temperature exceeded 80° C., static vacuum was applied and the remaining PhMe was removed by distillation, maintaining the temperature at 50° C. The crude mixed acetal was transferred to a 100 mL round bottomed flask and heated in an oil bath at 110° C. under high static vacuum to promote cyclization to the corresponding cyclic acetal. The fraction boiling at ˜40° C. comprising HBD was isolated and redistilled to remove residual PhMe. The pure product was dried over CaH₂ for three days and distilled into a dry Schlenk flask, followed by degassing via three freeze-pump-thaw cycles. The monomer was then stored over activated 3 Å molecular sieves in a glovebox freezer at −30° C. (7.1 g, 55% yield). Spectral data match previous reports. ¹H NMR (500 MHz, CDCl₃): δ4.95 (s, 2H), 2.05 (m, 2H), 1.70 (dt, J=9.42, 1.61, 2H), 1.33 (m, 2H), 1.17 (m, 2H) ppm. ¹³C NMR (125 MHz, CDCl₃): δ94.93, 80.46, 28.55, 23.60 ppm. HRMS (DART-MS): m/z calculated for C₇H₁₂O₂ [H]+ 127.0754, found 127,0753.

Polymer Synthesis. Representative Procedures for Various Cyclic Acetals (1.25 g Monomer) are described below.

Synthesis of Poly(1,3-dioxepane) (PDXP)

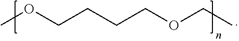

In a glovebox, a 20 mL scintillation vial containing magnetic stir bar was charged with DXP (1.25 g, 1000 equiv) and DTBP (29 μL, 0.05 M) followed by addition of CH₂Cl₂ to give a final volume of (3.06 mL, [DXP]₀=4.0 M). 25 μL of a MOMBr stock solution in CH₂Cl₂ (0.5 M) was then added to the reaction mixture. The vial was sealed with pierceable septum cap and the polymerization initiated while stirring by addition of 250 μL of an InBr₃ stock solution in Et₂O (43 mg/mL). After 20 min the reaction vial was removed from the glovebox and quenched with 0.5 M solution of sodium benzylalkoxide in THF (1.0 mL) followed by stirring at room temperature. The quenched reaction mixture was diluted in CH₂Cl₂ (10 mL) and passed sequentially through two K₂CO₃ pipette plugs and one neutral Al₂O₃ pipette plug to give a clear, colorless solution. The polymer solution was further purified by precipitation into cold hexanes (100 mL) to give a fine white powder that was collected by vacuum filtration and dried under high vacuum overnight. Spectral data match previous reports. ¹H NMR (500 MHz, CDCl₃): δ4.65 (s, 2H), 3.54 (s, 4H), 1.65 (s, 4H) ppm. ¹³C NMR (125 MHz, CDCl₃): δ95.4, 67.6, 26.7 ppm. Mₙ=154 kg/mol; Ð=1.94. T_{g}=−62° C., T_{m,1}=11° C., T_{m,2}=20° C.

Synthesis of Poly(1,3-dioxocane) (PDXC)

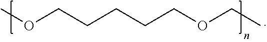

In a glovebox, a 20 mL scintillation vial containing a magnetic stir bar was charged with DXC (1.25 g, 1000 equiv) and DTBP (29 μL, 0.05 M) followed by addition of CH₂Cl₂ to give a final volume of 2.69 mL ([DXC]₀=4.0 M). 21.5 μL of a MOMBr stock solution in CH₂Cl₂ (0.5 M) was then added to the reaction mixture. The vial was sealed with a pierceable septum cap. While stirring, the polymerization was initiated by addition of 250 μL of an InBr₃ stock solution in Et₂O (38 mg/mL). After 20 min the reaction vial was removed from the glovebox and quenched with a 0.5 M solution of sodium benzylalkoxide in THF (1.0 mL) followed by stirring at room temperature. The quenched reaction mixture was diluted in CH₂Cl₂ (10 mL) and passed sequentially through two K₂CO₃ and one basic Al₂O₃ pipette plugs to give a clear, colorless solution. The polymer solution was further purified by precipitation into cold hexanes (100 mL) to give a fine white powder that was collected by vacuum filtration and dried under vacuum overnight. ¹H NMR (500 MHz, CDCl₃): δ4.65 (s, 2H), 3.52 (t, 4H), 1.60 (m, 4H), 1.42 (m, 2H) ppm. ¹³C NMR (125 MHz, CDCl₃): δ95.4, 67.8, 29.7, 23.1 ppm. Mₙ=152 kg/mol; Ð=1.95. T_{g}=−61° C., Tₘ=37° C.

Poly(1,3,6-trioxocane) (PTXC)

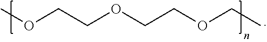

In a glovebox, a 20 mL scintillation vial containing a magnetic stir bar was charged with TXC (1.25 g, 1000 equiv) and DTBP (38 μL, 0.05 M) followed by addition of CH₂Cl₂ to give a final volume of 3.53 mL ([TXC]₀=3.0 M). 10 μL of a MOMBr stock solution in CH₂Cl₂ (90 μL/mL) was then added to the reaction mixture. The vial was sealed with a pierceable septum cap. While stirring, the polymerization was initiated by addition of 250 μL of an InBr₃ stock solution in Et₂O (25 mg/mL). After 20 min, the reaction vial was removed from the glovebox and quenched with 0.5 M solution of sodium benzylalkoxide in THF (1.0 mL) followed by stirring at room temperature. The quenched reaction mixture was diluted in CH₂Cl₂ (10 mL) and passed sequentially through two K₂CO₃ and one basic Al₂O₃ pipette plugs to give a clear, colorless solution. The polymer solution was further purified by precipitation into cold isopropyl alcohol (100 mL) to give a fine white powder that was collected by vacuum filtration and dried under high vacuum overnight. Spectral data match previous reports. ¹H NMR (500 MHz, CDCl₃): δ4.73 (s, 2H), 3.68 (m, 8H) ppm. Mₙ=236.7 kDa; Ð=1.55. ¹³C NMR (125 MHz, CDCl₃): δ95.7, 70.6, 67.0 ppm. T_{g}=−63° C., Tₘ=38° C.

Synthesis of Poly(1,3-hexahydrobenzodioxole) (PHBD)

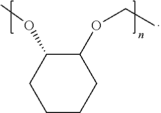

In a glovebox, a 20 mL scintillation vial containing magnetic stir bar was charged with HBD (1.25 g, 1000 equiv) and DTBP (26 μL, 0.05 M) followed by addition of CH₂Cl₂ to give a final volume of 2.44 mL ([HBD]₀=4.0 M). 10 μL of a MOMBr stock solution in CH₂Cl₂ (80 μL/mL) was then added to the reaction mixture. The vial was sealed with a pierceable septum cap. While stirring, the polymerization was initiated by addition of 50 μL of an InBr₃ stock solution in Et₂O (86 μg/mL). After 20 min the reaction vial was removed from the glovebox and quenched with 0.5 M solution of sodium benzylalkoxide in THF (1.0 mL) followed by stirring at room temperature. The quenched reaction mixture was diluted in CH₂Cl₂ (10 mL) and passed sequentially through two K₂CO₃ and one basic Al₂O₃ pipette plugs to give a clear, colorless solution. The polymer solution was further purified by precipitation into cold hexanes (100 mL) to give a fine white powder that was collected by vacuum filtration and dried under vacuum overnight. ¹H NMR (500 MHz, CDCl₃): δ4.84 (m, 2H), 3.51 (s, 1H), 3.42 (s, 1H), 2.00 (m, 2H), 1.64 (s, 2H), 1.30 (s, 4H) ppm. ¹³C NMR (125 MHz, CDCl₃): δ95.9, 92.4, 79.6, 79.6, 34.7, 31.7, 30.3, 29.1, 25.3, 23.7, 22.7, 20.7, 18.8, 14.1, 11.4 ppm. T_{g}=34° C.

A Representative Procedure for Larger Scale PDXL Synthesis (10.0 g Monomer) is illustrated below in Scheme 2.

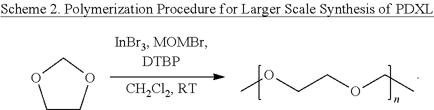

Synthesis of Poly(1,3-dioxolane) (PDXL)

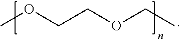

In a glovebox under nitrogen atmosphere, 1,3-dioxolane (10.0 g, 135.0 mmol), di-tert-butyl pyridine (see Table 1), and CH₂Cl₂ were combined in a 100 mL round bottom flask with stir bar to give a final volume of 15.0 mL ([DXL]₀=9.0 M). MOMBr (see Table 1) was added to the reaction mixture using a gas-tight microliter syringe. In a separate vial, InBr₃ (50 mg, 0.142 mmol) was dissolved in anhydrous THF (1.0 mL), and sealed with a pierceable cap. The InBr₃ stock solution and polymerization flask were removed from the glovebox, and the reaction mixture was cooled to 0° C. in an ice bath for at least 10 min. The polymerization was initiated by addition of an InBr 3 stock solution (1.0 mL) directly to the reaction mixture under nitrogen using a gas-tight syringe. After the reaction became too viscous to stir, the reaction mixture was quenched by adding 5 mL of a 1 M solution of sodium benzylalkoxide in THF and mixing vigorously at 0° C. In some cases, mechanical stirring was required due to the high viscosity of the reaction mixture. Once quenched, the reaction mixture was warmed to room temperature and diluted with CH₂Cl₂ (30-150 mL) until reaching a viscosity suitable for precipitation. The reaction mixture was purified by first precipitating into cold diethyl ether (1 L) to give a white fibrous solid. The solid was redissolved in CH₂Cl₂ (30-150 mL) until reaching an appropriate viscosity for vacuum filtration. The solution was then stirred with K₂CO₃ (20.0 g) for 1 h to quench any residual acidic species. The K₂CO₃ was removed via vacuum filtration through a basic alumina and Celite pad, and the polymer was isolated by reprecipitating into cold diethyl ether (1 L). The white fibrous solid was isolated via vacuum filtration, dried at room temperature under vacuum overnight then dried under vacuum at 80° C. for 24 h to remove residual solvent and give a semi-crystalline white solid product. Spectral data match previous reports. ¹H NMR (500 MHz, CDCl₃): δ4.76 (s, 2H), 3.72 (s, 4H) ppm. ¹³C NMR (125 MHz, CDCl₃): δ95.7, 67.0 ppm. T_{g}=−62° C., Tₘ=58° C. δ=1.21 g/mL. See Table 1 for additional reaction conditions, yields, and polymer properties.

TABLE 1
Polymerization conditions and properties for poly(1,3-
dioxolane) synthesized on a larger scale (10 g DXL).

| [M]₀:[I]₀ | MOMBr (μL, mmol) | DTBP (μL, mmol) | Mₙ (kDa) | Ð | % yield |
|---|---|---|---|---|---|
| 200:1 | 55.0, 1.07 | 730, 3.37 | 19.6 | 1.76 | 60 |
| 400:1 | 27.5, 0.34 | 365, 1.69 | 37.9 | 1.76 | 61 |
| 600:1 | 18.4, 0.23 | 243, 1.13 | 57.4 | 1.80 | 55 |
| 800:1 | 13.8, 0.17 | 182, 0.84 | 80.7 | 1.79 | —^{a} |
| 1000:1 | 11.0, 0.14 | 146, 0.68 | 99.4 | 1.76 | 61 |
| 1200:1 | 9.2, 0.11 | 122, 0.56 | 127 | 1.68 | 63 |
| 1600:1 | 6.9, 0.084 | 91, 0.42 | 182 | 1.57 | 51 |

^{a}Yield was not recorded but was similar to other samples.

Other Syntheses. Sodium benzylalkoxide (NaOBn)

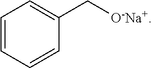

In a glovebox under nitrogen atmosphere, sodium hydride (90%, 2.54 g, 106.1 mmol, 1.2 equiv) was added to a 250 mL round bottom flask equipped with a stir bar, sealed with a rubber septum, and removed from the glovebox. Anhydrous THF (100 mL) was then added under nitrogen to the flask containing NaH and the suspension was cooled to 0° C. using an ice bath. After 10 min, benzyl alcohol (dried over 3 Å molecular sieves, 10.0 g, 92.3 mmol, 1.00 equiv) was added dropwise to the reaction mixture through the septum over several minutes, venting to an oil bubbler. After 30 min, the heterogeneous reaction mixture was removed from the ice bath and allowed to warm to room temperature while stirring overnight. After 16 h, the solids were removed by filtration through an air-free fritted glass filter yielding a homogenous yellow-brown solution of NaOBn in THF. The filtrate was then transferred via cannula to a Schlenk bomb for storage under nitrogen in the dark and used without further purification. NaOBn stock solutions in THF were typically used within 1-3 days of preparation.

Reversible Deactivation Cationic Ring-Opening Polymerization (RD-CROP) Catalyst, Proton Trap, and Initiator Optimization. The following experimental methods were developed to identify catalysts and conditions for controlled RD-CROP using DXL as a representative cyclic acetal (illustrated in Scheme 3). All catalyst screening polymerizations were performed using two catalyst loadings (0.005 M and 0.01 M) according to the following representative procedure.

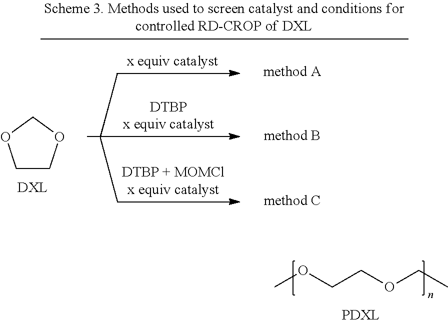

In a glovebox, a 4 mL vial containing a magnetic stir bar was charged with DXL (0.296 g, 300 equiv), DTBP (0 or 5.6 μL (0.005 M)), MOMCl (0 or 20 μL of a 50.6 μL/mL in CH₂Cl₂ stock solution, 0 or 1 equiv) followed by addition of CH2Cl2 to give a final volume of (0.50 mL, [DXL]₀=8.0 M). The polymerization was then initiated by the addition of the amount of the appropriate amount of catalyst stock solution (prepared in Et₂O). Reactions were quenched with 0.1 M TEA in CH₂Cl₂ (0.25 mL) after the specified amount of time, after which aliquots were taken for ¹H NMR (CDCl₃) and GPC (THF) analysis (results shown in Table 2).

Method A (catalyst only) is designed to investigate proton or catalyst-initiated cyclic acetal CROP in the absence of a proton trap (DTBP) and initiator (MOMCl). Polymer formation using method A indicates that either the catalyst directly initiates polymerization and/or catalyst or MOMCl react with adventitious water to generate a protic acid that initiates polymerization. Polymerization under these conditions is considered uncontrolled.

Method B (catalyst+DTBP) is designed to distinguish between catalyst-initiated polymerization and H+-initiated polymerization in the absence of initiator (MOMCl). In this case, DTBP is included as a H+ trap in order to shut down any polymerization initiated by generated protonic acids. Polymer formation using method B therefore indicates that a monomer activated mechanism of cyclic acetal CROP is directly initiated by the catalyst. Catalysts that produce polymer using this method were not considered as viable candidates for the controlled RD-CROP of cyclic acetals. If polymer was formed using method A, but no polymer was formed using method B, the proton trap (DTBP) adequately suppresses H+-initiated polymerization using these catalysts.

Method C (catalyst+DTBP+initiator) is designed to investigate catalyst selectivity for cyclic acetal CROP in the presence of a suitable initiator (MOMCl) and proton trap (DTBP). Catalysts that did not produce polymer using method B, but produced polymer using method C, were considered as selective catalysts for the controlled RD-CROP of cyclic acetals using MOMCl as an initiator. No polymer formation using method C indicates that the catalyst/MOMCl combination is unable to initiate cyclic acetal CROP in 18 h under the reaction conditions investigated in these experiments.

TABLE 2
RD-CROP of DXL catalyst screening using methods A, B, and C.^{a}

| | | | | | equiv | | | | | |
|---|---|---|---|---|---|---|---|---|---|---|
| entry | catalyst | method | [catalyst]₀ | [DTBP]^{b} | MOMCl^{c} | time | | conv. (%)^{d} | Mₙ, _{GPC} (kDa)^{e} | [symbol] ^{e} |
| 1a | InCl₃ | A | 0.005M | 0 | 0 | 18 | h | <1 | — | — |
| 1b | InCl₃ | B | 0.005M | 0.005M | 0 | 18 | h | <1 | — | — |
| 1c | InCl₃ | C | 0.005M | 0.005M | 1 | 18 | h | 85.5 | 34.3 | 1.65 |
| 2a | InCl₃ | A | 0.01M | 0 | 0 | 18 | h | <1 | — | — |
| 2b | InCl₃ | B | 0.01M | 0.005M | 0 | 18 | h | <1 | — | — |
| 2c | InCl₃ | C | 0.01M | 0.005M | 1 | 18 | h | 84.0 | 35.0 | 1.65 |
| 3a | InBr₃ | A | 0.005M | 0 | 0 | 18 | h | <1 | — | — |
| 3b | InBr₃ | B | 0.005M | 0.005M | 0 | 18 | h | <1 | — | — |
| 3c | InBr₃ | C | 0.005M | 0.005M | 1 | 1 | h | 84.0 | 34.3 | 1.66 |
| 4a | InBr₃ | A | 0.01M | 0 | 0 | 18 | h | <1 | — | — |
| 4b | InBr₃ | B | 0.01M | 0.005M | 0 | 18 | h | <1 | — | — |
| 4c | InBr₃ | C | 0.01M | 0.005M | 1 | 10 | min | 84.7 | 38.8 | 1.61 |
| 5a | GaCl₃ | A | 0.005M | 0 | 0 | 18 | h | 80.6 | 280 | 1.67 |
| 5b | GaCl₃ | B | 0.005M | 0.005M | 0 | 18 | h | 77.5 | 625 | 1.56 |
| 5c | GaCl₃ | C | 0.005M | 0.005M | 1 | 1 | min | 76.9 | 32.1 | 1.64 |
| 6a | GaCl₃ | A | 0.01M | 0 | 0 | 10 | min | 54.6 | 234 | 1.69 |
| 6b | GaCl₃ | B | 0.01M | 0.005M | 0 | 18 | h | 84.5 | 172 | 1.77 |
| 6c | GaCl₃ | C | 0.01M | 0.005M | 1 | 1 | min | 77.5 | 29.9 | 1.67 |
| 7a | AlCl₃ | A | 0.005M | 0 | 0 | 18 | h | <1 | — | — |
| 7b | AlCl₃ | B | 0.005M | 0.005M | 0 | 18 | h | <1 | — | — |
| 7c | AlCl₃ | C | 0.005M | 0.005M | 1 | 18 | h | <1 | — | — |
| 8a | AlCl₃ | A | 0.01M | 0 | 0 | 18 | h | <1 | — | — |
| 8b | AlCl₃ | B | 0.01M | 0.005M | 0 | 18 | h | <1 | — | — |
| 8c | AlCl₃ | C | 0.01M | 0.005M | 1 | 18 | h | <1 | — | — |
| 9a | BCl₃ | A | 0.005M | 0 | 0 | 18 | h | <1 | — | — |
| 9b | BCl₃ | B | 0.005M | 0.005M | 0 | 18 | h | <1 | — | — |
| 9c | BCl₃ | C | 0.005M | 0.005M | 1 | 18 | h | <1 | — | — |
| 10a | BCl₃ | A | 0.01M | 0 | 0 | 18 | h | <1 | — | — |
| 10b | BCl₃ | B | 0.01M | 0.005M | 0 | 18 | h | <1 | — | — |
| 10c | BCl₃ | C | 0.01M | 0.005M | 1 | 18 | h | <1 | — | — |
| 11a | SbCl₃ | A | 0.005M | 0 | 0 | 18 | h | <1 | — | — |
| 11b | SbCl₃ | B | 0.005M | 0.005M | 0 | 18 | h | <1 | — | — |
| 11c | SbCl₃ | C | 0.005M | 0.005M | 1 | 18 | h | <1 | — | — |
| 12a | SbCl₃ | A | 0.01M | 0 | 0 | 18 | h | <1 | — | — |
| 12b | SbCl₃ | B | 0.01M | 0.005M | 0 | 18 | h | <1 | — | — |
| 12c | SbCl₃ | C | 0.01M | 0.005M | 1 | 18 | h | <1 | — | — |
| 13a | SbCl₅ | A | 0.005M | 0 | 0 | 10 | min | 84.7 | ^{g} | ^{g} |
| 13b | SbCl₅ | B | 0.005M | 0.005M | 0 | 10 | min | 82.6 | ^{g} | ^{g} |
| 13c | SbCl₅ | C | 0.005M | 0.005M | 1 | 10 | min | 82.6 | 14.6 | 1.68 |
| 14a | SbCl₅ | A | 0.01M | 0 | 0 | 10 | min | 82.6 | ^{g} | ^{g} |
| 14b | SbCl₅ | B | 0.01M | 0.005M | 0 | 10 | min | 82.6 | ^{g} | ^{g} |
| 14c | SbCl₅ | C | 0.01M | 0.005M | 1 | 10 | min | 82.0 | 12.5 | 1.76 |
| 15a | SnCl₄ | A | 0.005M | 0 | 0 | 18 | h | 55.6 | 204 | 1.68 |
| 15b | SnCl₄ | B | 0.005M | 0.005M | 0 | 18 | h | <1 | — | — |
| 15c | SnCl₄ | C | 0.005M | 0.005M | 1 | 1 | h | 58.1 | 23.8 | 1.56 |
| 16a | SnCl₄ | A | 0.01M | 0 | 0 | 18 | h | 72.5 | 143 | 1.91 |
| 16b | SnCl₄ | B | 0.01M | 0.005M | 0 | 18 | h | 58.5 | 155 | 2.06 |
| 16c | SnCl₄ | C | 0.01M | 0.005M | 1 | 1 | h | 68.0 | 26.1 | 1.60 |
| 17a | TiCl₄ | A | 0.005M | 0 | 0 | 18 | h | <1 | — | — |
| 17b | TiCl₄ | B | 0.005M | 0.005M | 0 | 18 | h | <1 | — | — |
| 17c | TiCl₄ | C | 0.005M | 0.005M | 1 | 18 | h | <1 | — | — |
| 18a | TiCl₄ | A | 0.01M | 0 | 0 | 18 | h | <1 | — | — |
| 18b | TiCl₄ | B | 0.01M | 0.005M | 0 | 18 | h | <1 | — | — |
| 18c | TiCl₄ | C | 0.01M | 0.005M | 1 | 18 | h | <1 | — | — |
| 19a | ZrCl₄ | A | 0.005M | 0 | 0 | 18 | h | <1 | — | — |
| 19b | ZrCl₄ | B | 0.005M | 0.005M | 0 | 18 | h | <1 | — | — |
| 19c | ZrCl₄ | C | 0.005M | 0.005M | 1 | 18 | h | <1 | — | — |
| 20a | ZrCl₄ | A | 0.01M | 0 | 0 | 18 | h | <1 | — | — |
| 20b | ZrCl₄ | B | 0.01M | 0.005M | 0 | 18 | h | <1 | — | — |
| 20c | ZrCl₄ | C | 0.01M | 0.005M | 1 | 18 | h | <1 | — | — |
| 21a | HfCl₄ | A | 0.005M | 0 | 0 | 18 | h | <1 | — | — |
| 21b | HfCl₄ | B | 0.005M | 0.005M | 0 | 18 | h | <1 | — | — |
| 21c | HfCl₄ | C | 0.005M | 0.005M | 1 | 18 | h | <1 | — | — |
| 22a | HfCl₄ | A | 0.01M | 0 | 0 | 18 | h | <1 | — | — |
| 22b | HfCl₄ | B | 0.01M | 0.005M | 0 | 18 | h | <1 | — | — |
| 22c | HfCl₄ | C | 0.01M | 0.005M | 1 | 18 | h | <1 | — | — |
| 23a | BiCl₃ | A | 0.005M | 0 | 0 | 18 | h | <1 | — | — |
| 23b | BiCl₃ | B | 0.005M | 0.005M | 0 | 18 | h | <1 | — | — |
| 23c | BiCl₃ | C | 0.005M | 0.005M | 1 | 18 | h | <1 | — | — |
| 24a | BiCl₃ | A | 0.01M | 0 | 0 | 18 | h | <1 | — | — |
| 24b | BiCl₃ | B | 0.01M | 0.005M | 0 | 18 | h | <1 | — | — |
| 24c | BiCl₃ | C | 0.01M | 0.005M | 1 | 18 | h | <1 | — | — |
| 25a | ZnCl₂ | A | 0.005M | 0 | 0 | 18 | h | <1 | — | — |
| 25b | ZnCl₂ | B | 0.005M | 0.005M | 0 | 18 | h | <1 | — | — |
| 25c | ZnCl₂ | C | 0.005M | 0.005M | 1 | 18 | h | 49.0 | 18.6 | 1.56 |
| 26a | ZnCl₂ | A | 0.01M | 0 | 0 | 18 | h | <1 | — | — |
| 26b | ZnCl₂ | B | 0.01M | 0.005M | 0 | 18 | h | <1 | — | — |
| 26c | ZnCl₂ | C | 0.01M | 0.005M | 1 | 18 | h | 77.5 | 28.8 | 1.63 |
| 27a | ZnBr₂ | A | 0.005M | 0 | 0 | 18 | h | <1 | — | — |
| 27b | ZnBr₂ | B | 0.005M | 0.005M | 0 | 18 | h | <1 | — | — |
| 27c | ZnBr₂ | C | 0.005M | 0.005M | 1 | 2 | h | 61.3 | 34.9 | 1.67 |
| 28a | ZnBr₂ | A | 0.01M | 0 | 0 | 18 | h | <1 | — | — |
| 28b | ZnBr₂ | B | 0.01M | 0.005M | 0 | 18 | h | <1 | — | — |
| 28c | ZnBr₂ | C | 0.01M | 0.005M | 1 | 2 | h | 69.9 | 29.2 | 1.63 |
| 29a | FeCl₃ | A | 0.005M | 0 | 0 | 18 | h | 82.0 | 150 | 1.83 |
| 29b | FeCl₃ | B | 0.005M | 0.005M | 0 | 18 | h | 3.0 | 484 | 1.42 |
| 29c | FeCl₃ | C | 0.005M | 0.005M | 1 | 9 | min | 76.9 | 32.0 | 1.65 |
| 30a | FeCl₃ | A | 0.01M | 0 | 0 | 2 | h | 82.6 | 115 | 1.83 |
| 30b | FeCl₃ | B | 0.01M | 0.005M | 0 | 18 | h | 4.7 | ^{f} | ^{f} |
| 30c | FeCl₃ | C | 0.01M | 0.005M | 1 | 6 | min | 80.0 | 31.2 | 1.65 |

^{a}Polymerizations were performed in CH₂Cl₂ at 25° C., [DXL]₀ = 8.0M, 300 equiv DXL.
^{b}DTBP = 2,6-di-tert-butylpyridine.
^{c}MOMCl = chloromethyl methyl ether.
^{d}Determined by ¹H NMR analysis of the crude reaction mixture.
^{e}Determined by GPC in THF, calibrated with polystyrene standards.
^{f}GPC elution time greater than calibration limit.
^{g}GPC elution time below the calibration limit.

$${M}_{nth} = ρ \cdot \frac{{[ M ]}_{0}}{{[ MOMX ]}_{0}} \cdot {MW}_{monomer} + {MW}_{MOMX} \qquad Equation 1$$

where [M]₀ is the initial concentration of monomer, [MOMX]₀ is the initial concentration of initiator, MWₘₒₙₒₘₑᵣ is the molecular weight of the monomer, and MW_{MOMX} is the molecular weight of the initiator.

The effect of InX₃/MOMX Halide Composition on Polymerization Rate is shown below in Scheme 4.

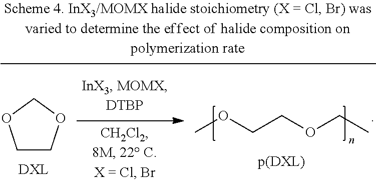

TABLE 3
InX₃/MOMX halide stoichiometry (X = Cl, Br) on DXL polymerization rate.ª

| entry | catalyst | Initiator | [Br]:[Cl] | time | | conv. (%)^{b} | M_{n,th} (kDa)^{c} | M_{n,GPC} (kDa)^{d} | [symbol] ^{d} |
|---|---|---|---|---|---|---|---|---|---|
| 1a | InBr₃ | MOMBr | 4:0 | 10 | s | 20.0 | 4.8 | 7.6 | 1.49 |
| 1b | InBr₃ | MOMBr | 4:0 | 20 | s | 52.4 | 12.5 | 20.3 | 1.57 |
| 1c | InBr₃ | MOMBr | 4:0 | 30 | s | 75.2 | 17.9 | 27.2 | 1.67 |
| 1d | InBr₃ | MOMBr | 4:0 | 40 | s | 77.5 | 18.5 | 29.9 | 1.66 |
| 1e | InBr₃ | MOMBr | 4:0 | 50 | s | 78.1 | 18.6 | 30.4 | 1.66 |
| 1f | InBr₃ | MOMBr | 4:0 | 70 | s | 79.4 | 18.9 | 31.6 | 1.63 |
| 1g | InBr₃ | MOMBr | 4:0 | 80 | s | 79.4 | 18.9 | 32.9 | 1.60 |
| 1h | InBr₃ | MOMBr | 4:0 | 90 | s | 80.6 | 19.2 | 31.3 | 1.65 |
| 1i | InBr₃ | MOMBr | 4:0 | 100 | s | 80.6 | 19.2 | 32.6 | 1.56 |
| 2a | InBr₃ | MOMCl | 3:1 | 25 | s | 11.5 | 2.8 | ^{f} | ^{f} |
| 2b | InBr₃ | MOMCl | 3:1 | 50 | s | 28.4 | 2.8 | 12.4 | 1.51 |
| 2c | InBr₃ | MOMCl | 3:1 | 75 | s | 43.3 | 10.4 | 19.2 | 1.53 |
| 2d | InBr₃ | MOMCl | 3:1 | 100 | s | 63.3 | 15.1 | 27.1 | 1.61 |
| 2e | InBr₃ | MOMCl | 3:1 | 125 | s | 72.5 | 17.3 | 31.0 | 1.63 |
| 2f | InBr₃ | MOMCl | 3:1 | 150 | s | 76.3 | 18.2 | 33.4 | 1.62 |
| 2g | InBr₃ | MOMCl | 3:1 | 175 | s | 77.5 | 18.5 | 33.7 | 1.60 |
| 2h | InBr₃ | MOMCl | 3:1 | 200 | s | 80.0 | 19.1 | 34.0 | 1.64 |
| 2i | InBr₃ | MOMCl | 3:1 | 225 | s | 80.0 | 19.1 | 33.6 | 1.64 |
| 2j | InBr₃ | MOMCl | 3:1 | 250 | s | 80.0 | 19.1 | 32.3 | 1.64 |
| 3a^{e} | InX₃ | MOMX | 2:2 | 30 | s | 5.6 | 1.4 | ^{f} | ^{f} |
| 3b^{e} | InX₃ | MOMX | 2:2 | 60 | s | 12.2 | 3.0 | ^{f} | ^{f} |
| 3c^{e} | InX₃ | MOMX | 2:2 | 90 | s | 20.1 | 4.9 | 7.7 | 1.44 |
| 3d^{e} | InX₃ | MOMX | 2:2 | 150 | s | 41.5 | 9.9 | 14.8 | 1.54 |
| 3e^{e} | InX₃ | MOMX | 2:2 | 180 | s | 50.5 | 12.1 | 18.4 | 1.56 |
| 3f^{e} | InX₃ | MOMX | 2:2 | 210 | s | 62.5 | 14.9 | 23.2 | 1.56 |
| 3g^{e} | InX₃ | MOMX | 2:2 | 270 | s | 75.2 | 17.9 | 26.6 | 1.61 |
| 3h^{e} | InX₃ | MOMX | 2:2 | 330 | s | 79.4 | 18.9 | 28.7 | 1.62 |
| 3i^{e} | InX₃ | MOMX | 2:2 | 390 | s | 80.0 | 19.1 | 27.5 | 1.63 |
| 4a | InCl₃ | MOMBr | 1:3 | 2 | min | 12.4 | 3.1 | 4.8 | 1.39 |
| 4b | InCl₃ | MOMBr | 1:3 | 4 | min | 29.0 | 7.0 | 11.3 | 1.48 |
| 4c | InCl₃ | MOMBr | 1:3 | 6 | min | 47.8 | 11.4 | 17.8 | 1.54 |
| 4d | InCl₃ | MOMBr | 1:3 | 8 | min | 66.2 | 15.8 | 24.6 | 1.57 |
| 4e | InCl₃ | MOMBr | 1:3 | 10 | min | 76.9 | 18.3 | 27.7 | 1.61 |
| 4f | InCl₃ | MOMBr | 1:3 | 12 | min | 80.0 | 19.1 | 28.9 | 1.60 |
| 4g | InCl₃ | MOMBr | 1:3 | 14 | min | 81.3 | 19.4 | 29.6 | 1.63 |
| 4h | InCl₃ | MOMBr | 1:3 | 16 | min | 82.0 | 19.6 | 29.6 | 1.64 |
| 4i | InCl₃ | MOMBr | 1:3 | 18 | min | 82.6 | 19.7 | 30.3 | 1.63 |
| 4j | InCl₃ | MOMBr | 1:3 | 20 | min | 83.3 | 19.9 | 30.5 | 1.66 |
| 5a | InCl₃ | MOMCl | 0:4 | 2 | min | 4.9 | 1.3 | ^{f} | ^{f} |
| 5b | InCl₃ | MOMCl | 0:4 | 4 | min | 10.9 | 2.7 | ^{f} | ^{f} |
| 5c | InCl₃ | MOMCl | 0:4 | 6 | min | 17.5 | 4.3 | 7.2 | 1.49 |
| 5d | InCl₃ | MOMCl | 0:4 | 8 | min | 23.7 | 5.7 | 9.8 | 1.51 |
| 5e | InCl₃ | MOMCl | 0:4 | 10 | min | 30.9 | 7.5 | 12.3 | 1.53 |
| 5f | InCl₃ | MOMCl | 0:4 | 12 | min | 36.5 | 8.8 | 14.7 | 1.55 |
| 5g | InCl₃ | MOMCl | 0:4 | 14 | min | 42.4 | 10.2 | 17.6 | 1.54 |
| 5h | InCl₃ | MOMCl | 0:4 | 16 | min | 48.3 | 11.6 | 19.7 | 1.56 |
| 5i | InCl₃ | MOMCl | 0:4 | 20 | min | 58.8 | 14.1 | 23.5 | 1.59 |
| 5j | InCl₃ | MOMCl | 0:4 | 25 | min | 69.4 | 16.6 | 27.3 | 1.64 |
| 5k | InCl₃ | MOMCl | 0:4 | 30 | min | 76.3 | 18.2 | 30.2 | 1.61 |
| 5l | InCl₃ | MOMCl | 0:4 | 40 | min | 81.3 | 19.4 | 32.3 | 1.62 |

^{a}Polymerizations were performed in CH₂Cl₂ at 22° C., [DXL]₀ = 8M, [MOMX]₀:[InX₃]₀:[DTBP]₀:[DXL]₀ = 1:1:1:320.
^{b}Determined by ¹H NMR analysis of the crude reaction mixture.
^{c}Calculated using Equation 1 assuming [polymer] = [MOMX]₀.
^{d}Determined by GPC in THF, calibrated with polystyrene standards.
^{e}[InX₃]₀:[MOMX]₀ = 2:2 was achieved by using an InX₃ stock solution comprising a 1:1 molar ratio of InBr₃ and InCl₃ and a MOMX stock solution comprising a 1:1 molar ratio of MOMBr and MOMCl.
^{f}Molecular weight was too low to measure accurately.

Effect of [DTBP]₀ (proton trap) on Molecular Weight is shown in Table 4.

TABLE 4
Mₙ vs. [DTBP]₀:[MOMBr]₀ during RD-CROP of DXL.^{a}

| | [DTBP]₀: | time | conv. | M_{n,th} | M_{n,GPC} | | M_{n,GPC}/ |
|---|---|---|---|---|---|---|---|
| entry | [MOMBr]₀ | (min) | (%)^{b} | (kDa)^{c} | (kDa)^{d} | [symbol] ^{d} | M_{n,th} |
| a | 0:1 | 15 | 84.0 | 18.7 | 25.6 | 1.58 | 2.14 |
| b | 1:1 | 15 | 84.0 | 18.7 | 31.3 | 1.63 | 1.68 |
| c | 2:1 | 15 | 84.7 | 18.8 | 31.3 | 1.65 | 1.66 |
| d | 3:1 | 15 | 84.7 | 18.8 | 31.4 | 1.65 | 1.67 |
| e | 4:1 | 15 | 84.0 | 18.7 | 31.1 | 1.66 | 1.66 |
| f | 5:1 | 15 | 84.0 | 18.7 | 32.8 | 1.64 | 1.76 |
| g | 6:1 | 15 | 84.0 | 18.7 | 31.9 | 1.66 | 1.71 |
| h | 7:1 | 15 | 84.0 | 18.7 | 32.0 | 1.64 | 1.71 |
| i | 8:1 | 15 | 84.7 | 18.8 | 31.7 | 1.67 | 1.68 |
| j | 10:1 | 15 | 84.0 | 18.7 | 30.8 | 1.66 | 1.65 |
| k | 50:1 | 15 | 88.5 | 19.7 | 32.6 | 1.72 | 1.66 |

^{a}Polymerizations were performed in CH₂Cl₂ at 25° C., [DXL]₀ = 8M, [MOMBr]₀:[InBr₃]₀:[DTBP]₀:[DXL]₀ = 1:1:X:300.
^{b}Determined by ¹H NMR analysis of the crude reaction mixture.
^{c}Calculated using Equation 1 assuming [polymer] = [MOMBr]₀.
^{d}Determined by GPC in THF, calibrated with polystyrene standards.

RD-CROP Molecular Weight Control is shown in Table 5.

TABLE 5
Mₙ vs. [M]₀:[MOMBr]₀ results for InBr₃-catalyzed RD-CROP of DXL,
DXP, DXC, TXC, and HBD ^{a}.

| | | | time | conv. | | | | |
|---|---|---|---|---|---|---|---|---|
| entry | monomer | [monomer]₀:[MOMBr]₀ ^{b} | (min) | (%) ^{c} | Mₙₜₕ (kDa) ^{d} | Mₙₑₓₚ (kDa) ^{e} | [symbol] ^{e} | Mₙₑₓₚ/Mₙₜₕ |
| 1a ^{f} | DXL | 50:1 | 4 | 84.6 | 5.0 | 4.7 | 1.71 | 0.95 |
| 1b | DXL | 100:1 | 5 | 84.4 | 10.0 | 9.8 | 1.60 | 0.98 |
| 1c | DXL | 200:1 | 6 | 84.2 | 19.9 | 18.8 | 1.58 | 0.94 |
| 1d | DXL | 400:1 | 15 | 85.7 | 40.5 | 40.1 | 1.53 | 0.99 |
| 1e | DXL | 600:1 | 20 | 85.3 | 60.5 | 57.9 | 1.60 | 0.96 |
| 1f | DXL | 1000:1 | 30 | 85.3 | 101 | 101 | 1.58 | 1.00 |
| 2a ^{g} | DXP | 50:1 | 5 | 93.2 | 5.5 | 7.2 | 1.51 | 1.30 |
| 2b | DXP | 100:1 | 5 | 94.3 | 11.1 | 17.3 | 1.65 | 1.55 |
| 2c | DXP | 200:1 | 5 | 94.1 | 22.2 | 32.0 | 1.69 | 1.44 |
| 2d | DXP | 400:1 | 5 | 95.0 | 44.9 | 62.8 | 1.66 | 1.40 |
| 2e | DXP | 600:1 | 5 | 94.8 | 67.2 | 94.4 | 1.68 | 1.41 |
| 2f | DXP | 1000:1 | 5 | 86.8 | 103 | 147 | 1.73 | 1.43 |
| 3a ^{h} | DXC | 50:1 | 1 | 92.0 | 5.4 | 12.2 | 1.61 | 2.24 |
| 3b | DXC | 100:1 | 1 | 85.5 | 10.1 | 23.4 | 1.63 | 2.32 |
| 3c | DXC | 200:1 | 2 | 91.9 | 21.7 | 44.7 | 1.70 | 2.06 |
| 3d | DXC | 400:1 | 3 | 90.2 | 42.6 | 94.7 | 1.74 | 2.22 |
| 3e | DXC | 600:1 | 4 | 85.1 | 60.3 | 136 | 1.74 | 2.25 |
| 3f | DXC | 1000:1 | 5 | 86.3 | 102 | 234 | 1.63 | 2.29 |
| 4a ⁱ | TXC | 50:1 | 1 | 97.0 | 5.7 | 9.6 | 1.55 | 1.67 |
| 4b | TXC | 100:1 | 1 | 96.9 | 11.4 | 17.6 | 1.59 | 1.54 |
| 4c | TXC | 200:1 | 1 | 97.0 | 22.9 | 35.1 | 1.66 | 1.53 |
| 4d | TXC | 300:1 | 1 | 97.0 | 34.4 | 52.9 | 1.67 | 1.54 |
| 4e | TXC | 400:1 | 1 | 96.9 | 45.8 | 71.0 | 1.67 | 1.55 |
| 4f | TXC | 500:1 | 2 | 96.8 | 57.2 | 90.3 | 1.71 | 1.58 |
| 4g | TXC | 750:1 | 2 | 95.8 | 84.9 | 140 | 1.69 | 1.64 |
| 4h | TXC | 1000:1 | 2 | 92.0 | 109 | 181 | 1.69 | 1.66 |
| 5a ^{j} | HBD | 50:1 | 0.5 | 84.2 | 5.4 | 8.8 | 1.59 | 1.64 |
| 5b | HBD | 100:1 | 0.5 | 72.1 | 10.1 | 18.9 | 1.56 | 1.87 |
| 5c | HBD | 200:1 | 1 | 83.7 | 21.5 | 34.1 | 1.51 | 1.59 |
| 5d | HBD | 400:1 | 2 | 83.9 | 43.0 | 62.2 | 1.70 | 1.45 |
| 5e | HBD | 600:1 | 5 | 81.5 | 62.7 | 97.3 | 1.74 | 1.55 |
| 5f | HBD | 1000:1 | 5 | 59.9 | 76.8 | 153 | 1.72 | 2.00 |

ª Polymerizations were performed in CH₂Cl₂ at 22° C., [MOMBr]₀:[InBr₃]₀:[DTBP]₀:[monomer]₀ = X:0.5:5:300.
^{b} [monomer]₀ was held constant and [MOMBr]₀ varied to achieve desired [monomer]₀:[MOMBr]₀.
^{c} Determined by ¹H NMR analysis of the crude reaction mixture.
^{d} Calculated using Equation 1 assuming [polymer] = [MOMBr]₀.
^{e} Determined by GPC in THF, calibrated with polystyrene standards.
^{f} [DXL]₀ = 8.0M.
^{g} [DXP]₀ = 5.0M.
^{h} [DXC] = 5.0M.
^{I} [TXC]₀ = 3.0M.
^{j} [HBD]₀ = 5.0M.

Transacetalization during RD-CROP theoretically occurs according to Scheme 5.

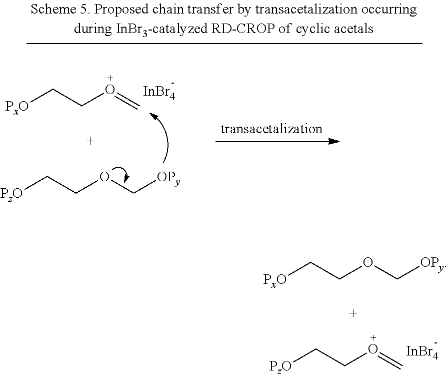

A reaction according to Scheme 6 was performed to test the hypothesis.

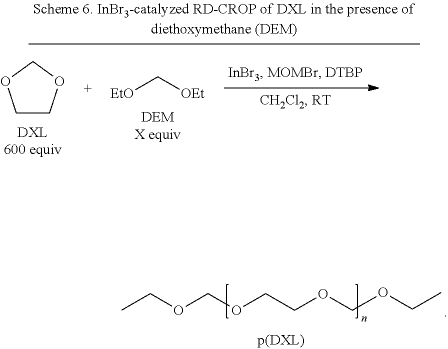

According to Scheme 6, chain transfer to DEM would result in a dependence of M_{n,GPC} on [DXL]₀:([MOMBr]₀+[DEM]₀) according to Equation 2.

$${M}_{nth} = ρ \cdot \frac{{[ M ]}_{0}}{{[ MOMBr ]}_{0} + {[ DEM ]}_{0}} \cdot {MW}_{DXL} \qquad Equation 2$$

In a glovebox, DXL (0.296 g, 4.0 mmol, 600 equiv), DTBP (1.1 μL, 0.005 mmol, 0.01 mM), MOMBr (20 μL of a 27.2 μL/mL stock solution in CH₂Cl₂, 1 equiv), diethoxymethane (0-20 equiv, see Table 6), and CH₂Cl₂ (0.200 mL) were combined in nine separate 4 mL vials with stir bars to give a final [DXL]₀=8.0 M (final total volume=0.50 mL). The polymerizations were then each initiated by addition of 20 μL (0.0025 mmol, [InBr₃]₀=0.005 M) of an InBr₃ stock solution in diethyl ether (44.3 mg/mL) via gas tight syringe and the polymerizations stirred at room temperature for 20 min. Each reaction was quenched by the addition of 0.01 M TEA in CH₂Cl₂ (0.25 mL). Aliquots were taken from each quenched reaction for NMR (CDCl₃) and GPC (THE) analysis. Theoretical molecular weights (M_{n,th}) were calculated based upon Equation 2 assuming 100% chain transfer to diethoxymethane.

TABLE 6
Effect of [DEM]₀:[MOMBr]₀ on Mₙ of InBr₃-catalyzed
RD-CROP of DXL ^{a}.

| | [DEM]₀: | | conv. | M_{n,th} | M_{n,GPC} | | M_{n,GPC}/ |
|---|---|---|---|---|---|---|---|
| entry | [MOMBr]₀ | time | (%) ^{b} | (kDa) ^{c} | (kDa) ^{d} | [symbol] ^{d} | M_{n,th} |
| a | 0:1 | 20 min | 83.3 | 37.0 | 58.0 | 1.66 | 1.57 |
| b | 1:1 | 20 min | 84.0 | 18.7 | 29.9 | 1.64 | 1.60 |
| c | 2:1 | 20 min | 84.7 | 12.5 | 20.5 | 1.64 | 1.64 |
| d | 4:1 | 20 min | 84.7 | 7.5 | 14.4 | 1.63 | 1.92 |
| e | 6:1 | 20 min | 84.7 | 5.4 | 9.0 | 1.68 | 1.67 |
| f | 8:1 | 20 min | 83.3 | 4.1 | 6.9 | 1.74 | 1.67 |
| g | 10:1 | 20 min | 84.7 | 3.4 | 6.0 | 1.74 | 1.74 |
| h | 15:1 | 20 min | 82.6 | 2.3 | 4.0 | 1.75 | 1.75 |
| i | 20:1 | 20 min | 80.6 | 1.7 | 2.8 | 1.81 | 1.65 |

^{a} Polymerizations were performed in CH₂Cl₂ at 25° C., [DXL]₀ = 8.0M, [InBr₃]₀ = 0.005M, [DTBP]₀ = 0.005M, [MOMBr]₀:[DEM]₀:[DXL]₀ = 1:X:600.
^{b} Determined by ¹H NMR analysis of the crude reaction mixture.
^{c} Calculated using Equation 2 assuming [polymer] = [MOMBr]₀ + [DEM]₀.
^{d} Determined by GPC in THF, calibrated with

MALDI-TOF-MS. MALDI-TOF spectra and end-group analyses of PDXL, PDXP, PDXC, and PTXC were performed (shown in FIGS. 5-8). “Livingness” of InBr₃/MOMBr-mediated RD-CROP was demonstrated from sequential addition of monomer to the polymerization mixture as shown in Table 7.

TABLE 7
Polymerization results from sequential monomer additions during the
InBr₃/MOMBr-mediated RD-CROP of DXL^{a}.

| | | DXL | | | | | | |
|---|---|---|---|---|---|---|---|---|
| | | stock | | | | | | |
| ali- | | solution | | total | conv. | Mₙₜₕ | Mₙₑₓₚ | |
| quot | time | added | | equiv | (%)^{c} | (kDa)^{d} | (kDa)^{e} | [symbol] ^{e} |
| T0 | 0 h, | 3.0 | mL | 150 | 0 | — | — | — |
| | 0 min | | | | | | | |
| T1 | 0 h, | — | | — | 84.7 | 16.2 | 9.4 | 1.64 |
| | 5 min | | | | | | | |
| T2 | 2 h, | 3.0 | mL | 300 | 85.5 | 16.1 | 9.5 | 1.61 |
| | 0 min | | | | | | | |
| T3 | 2 h, | — | | — | 84.0 | 33.6 | 18.7 | 1.58 |
| | 5 min | | | | | | | |
| T4 | 4 h, | 3.0 | mL | 450 | 86.2 | 33.9 | 19.2 | 1.66 |
| | 0 min | | | | | | | |
| T5 | 4 h, | — | | — | 84.0 | 49.8 | 28.0 | 1.71 |
| | 10 min | | | | | | | |
| T6 | 6 h, | 3.0 | mL | 600 | 86.2 | 49.4 | 28.7 | 1.58 |
| | 0 min | | | | | | | |
| T7 | 6 h, | — | | — | 84.0 | 78.6 | 37.3 | 1.69 |
| | 15 min | | | | | | | |
| T8 | 8 h, | 3.0 | mL | 750 | 87.0 | 79.0 | 38.7 | 1.63 |
| | 0 min | | | | | | | |
| T9 | 8 h, | — | | — | 85.5 | 93.0 | 47.5 | 1.65 |
| | 20 min | | | | | | | |
| T10 | 23 h, | 3.0 | mL | 900 | 84.7 | 93.3 | 47.1 | 1.62 |
| | 0 min | | | | | | | |

| | | | | | | | |
|---|---|---|---|---|---|---|---|
| T11 | 23 h, | — | — | 83.3 | 113 | 55.5 | 1.68 |
| | 30 min | | | | | | |

^{a}Polymerizations were performed in CH₂Cl₂ at 25° C., [DXL]₀ = 8.0M, [InBr₃]₀ = 0.025M, [DTBP]₀ = 0.025M, [MOMBr]₀ = 0.053M.
^{b}DXL was added to the polymerization mixture as an 8M solution in CH₂Cl₂.
^{c}Determined by ¹H NMR analysis of the crude reaction mixture.
^{d}Calculated using Equation 2 assuming [PDXL] = [MOMBr].
^{e} Determined by GPC in THF, calibrated with polystyrene standards.

Polymerization Thermodynamics. Variable Temperature NMR Measurements. DXL VT NMR Procedure. In a nitrogen-filled glovebox, a 4 mL glass vial with a Teflon cap was briefly charged with 1,3-dioxolane (178 mg, 2.40 mmol, 200 equiv), DTBP (13.5 μL, 0.060 mmol, 5.00 equiv), 20.0 μL of a MOMBr (0.012 mmol, 1.00 equiv) stock solution in CH₂Cl₂ (49.0 μL/mL) and 5 μL of p-xylene (internal standard). Anhydrous CD₂Cl₂ was then added to the vial to reach a volume of 0.55 mL. The solution was then transferred to an oven-dried J. Young NMR tube. The polymerization was initiated by addition of 50.0 μL (0.012 mmol, 1.00 equiv) of an InBr₃ stock solution in Et₂O (85.1 mg/mL) to the J. Young NMR tube to give a final reaction volume of 0.60 mL ([DXL]₀=4 M). The tube was sealed and inverted five times to ensure adequate mixing of the reagents. The NMR tube was then placed into an INOVA 500 MHz NMR spectrometer and ¹H NMR spectra were acquired at the temperatures specified in Table 8. ¹H NMR spectra were recorded starting at the highest temperature, allowing the NMR tube to equilibrate at each temperature for 15 min prior to spectrum acquisition. Equilibrium monomer concentration ([M]_{eq}) was calculated according to Equation 3, where Normalized areaₘₒₙₒₘₑᵣ and Normalized area_{polymer} are the areas of the DXL and PDXL methylene acetal singlets , respectively, when the area of the methyl ¹H's of the internal standard (p-xylene)=1.00.

$${[ M ]}_{eq} = \frac{Normalized {area}_{monomer}}{Normalized {area}_{monomer} + Normalized {area}_{polymer}} \times {[ M ]}_{0} \qquad Equation 3$$

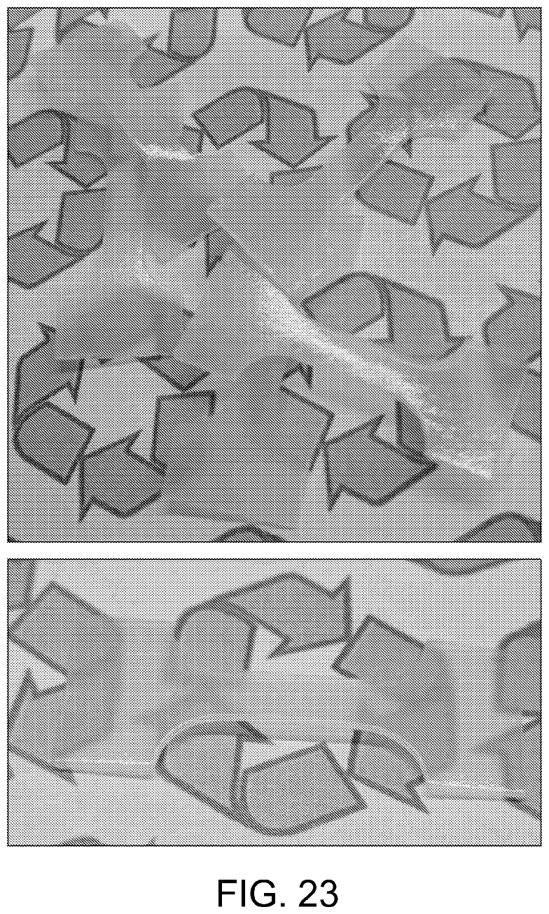

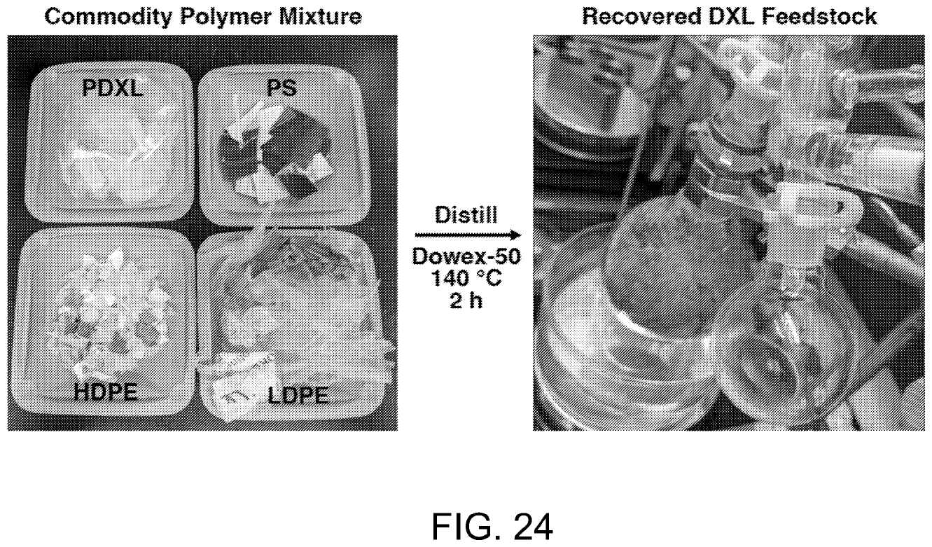

NMR Temperature Calibration. Acquisition temperatures were calibrated using the Vnmrj ‘tempcal’ function by measuring the relative distance in Hz between the C—H and O—H chemical shifts of an anhydrous methanol standard from −30-40° C. in 10° C. increments (FIG. 23).

Manual implementation of the methanol thermometer method can be carried out using the empirical Equation 4 where Δδ is the difference in Hz between the C—H and O—H chemical shifts of a methanol standard at a given temperature.

T(K)=409.0−36.56·Δδ−21.85·(Δδ)² Equation 4

ΔH, ΔS, and T_{c} for DXL measured from VT-NMR.

TABLE 8
Summary of [DXL ]_{eg} vs. T measurements obtained using VT-NMR^{a}.

| | set temp | obs. temp ^{b} | conv. | [DXL]_{eq} ^{c} | R · ln[DXL]_{eq} | 1/T |
|---|---|---|---|---|---|---|
| entry | (° C.) | (° C.) | (%) | (M) | (J · mol⁻¹ · K⁻¹) | (K⁻¹) |
| a | 40.00 | 40.04 | 55.6 | 1.78 | 4.78 | 0.00319 |
| b | 30.00 | 30.26 | 66.3 | 1.35 | 2.49 | 0.00330 |
| c | 20.00 | 19.71 | 74.3 | 1.03 | 0.24 | 0.00341 |
| d | 10.00 | 10.64 | 80.4 | 0.79 | −2.02 | 0.00352 |
| e | 0.00 | 0.00 | 86.2 | 0.55 | −4.95 | 0.00366 |
| f | −10.00 | −10.12 | 89.9 | 0.40 | −7.53 | 0.00380 |
| g | −20.00 | −20.45 | 93.2 | 0.27 | −10.8 | 0.00396 |
| h | −30.00 | −30.96 | 93.5 | 0.26 | −11.2 | 0.00413 |

^{a}[DXL]₀ = 4.0M, [InBr₃]₀ = 0.02M, [MOMBr]₀ = 0.02M, [DTBP]₀ = 0.1M.
^{b} Observed temperature was measured using the MeOH thermometer method.
^{c} [DXL]_{eq} was calculated according to Equation 3.

TABLE 9
Thermodynamic parameters for RD-CROP of DXL from VT-NMR ^{a}.

| monomer | [M]₀ (mol/l) | ΔH (kJ/mol)^{a} | ΔS (J · mol · K⁻¹)^{b} | ΔS° (J · mol · K⁻¹)^{c} | T_{c}° (° C.)^{d} |
|---|---|---|---|---|---|
| DXL | 4.0 | −20.3 | −69.3 | −70.9 | 13.2 |

^{a} Obtained from the slope of R · ln([M]_{eq}) vs 1/T.
^{b} Obtained from the Y-intercept of R · ln([M]_{eq}) vs 1/T. ΔS° = ΔS − ln([M]₀). T_{c}° = (ΔH/ΔS°) − 273.15.

Polymer Properties. Thermogravimetric Analysis (TGA).

TABLE 10
Temperature at 5% mass loss for each polyacetal sample.
Dynamic TGA measurements were taken at 10° C.
per minute from 25-450° C.

| polymer | T_{d, 5%} (° C.) | T_{d, 50%} (° C.) |
|---|---|---|
| PDXL | 337 | 382 |
| PDXP | 365 | 403 |
| PDXC | 392 | 426 |
| PTXC | 347 | 382 |
| PHBD | 339 | 377 |

TABLE 11
Percent mass remaining measured by isothermal TGA after heating
at 140° C. for 2 h for each polyacetal sample. Isothermal
TGA measurements were taken by heating at 50° C./min
to 140° C., then holding the temperature at 140° C. for 2 h.

| polymer | % mass remaining after 2 h at 140° C. |
|---|---|
| PDXL | 99.2 |
| PDXP | 99.9 |
| PDXC | 99.9 |
| PTXC | 99.4 |
| PHBD | 99.0 |

Differential Scanning calorimetry (DSC).

TABLE 12
Glass transition (T_{g}) and melting (Tₘ) temperatures
for all five polyacetals as measured by DSC.

| polymer | T_{g} (° C.) | T_{m, 1} (° C.) | T_{m, 2} (° C.) |
|---|---|---|---|
| PDXL | −62 | 58 | ^{a} |
| PDXP | −62 | 11 | 20 |
| PDXC | −61 | 37 | ^{a} |
| PTXC | −63 | 38 | ^{a} |
| PHBD | 34 | ^{a} | ^{a} |

^{a} No thermal transition observed for this material.

Larger Scale PDXL Characterization . Gel Permeation Chromatography (GPC).

TABLE 13
M_{n, GPC} and Ð values of PDXL synthesized at varying
monomer to initiator ratios on a 10.0 g DXL scale.

| [M]₀:[I]₀ (equiv) | Mₙ (kDa) | Ð |
|---|---|---|
| 200:1 | 19.6 | 1.76 |
| 400:1 | 37.9 | 1.76 |
| 600:1 | 57.4 | 1.80 |
| 800:1 | 80.7 | 1.79 |
| 1000:1 | 99.4 | 1.76 |
| 1200:1 | 127 | 1.68 |
| 1600:1 | 182 | 1.57 |
| 2000:1 | 220 | 1.63 |

Differential Scanning calorimetry.

TABLE 14
T_{g} and Tₘ values for poly(1,3-dioxolane) with varying
M_{n, GPC} synthesized on a large scale (10.0 g of monomer).

| Mₙ (kDa) | Ð | T_{g} (° C.) | Tₘ (° C.) |
|---|---|---|---|
| 37.9 | 1.76 | −61 | 57 |
| 59.9 | 1.83 | −62 | 59 |
| 82.4 | 1.72 | −61 | 58 |
| 111 | 1.91 | −62 | 58 |
| 127 | 1.68 | −62 | 58 |
| 182 | 1.57 | −62 | 59 |
| 220 | 1.64 | −62 | 58 |

Thermogravimetric Analysis.

TABLE 15
Temperature at 5% mass loss measured for neat PDXL samples
of various molecular weights using dynamic TGA heated
at 10° C./min under a nitrogen atmosphere. No change in
T_{d, 5%} was observed with increasing Mₙ.

| Mₙ (kDa) | T_{d, 5%} (° C.) |
|---|---|
| 39.9 | 347 |
| 59.9 | 345 |
| 82.3 | 347 |
| 111 | 337 |
| 127 | 344 |
| 183 | 346 |
| 220 | 349 |

TABLE 16
Time to reach 75% mass remaining for neat PDXL samples
of varying M_{n, GPC} measured by isothermal TGA at
325° C. No significant changes in decomposition
time were observed at different molecular weights.

| | Time to | % mass remaining |
|---|---|---|
| M_{n, GPC} (kDa) | 75% (min) | @ t = 120 min. |
| 37.9 | 22.6 | 12.0 |
| 111 | 24.0 | 7.9 |
| 183 | 25.9 | 12.0 |
| 220 | 24.9 | 9.7 |

Thermal and Solvent Stability. Thermal Stability Experiment—Polymer samples (100 mg, M_{n,GPC}=99.3 kDa, Ð=1.75) were placed in a 20 mL scintillation vial that had either been flame-dried and sparged with nitrogen for 5 minutes to obtain a dry environment or that contained two drops of H₂O to mimic a humid environment and sealed with an air-tight cap. The vials were placed in an aluminum heating block on a hot plate at 57° C., the hottest temperature ever reached in the United States in Death Valley, CA on Jul. 10, 1913. Samples were maintained at 57° C. for one week and NMR and GPC analyses compared before and after heating.

TABLE 17
Percent mass remaining after PDXL samples was soaked in different
solvents under ambient conditions for 7 or 100 days.

| PDXL | hexanes | acetone | MEK^{a} | THF^{b} | CH₂Cl₂^{c} | ethanol | H₂O |
|---|---|---|---|---|---|---|---|
| 37.9 kDa- | 99.4 | 94.9 | 97.6 | 91.0 | 0 | 98.3 | 42.0 |
| 7 days | | | | | | | |
| 59.9 kDa- | 99.7 | 97.8 | 98.5 | 91.7 | 0 | 99.0 | 41.3 |
| 7 days | | | | | | | |
| 182 kDa- | 99.6 | 98.1 | 98.9 | 98.0 | 0 | 99.0 | 83.3 |
| 7 days | | | | | | | |
| 37.9 kDa- | 99.2 | 94.9 | 97.7 | 90.7 | 0 | 98.5 | 37.9 |
| 100 days | | | | | | | |
| 59.9 kDa- | 99.2 | 97.7 | 98.6 | 97.7 | 0 | 98.9 | 79.6 |
| 100 days | | | | | | | |
| 182 kDa- | 99.8 | 97.3 | 98.3 | 90.6 | 0 | 99.0 | 35.9 |
| 100 days | | | | | | | |

^{a}Methyl ethyl ketone.
^{b}Tetrahydrofuran.
^{c}Dichloromethane.

Tensile Testing.

TABLE 18
Average stress at break, strain at break, and Young's
modulus values for PDXL of varying molecular weights.

| Mₙ | Average Stress | Average Strain | Average |
|---|---|---|---|
| (kDa) | (MPa)^{a} | (%)^{b} | E′^{c} |
| 37.9 | 13.5 ± 1.3 | 5.0 ± 0.3 | 0.41 ± 0.03 |
| 59.9 | 21.6 ± 0.7 | 439 ± 38 | 0.29 ± 0.08 |
| 82.3 | 33.3 ± 1.2 | 638 ± 45 | 0.28 ± 0.01 |
| 84.4 | 30.3 ± 2.7 | 577 ± 52 | 0.28 ± 0.02 |
| (Recovered)^{d} | | | |
| 111 | 36.2 ± 2.3 | 607 ± 44 | 0.44 ± 0.01 |
| 127 | 35.8 ± 1.3 | 700 ± 40 | 0.37 ± 0.05 |
| 183 | 40.4 ± 1.2 | 717 ± 20 | 0.34 ± 0.05 |

^{a}Stress at break.
^{b}Strain at break.
^{c}Young's modulus calculated as the slope from 0 to 1% strain.
^{d}Polymer was synthesized from DXL monomer that was recovered during depolymerization experiments.

Chemical Recycling Experiments. TGA of Polymer with Additives.

TABLE 19
Additives, pKₐ values, wt. % additive and decomposition
temperature (to 50% mass) for PDXL with 2 mol % additive
when heated at 10° C./min from 40-450° C.

| | | | T_{d, 50%} |
|---|---|---|---|
| additive | pKₐ | wt % | (° C.) |
| camphorsulfonic acid (CSA) | 1.2 | 13.6 | 200 |
| diphenylphosphoric acid | 1.8 | 14.4 | 153 |
| citric acid | 3.13, 4.76, 6.39^{a} | 11.5 | 386 |
| trans-cinnamic acid | 4.5 | 9.1 | 387 |
| 4-tolylboronic acid | 8.8 | 9.3 | 384 |
| 4-t-butylphenol | 10.2 | 9.3 | 383 |
| 1-pentadecanol | 16 | 13.3 | 380 |
| octylamine | 10.7 | 8.0 | 386 |
| tributylamine | [10.8]^{b} | 11.0 | 383 |
| 7-methyl-1,5,7- | [17.3]^{b} | 9.4 | 379 |
| triazabicyclo[4.4.0]decene | | | |
| LiTFSI | — | 16.2 | 323 |
| Ti(IV) isopropoxide | — | 16.1 | 313 |

^{a}pKa values (in H₂O) for the first, second, and third acidic protons, respectively.
^{b}pKₐ value listed corresponds to the conjugate acid.
^{c}Degradation temperatures (T_{d, 50%}) were recorded at 50% weight loss such that the recorded temperature was attributed to PDXL degradation rather than mass loss from a given additive.

TABLE 20
Data from dynamic (10° C./min) and isothermal (120° C.) TGA
of PDXL with 5 to 15 mol % of added camphorsulfonic acid (CSA).

| added camphorsulfonic | weight % | % | T_{d, 50%} |
|---|---|---|---|
| acid | added | remaining | (° C.)^{a} |
| 5 mol % | 13.6 | 2 | 157 |
| 10 mol % | 23.9 | 0 | 108 |
| 15 mol % | 32.0 | 32 | 80 |
| 20 mol % | 38.5 | 38 | 73 |
| 25 mol % | 43.7 | 42 | 74 |

^{a} Degradation temperatures (T_{d, 50%}) were recorded at 50% weight loss such that the recorded temperature was attributed to PDXL degradation rather than mass loss from a given additive.

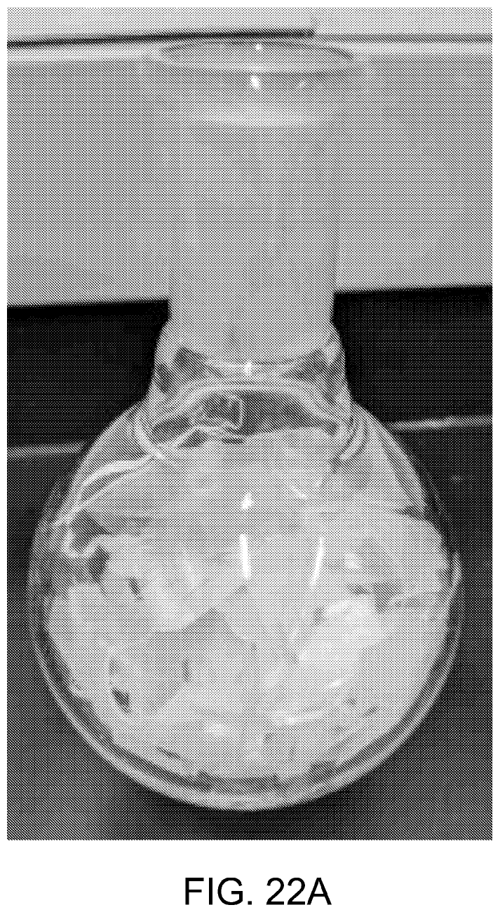

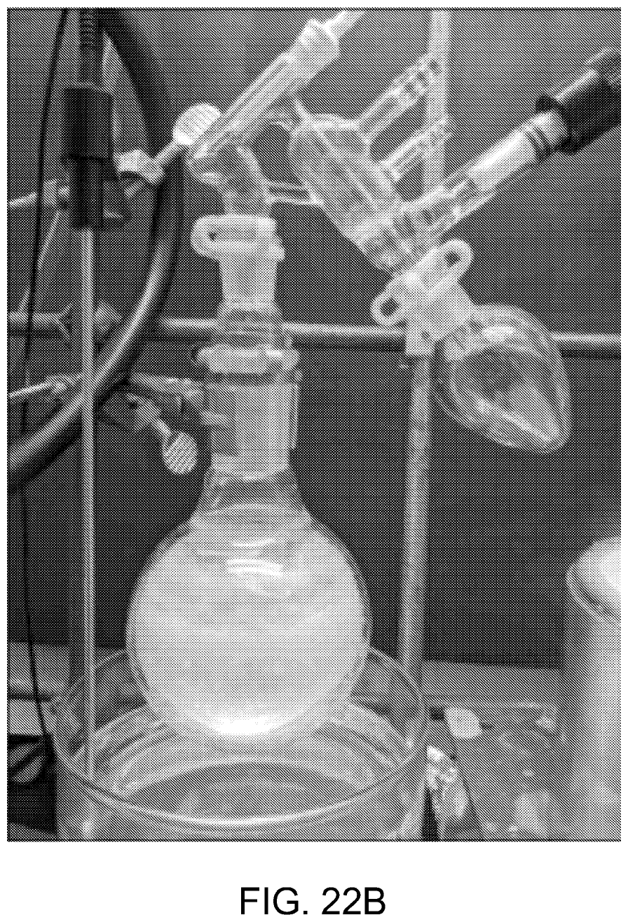

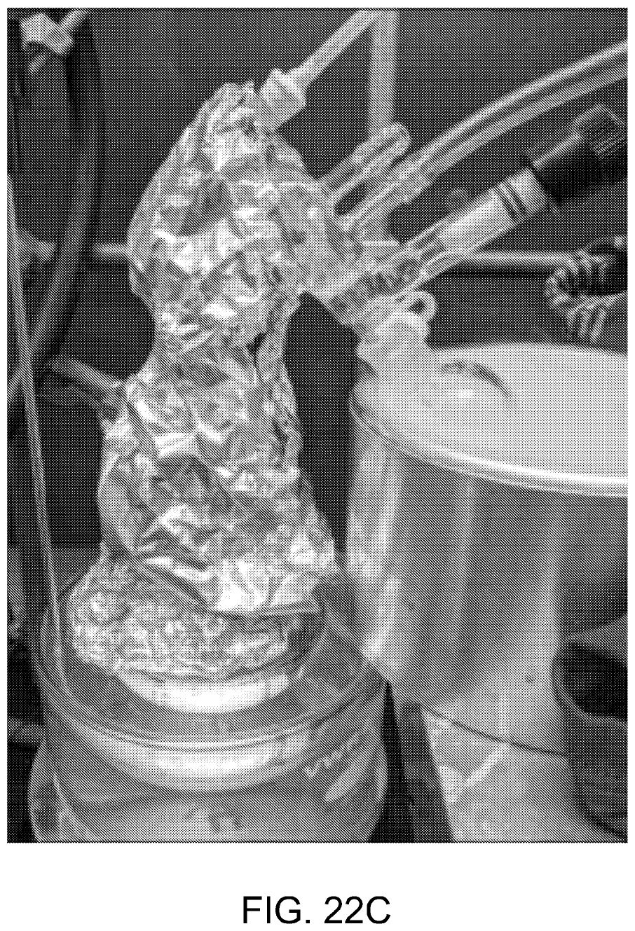

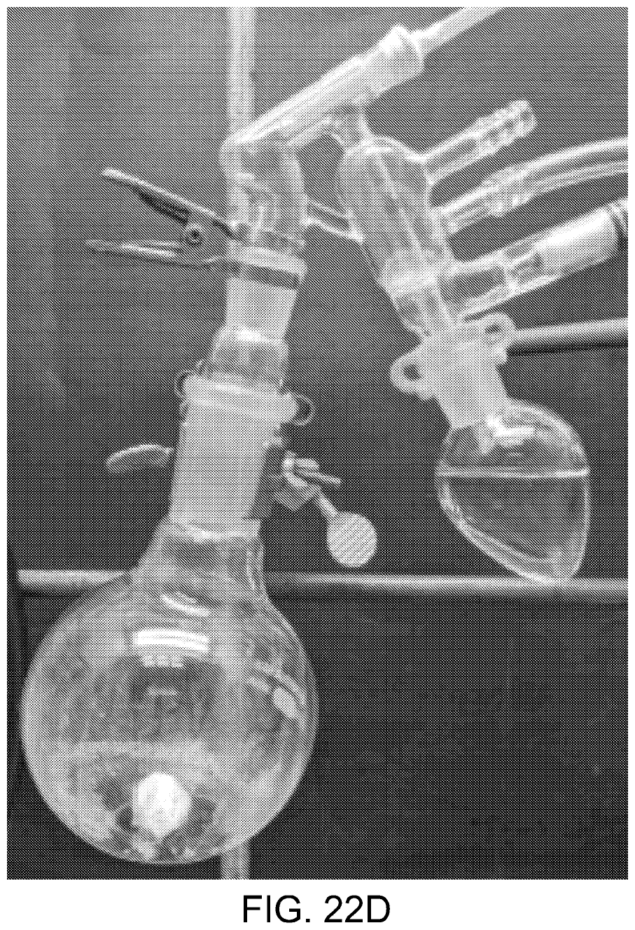

Monomer Recovery. PDXL depolymerization with camphorsulfonic acid—In a 100 mL round-bottom flask, PDXL (20.0 g) obtained from several batches of polymer (M_{n,GPC}=60-180 kDa, Ð=1.80-2.0) (FIG. 22A) was dissolved in CH₂Cl₂ (50 mL). Camphorsulfonic acid (CSA) (2 mol %, 1.26 g) was then added to the solution followed by stirring at room temperature for 30 minutes until fully dissolved. Then, solvent was removed via rotary evaporation. No heat was applied to the mixture prior to distillation. The homogenous polymer/acid mixture was fitted with a short-path distillation apparatus under ambient temperature and pressure (FIG. 22B). The receiving flask was cooled with a dry ice/isopropanol bath. The mixture was placed into a pre-heated 140° C. oil bath (FIG. 22C). First distillate collection was observed after 14 minutes. After 78 minutes, the distillation had finished and was removed from heat. Monomer was collected (19.5 g, 98% yield) and analyzed for purity by ¹H NMR (FIG. 22D). A small amount of CSA was observed in the ¹H NMR spectrum; residual acid in the monomer can be prevented by using a less volatile acid catalyst (e.g. reusable polymer-supported acid resin). In this experiment, residual CSA was removed by drying the monomer over CaH₂, followed by isolation of pure DXL by vacuum transfer.

TABLE 22
Monomer recovery from pristine feedstock
with 2 mol % CSA (1.26 g).

| Mₙ (kDa) | Time to 75% (min) |
|---|---|
| 39.9 | 6.7 |
| 111 | 9.0 |
| 220 | 10.5 |

Repolymerization.

TABLE 23
Stress, strain, and Young's Modulus values for PDXL (Mn =
84.4 kDa) taken from five replicates of tensile tests at a strain
rate of 5 mm/min for PDXL synthesized from recovered DXL feedstock.

| Sample Number | Stress (MPa)^{a} | Strain (%)^{b} | E′^{c} |
|---|---|---|---|
| 1 | 31.6 | 594 | 0.27 |
| 2 | 32.3 | 588 | 0.28 |
| 3 | 32.6 | 592 | 0.29 |
| 4 | 27.7 | 487 | 0.20 |
| 5 | 27.0 | 621 | 0.26 |

^{a}Stress at break
^{b}Strain at break.
^{c}Young's modulus calculated as the slope from 0 to 1% strain.

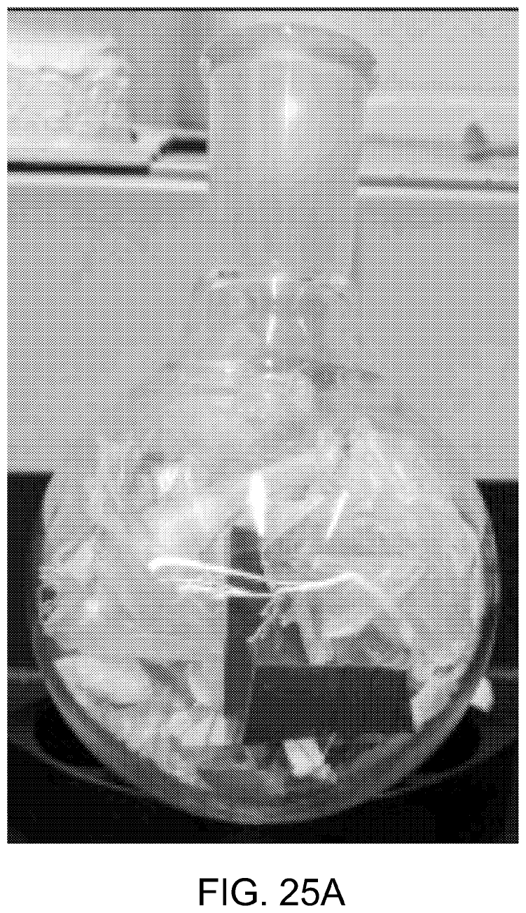

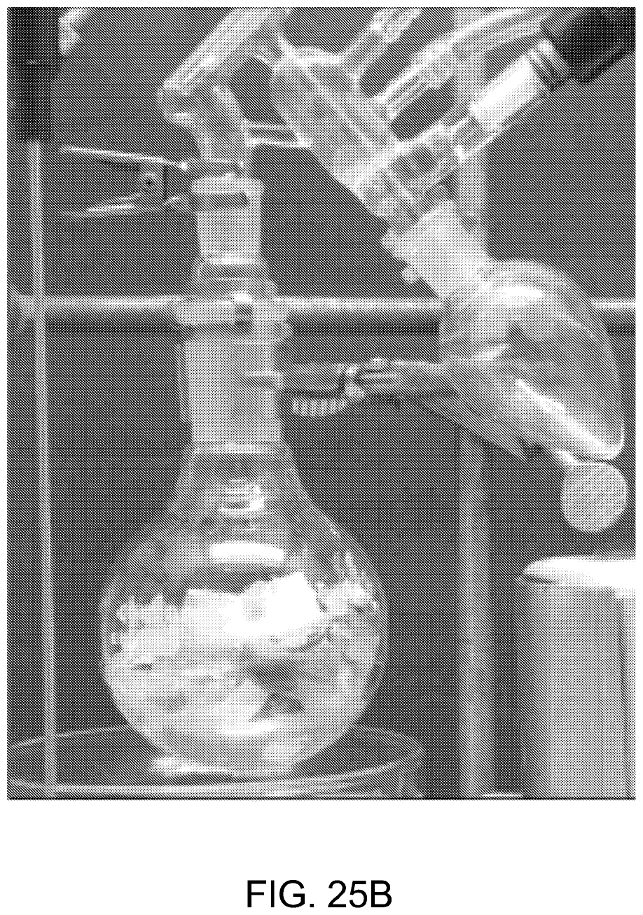

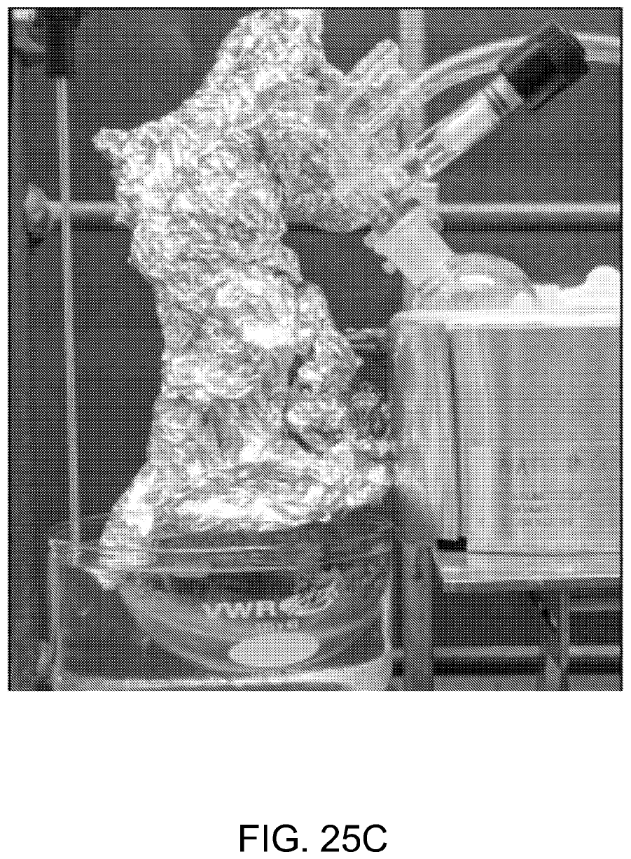

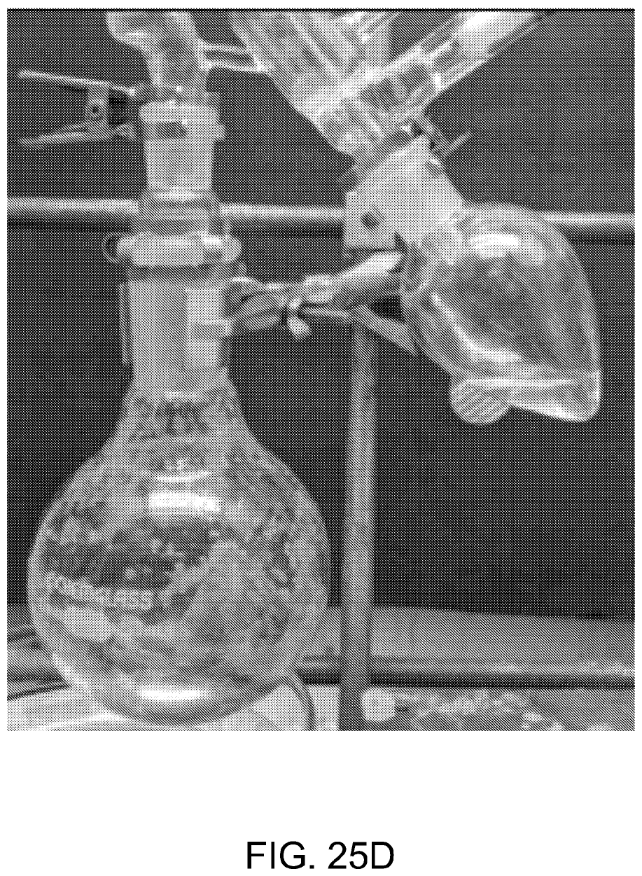

PDXL Depolymerization from Mixed Feedstock with Polymer-Supported Acid Resin—DOWEX-50 (3.45 g, 20 wt. %) was loaded into a 250 mL round-bottom flask. Then, small pieces of polystyrene (red disposable cup and clear plastic cup, 3.43 g), HDPE (obtained from a recycling facility, 5.00 g), LDPE (obtained from a recycling facility, 3.08 g), low molecular weight PEO (Sigma-Aldrich, viscosity modifier, 30 mL), and PDXL (3.22 g) (FIG. 25A, Table 24) were combined into a round bottomed flask (FIG. 25B). The flask was fitted with a short-path distillation head open to ambient temperature and pressure, and the receiving flask was cooled in a dry ice/isopropanol bath. The mixture was then placed in a pre-heated oil bath at 140° C. and stirred with a large magnetic stir bar for 2 h (FIG. 25C). A second portion of PDXL (2.09 g) was added after 1 h to demonstrate continuous depolymerization from a mixed polymer feed stock using a polymer-supported acid depolymerization catalyst. Clear, colorless distillate was collected in two fractions (F₁=3.14 g, F₂=2.01 g) (FIG. 25D). After 2 h, the distillation was placed under high vacuum with a new receiving flask that was cooled in a liquid N₂ bath to drive over any remaining distillate (F₃=0.805 g).

TABLE 24
Composition of polymer mixture used in depolymerization
study from mixed feedstocks.

| Component | Initial Mass (g) |
|---|---|
| PDXL (1) | 3.2 |
| PDXL (2) | 2.1 |
| HDPE | 5.0 |
| LDPE | 3.1 |
| PS | 3.4 |
| PEO | 30 mL |

To analyze the water content of the collected distillate, a ¹H NMR spectrum of anhydrous d₆-acetone from a sealed glass ampule was first taken to determine the water content in the solvent (integration=1.00:0.58 C₃H₆O:H₂O). Then, the same solvent was used to analyze each DXL distillate fraction (results calculated in Table 25), which contained 62, 48, and 93 mol % H₂O, respectively. The rest of the sample was pure DXL monomer (35, 52, and 7 mol %, respectively) to give an overall yield of 77.2 wt. % recovered DXL.

TABLE 25
Monomer recovery data from mixed feedstock with 20 wt. %
DOWEX-50 resin (3.45 g). PDXL was added in two portions
and distillation occurred at 140º C. for 2 h.
Distillate was collected in 3 fractions for 77% by mass total recovery.

| | | | | | | DXL |
|---|---|---|---|---|---|---|
| | Distillate | mol % | mol % | wt % | wt % | recovered |
| Fraction | (g) | H₂O | DXL | H₂O | DXL | (g) |
| F1 | 3.14 | 62 | 38 | 28 | 72 | 2.25 |
| F2 | 2.01 | 48 | 52 | 18 | 82 | 1.64 |
| F3 | 0.80 | 93 | 7 | 76 | 24 | 0.19 |

### EXAMPLE 2

The following is an example of chemically recyclable polyacetals with polyolefin-like properties, methods of making same, and methods of using same.

Ultra high molecular weight poly(cyclic acetal) is accessible with loadings as low as 0.0025 mol % organic cation salts, particularly oxonium salts and trityl salts, in the presence of the hindered base, di-tertbutyl pyridine. Di-tertbutyl pyridine functions as a proton trap to eliminate protic impurities. Without protic impurities in the polymerization system, the polymerization has higher chain end fidelity, allowing extremely low catalyst loadings and high conversions in cyclic acetal polymerization. Additionally, the proton trap allows the chain ends to avoid premature termination by water, avoiding protic chain ends and allowing chain end functionalization. Suitable organic cation catalysts are in the form of C⁺A⁻, where suitable C⁺ groups include electrophilic alkylating agents such as, for example, carbenium, carboxonium, trityl, oxycarbenium, or oxonium, particularly oxycarbenium or trityl, while suitable A⁻ groups include tetrafluoroborate (BF₄⁻), hexafluorophosphate (PF₆⁻), perchlorate (ClO₄⁻), triflate (SO₃CF₃⁻), tetrakis[3,5-bis(trifluoromethyl)phenyl]borate (BArF₂₄⁻), hexafluoroantimonate, hexachloroantimonate, tetrakis(pentafluorophenyl)borate B(C₆F₅)₄⁻, perfluoroalkyl aluminates, other non-nucleophilic anions, complex anions, or non-complex anions.

Synthesis of High MW Poly(1,3-dioxolane) (PDXL). In a glovebox under nitrogen atmosphere, 1,3-dioxolane (10.0 g, 135 mmol), n di-tert-butyl pyridine (0.017 mmol, 3.22 mg), and CH₂Cl₂ were combined in a 100 mL round bottom flask with a stir bar to give a final volume of 15 mL ([DXL]₀=9.0 M). In a separate vial, Et₃OPF₆ (83.7 mg, 0.34 mmol) was dissolved in anhydrous dichloromethane (10.0 mL), and sealed with a pierceable cap. The Et₃OPF₆ stock solution and polymerization flask were removed from the glovebox, and the reaction mixture was cooled to 0° C. in an ice bath for 3-5 min. The polymerization was initiated by addition of an Et₃OPF₆ stock solution (0.1 mL) directly to the reaction mixture under nitrogen using a gas-tight syringe. After the reaction became too viscous to stir, the reaction mixture was quenched by adding 5.0 mL of a 1.0 M solution of sodium benzyloxide in THF and mixing vigorously at 0° C. In some cases, mechanical stirring was required due to the high viscosity of the reaction mixture. Once quenched, the reaction mixture was warmed to room temperature and diluted with CH₂Cl₂ (30-150 mL) until reaching a viscosity suitable for precipitation. The reaction mixture was purified by first precipitating into cold diethyl ether (1 L) to give a white fibrous solid. The solid was redissolved in CH₂Cl₂ (30-150 mL) until reaching an appropriate viscosity for vacuum filtration. The solution was then stirred with K₂CO₃ (20.0 g) for 1 h to quench any residual acidic species. The K₂CO₃ was removed via vacuum filtration through a basic alumina and Celite pad, and the polymer was isolated by reprecipitating into cold diethyl ether (1.0 L). The white fibrous solid was isolated via vacuum filtration, dried at room temperature under vacuum overnight then dried under vacuum at 80° C. for 24 h to remove residual solvent and give a semi-crystalline white solid product. Similar reactions were performed with various ratios of DXL to Et₃OPF₆ (shown in Table 26). Aliquots were taken from each quenched reaction for NMR (CDCl₃) and GPC (THF) analysis. Theoretical molecular weights (M_{n,th}) were calculated as described herein. Spectral data match previous reports. ¹H NMR (500 MHz, CDCl₃): δ4.76 (s, 2H), 3.72 (s, 4H) ppm. 13C NMR (125 MHz, CDCl₃): δ95.7, 67.0 ppm. T_{g}=−62° C., Tₘ=58° C. δ=1.21 g/mL.

TABLE 26

| | | | Conv | Mₙₑₓₚ | Mₙₜₕₑₒ | |
|---|---|---|---|---|---|---|
| [DXL]:[Et₃OPF₆] | time | | (%) | (kDa) | (kDa) | Ð |
| 1000:1 | 40 | min | 87 | 129 | 74 | 1.75 |
| 2000:1 | 40 | min | 84 | 132 | 148 | 1.65 |
| 4000:1 | 70 | min | 85 | 233 | 312 | 1.59 |
| 8000:1 | 240 | min | 77 | ^{a} | — | — |
| 16,000:1 | 300 | min | 75 | ^{a} | — | — |
| 20,000:1 | 24 | h | ~80 | ^{a} | — | — |
| 40,000:1 | 24 | h | ~80 | ^{a} | — | — |

^{a} Exceeded GPC detection limit.

Diethoxymethane (DEM) as a chain transfer agent (CTA). Molecular weight of the polymer can be controlled by varying [DXL]:[DEM] stoichiometry. Using a CTA along with the di-tertbutyl pyridine proton trap allows only a very small amount of catalyst to be necessary to achieve poly(cyclic acetal)s of 10 kDa-3,000 kDa. Reactions were performed according to Scheme 7 using various equivalents of DEM (Table 27). Aliquots were taken from each quenched reaction for GPC (THF) analysis.

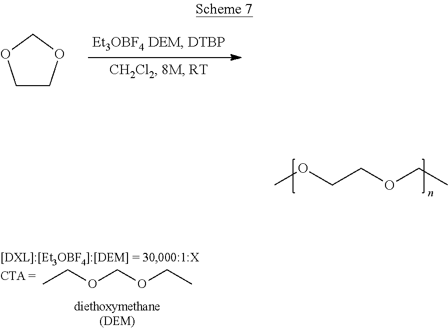

TABLE 27

| Equiv DEM | time | Conv (%) | Mₙₑₓₚ (kDa) | Ð |
|---|---|---|---|---|
| 10 | 24 h | 83 | 219 | 1.63 |
| 20 | 24 h | 87 | 144 | 1.68 |
| 40 | 24 h | 85 | 69.4 | 1.71 |
| 60 | 24 h | 82 | 48.8 | 1.68 |
| 80 | 24 h | 79 | 34.4 | 1.69 |
| 100 | 24 h | 74 | 28.8 | 1.66 |

Although the present disclosure has been described with respect to one or more particular example(s), it will be understood that other examples of the present disclosure may be made without departing from the scope of the present disclosure.

1. A poly(cyclic acetal) comprising: a number-average molecular weight (Mₙ) of from about 10 kiloDaltons (kDa) to about 3000 kDa, and wherein greater than about 50% of the poly(cyclic acetal) chain ends are not hydroxyl groups.

2. The poly(cyclic acetal) of claim 1, wherein the number-average molecular weight (Mₙ) is from about 100 kiloDaltons (kDa) to about 1000 kDa.

3. The poly(cyclic acetal) of claim 1, wherein the chain ends are independently chosen at each occurrence from alkyl ether groups, aryl ether groups, alkyl aryl ether groups, heterocyclic groups, halide groups, triflate groups, amine groups, thiol groups, or phosphine groups.

4. The poly(cyclic acetal) of claim 1, wherein the poly(cyclic acetal) comprises one or more homopolymer(s) or copolymer(s) of poly(1,3-dioxepane) (PDXP), poly(1,3-dioxocane) (PDXC), poly (1,3,6-trioxocane) (PTXC), poly(1,3-hexahydrobenzodioxole) (PHBD), poly(1,3-dioxolane) (PDXL), or any combination thereof.

5. The poly(cyclic acetal) of claim 1, wherein the poly(cyclic acetal) comprises one or more or all of the following:
a thermal stability (T_{d,5%}) of from about 337° C. to about 392° C.;
a thermal stability (T_{d,50%}) of from about 377° C. to about 462° C., with or without 2 mol % of one or more additive(s) having a pKₐ greater than 4;
an Arrhenius activation energy (Eₐ) of about 85.0 kJ/mol, with 2 mol % of one or more acid(s) having a pKₐ less than or equal to 4;
a melting temperature (Tₘ) of from about 0° C. to about 120° C.;
a glass transition temperature (T_{g}) of from about −100° C. to about 100° C.;
a tensile stress at break about 10 MPa to about 50 MPa; or
a tensile strain at break of from about 1% to about 800.

6. The polymer of claim 1, wherein the poly(cyclic acetal) is in the form of a solution, an emulsion, a slurry, a dispersion, a particle, a flake, a pellet, a powder, a granule, a tube, a sphere, a fiber, a foam, a film, a textile, a mesh, a sheet, a bar, or a monolith.

7. An article of manufacture comprising one or more poly(cyclic acetal(s)) of claim 1.

8. The article of manufacture of claim 7, wherein the article is a spun article, a molded article, an extruded article, a coated article, a blown article, a woven article, a drawn article, a laminated article, or a 3D printed article.

9. A method of making a poly(cyclic acetal), the method comprising:
combining:
one or more cyclic acetal monomer(s);
one or more haloalkyl ether initiator(s); and
one or more Lewis acid catalyst(s),
to form a polymerization mixture, wherein a poly(cyclic acetal) is formed.

10. The method of claim 9, wherein the cyclic acetal monomer(s) is/are cyclic methylene acetal monomer(s) chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and any combination thereof.

11. The method of claim 9, wherein the cyclic acetal monomer(s) is/are present in the polymerization mixture at from about 1.5 M to about 15 M or neat.

12. The method of claim 9, wherein the haloalkyl ether initiator(s) comprise(s) a C₁-C₂₅ haloalkyl group chosen from linear, cyclic, branched, substituted alkyl, unsaturated, and saturated C₁-C₂₅ haloalkyl group(s), and any combination thereof.

13. The method of claim 9, wherein the haloalkyl ether initiator(s) is/are chosen from benzyl chloromethyl ether, bromomethyl methyl ether, 1-chloromethyl adamantane, chloromethyl cyclohexyl ether, chloromethyl dococosyl ether, chloromethyl ethyl ether, chloromethyl methyl ether, tert-butyl chloromethyl ether, methoxyethyl chloromethyl ether, and any combination thereof.

14. The method of claim 9, wherein: the haloalkyl ether initiator(s) is/are present in the polymerization mixture at 1×10⁻⁶ mM to 1 mM; the initial molar ratio of the cyclic acetal monomer(s) to the haloalkyl ether initiator(s) is from about 200:1 to about 1600:1; or both.

15. The method of claim 9, wherein the Lewis acid catalyst(s) is/are Lewis acid(s) of the form MXₙ, wherein M=Ga, In, Zn, Sb^{V}, Sn^{IV}, or Fe, and wherein X=Cl, Br, I or OTf.

16. The method of claim 9, wherein: the Lewis acid catalyst(s) is/are present in the polymerization mixture at 1×10⁻⁵ mM to 2 mM; the initial molar ratio of the cyclic acetal monomer(s) to the Lewis acid catalyst(s) is from about 200:1 to about 1600:1; or both.

17. The method of claim 9, wherein the polymerization mixture further comprises one or more proton trap(s).

18. The method of claim 17, wherein the proton trap(s) is/are sterically hindered base(s) chosen from 2,6-di-tert-butylpyridine (DTPB), 2,6-di-tert-butyl-4-methylpyridine, 2,4,6-tri-tert-butylpyridine, 2,4,6-tri-tert-butylpyrimidine, and any combination thereof.

19. The method of claim 17, wherein: the proton trap(s) is/are present in the polymerization mixture at from about 2×10⁻⁵ mM to about 1 mM; the initial molar ratio of the cyclic acetal monomer(s) to the proton trap(s) is from about 200:1 to about 1600:1; or both.

20. The method of claim 9, wherein the polymerization is carried out at a temperature of from about −90° C. to about 50° C.

21. The method of claim 9, wherein the monomer to polymer conversion is from about 20% to about 100% conversion.

22. The method of claim 9, the method further comprising, after forming the poly(cyclic acetal), one or more or all of the following:
adding one or more quenching agent(s) to the polymerization mixture;
adding one or more base(s) to the polymerization mixture;
removing residual acidic species in the polymerization mixture; or
removing or isolating the poly(cyclic acetal) from the polymerization mixture.

23. A polymerization method for generating a poly(cyclic acetal), the method comprising:
combining:
one or more cyclic acetal monomer(s);
one or more organic cation salt catalyst(s); and
one or more proton trap(s),
to form a polymerization mixture, wherein a poly(cyclic acetal) is formed.

24. The method of claim 23, wherein the cyclic acetal monomer(s) is/are cyclic methylene acetal monomer(s) chosen from 1,3-dioxolane (DXL), 1,3 dioxepane (DXP), 1,3-dioxecane (DXC), 1,3,6-trioxane (TXC), trans-hexahydro-1,3-benzodioxole (HBD), and any combination thereof.

25. The method of claim 23, wherein the cyclic acetal monomer(s) is/are present in the polymerization mixture at from about 1.5 M to about 15M or neat.

26. The method of claim 23, wherein the organic cation salt catalyst(s) is/are of the form C⁺A⁻, wherein C⁺ is chosen from electrophilic alkylating agents, and wherein A⁻ is chosen from non-nucleophilic anions, complex anions, non-complex anions, and any combination thereof.

27. The method of claim 26, wherein C⁺=carbenium, carboxonium, trityl, oxycarbenium, or oxonium and A⁻=tetrafluoroborate (BF₄⁻), hexafluorophosphate (PF₆⁻), triflate (SOhd3CF₃⁻), perchlorate (ClO₄⁻), hexafluoroantimonate, hexachloroantimonate, perfluoroalkyl aluminates, tetrakis[3,5-bis(trifluoromethyl)phenyl]borate (BArF₂₄⁻), or tetrakis(pentafluorophenyl)borate (B(C₆F₅)₄⁻).

28. The method of claim 27, wherein the organic cation salt catalyst(s) is/are chosen from [(Et)₃O]BF₄, [(Et)₃O]PF₆, and any combination thereof.

29. The method of claim 23, wherein: the organic cation salt catalyst(s) is/are present in the polymerization mixture at 1×10⁻⁵ mM to 10 mM; the initial molar ratio of the cyclic acetal monomer(s) to the organic cation salt catalyst(s) is from about 1000:1 to about 40,000:1; or both.

30. The method of claim 23, wherein the proton trap(s) is/are sterically hindered base(s) chosen from 2,6-di-tert-butylpyridine, 2,6-di-tert-butyl-4-methylpyridine, 2,4,6-tri-tert-butylpyridine, 2,4,6-tri-tert-butylpyrimidine, and any combination thereof.

31. The method of claim 30, wherein: the proton trap(s) is/are present in the polymerization mixture at from about 1×10⁻⁵ mM to about 10 mM; the initial molar ratio of the cyclic acetal monomer(s) to the proton trap(s) is from about 200:1 to about 40,000:1; or both.

32. The method of claim 23, wherein the polymerization mixture further comprises one or more chain transfer agent(s) (CTA(s)).

33. The method of claim 32, wherein the chain transfer agent (CTA(s)) is/are acyclic acetal(s).

34. The method of claim 32, wherein: the chain transfer agent(s) (CTA(s)) is/are present in the polymerization mixture at from about 1×10⁻⁴ mM to about 6 mM; the initial molar ratio of the organic cation catalyst(s) to the CTA(s) is from about 1:1 to about 200:1; or both.

35. The method of claim 23, wherein the polymerization is carried out at a temperature of from about −90° C. to about 50° C.

36. The method of claim 23, wherein the monomer to polymer conversion is from about 20% to about 100% conversion.

37. The method of claim 23, the method further comprising, after forming the poly(cyclic acetal), one or more or all of:
adding one or more quenching agent(s) to the polymerization mixture;
adding one or more base(s) to the polymerization mixture;
removing residual acidic species in the polymerization mixture; or
removing or isolating the poly(cyclic acetal) from the polymerization mixture.

38. A depolymerization method comprising:
combining:
one or more poly(cyclic acetal(s)); and
one or more acid catalyst(s) having a pKₐ less than or equal to 4, to form a depolymerization mixture; and
heating the depolymerization mixture at a pressure of from about 1×10⁻⁴ atm to about 5.0 atm, to form said cyclic acetal(s).

39. The depolymerization method of claim 38, wherein the acid catalyst(s) is/are chosen from camphorsulfonic acid (CSA), diphenylphosphoric acid (DPA), sulfonic acid(s) (SA(S)), phosphoric acid(s) (PA(s)) and any combination thereof.

40. The depolymerization method of claim 38, wherein the acid catalyst(s) comprise(s) a solid support.

41. The depolymerization method of claim 38, wherein the depolymerization mixture comprises greater than or equal to 0.01 mol % of acid or greater, based on the total moles of poly(cyclic acetal) and acid catalyst(s).

42. The depolymerization method of claim 38, wherein the depolymerization mixture is heated to a temperature of from about 25° C. to about 300° C.

43. The depolymerization method of claim 38, further comprising removing or isolating said cyclic acetal(s) from the depolymerization mixture.
